SEQUENCE LISTING (11 sequences)

SEQ ID NO: 1 - DNA, 21 bp, Artificial Sequence
  synthetic nucleotide - siRNA s11836 (sense)
gcgaguuuguugugacauutt

SEQ ID NO: 2 - DNA, 21 bp, Artificial Sequence
  synthetic nucleotide - siRNA s21145 (sense)
ggcaguaaaggaaaucgaatt

SEQ ID NO: 3 - DNA, 21 bp, Artificial Sequence
  synthetic nucleotide - siRNA s15748 (sense)
cagacgagagugaaacugatt

SEQ ID NO: 4 - DNA, 21 bp, Artificial Sequence
  synthetic nucleotide - siRNA s15775 (sense)
caaccaugcaacuucgaaatt

SEQ ID NO: 5 - DNA, 21 bp, Artificial Sequence
  synthetic nucleotide - siRNA s11836 (antisense)
aaugucacaacaaacucgcca

SEQ ID NO: 6 - DNA, 21 bp, Artificial Sequence
  synthetic nucleotide - siRNA s21145 (antisense)
uucgauuuccuuuacugccgt

SEQ ID NO: 7 - DNA, 21 bp, Artificial Sequence
  synthetic nucleotide - siRNA s15748 (antisense)
ucaguuucacucucgucuggg

SEQ ID NO: 8 - DNA, 21 bp, Artificial Sequence
  synthetic nucloetide - siRNA s15775 (antisense)
uuucgaaguugcaugguugtc

SEQ ID NO: 9 - DNA, 21 bp, Artificial Sequence
  synthetic primer sequence - forward
ccgtctgtgccttctcatctg

SEQ ID NO: 10 - DNA, 29 bp, Artificial Sequence
  synthetic primer sequence - reverse
agtccaagagtyctcttatgyaagacctt

SEQ ID NO: 11 - DNA, 26 bp, Artificial Sequence
  synthetic oligonucleotide dual-labeled probe
  misc_feature (1)..(1): 5′ end labeled with fluorescein (FAM)
  misc_feature (26)..(26): 3′ end labeled with black hole quencher (BHQ1)
ccgtgtgcacttcgcttcacctctgc

(19) United States
(12) Patent Application Publication
(10) Pub. No.: US 2018/0042905 A1
(43) Pub. Date: Feb. 15, 2018

(54) METHODS OF TREATING HEPATITIS B VIRUS

(71) Applicant: Gilead Sciences, Inc., Foster City, CA (US)

(72) Inventors: Esmeralda Aguayo, Modesto, CA (US); Todd Appleby, San Francisco, CA (US); Gabriel Birkus, San Francisco, CA (US); Guofeng Cheng, Foster City, CA (US); David Dornan, Burlingame, CA (US); Tetsuya Kobayashi, Pleasanton, CA (US); Christopher Charles Mello, Sunnyvale, CA (US); Uli Schmitz, Foster City, CA (US); Madeleine Willkom, Foster City, CA (US); Mei Yu, Foster City, CA (US)

(21) Appl. No.: 15/689,985

(22) Filed: Aug. 29, 2017

Related U.S. Application Data
(63) Continuation of application No. 15/098,112, filed on Apr. 13, 2016, now abandoned.
(60) Provisional application No. 62/147,400, filed on Apr. 14, 2015.

(51) Int. Cl. A61K 31/44; A61K 45/06

(52) U.S. Cl. CPC A61K 31/44; A61K 45/06

(57) ABSTRACT

The present invention relates to novel methods of treating Hepatitis B Virus by administering a KDM5 inhibitor.

### CROSS-REFERENCE TO RELATED APPLICATIONS

This application claims the benefit under 35 U.S.C. §119(e) of U.S. Provisional Application Ser. No. 62/147,400, filed Apr. 14, 2015, the entirety of which is incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 1113PC.txt, date recorded: Apr. 12, 2016, size: 2.96 KB).

### FIELD

The present invention relates to novel methods of treating Hepatitis B Virus by administering a KDM5 inhibitor.

### BACKGROUND

Hepatitis B Virus (HBV) is an enveloped DNA virus belonging to the Hepadnaviridae family. HBV is classified into ten genotypes, A through J, which influence varying degrees of disease severity, risk of developing hepatocellular carcinoma (HCC), and response to interferon-α (IFN-α therapies. In the host cell's nucleus, the HBV's partially double-stranded relaxed circular DNA (rcDNA) genome is converted into covalently closed circular DNA (cccDNA) which persists as a nucleosome-bound minichromosome. The latter provides templates for future viral RNA transcription yielding new pregenomic viral RNA and the mRNAs for the HBV proteins, including the secreted HBV s- and e-antigens. (Zeisel M B, et al. Gut 2015; 0:1-13. doi:10.1136/gutjnl-2014-308943).

Current nucleoside-based HBV therapies prevent the reverse transcription of pregenomic H-BV RNA into fully functional IBV rcDNA such that new cccDNA is no longer formed. Theoretically, a single copy of cccDNA could reactivate a full infection. (Zeisel M B, et al. Gut 2015; 0:1-13. doi: 10.1136/gutjnl-2014-308943). However, current nucleoside antivirals have no effect on the existing HBV cccDNA from the pre-treatment period. Little is known about the persistence and transcriptional activity of HBV cccDNA, but it is likely that it is being regulated by host epigenetic factors.

More than 240 million individuals worldwide are chronically infected with Hepatitis B Virus (HBV). Treatments for infected individuals comprise IFN-α, pegylated (PEG)-IFN-α, and nucleoside analogues, however low sustained virological response (SVR) rates and adverse effects leave most patients on long-term treatments. For the majority of these individuals, there is no cure. Only some achieve HBV surface antigen (HBsAg) seroconversion, which is when the number of HBsAg-specific antibodies exceeds the number of HBsAg. (Zeisel M B, et al. Gut 2015; 0:1-13. doi:10.1136/gutjnl-2014-308943).

Thus, there is a need for compositions and methods of treating HBV infections. The present invention addresses these and other needs.

### SUMMARY

The present invention provides novel methods for treating HBV. A specific embodiment of the invention provides a method of treating HBV comprising administering a KDM5 inhibitor to a patient infected with HBV. In a further embodiment, the method of treating HBV comprises administering a KDM5 inhibitor to the patient once daily. In a further embodiment the method of treating HBV comprises administering a KDM5 inhibitor in a pulse dosing regimen.

In some embodiments of the invention, the KDM5 inhibitor inhibits at least 2 isoforms of KDM5, selected from the group consisting of KDM5a, KDM5b, KDM5c, and KDM5d. In further embodiments of the invention, the KDM5 inhibitor inhibits at least 3 isoforms of KDM5, selected from the group consisting of KDM5a, KDM5b, KDM5c, and KDM5d. In another embodiment of the invention, the KDM5 inhibitor inhibits 4 isoforms of KDM5, selected from the group consisting of KDM5a, KDM5b, KDM5c, and KDM5d.

In some embodiments of the invention, the method of treating HBV further comprises administering an additional therapeutic agent. In some embodiments, the additional therapeutic agent is administered separately from the KDM5 inhibitor. In other embodiments, the additional therapeutic agent is administered in combination with the KDM5 inhibitor. A non-exhaustive list of additional agents includes adefovir, tenofovir disoproxil fumarate, tenofovir alafenamide hemifumarate, entecavir, interferon, lamivudine and telbivudine.

In some embodiments of the invention the method of treating HBV comprises administering a KDM5 inhibitor and tenofovir disoproxil. In some embodiments the tenofovir disoproxil may be tenofovir disoproxil fumarate, tenofovir disoproxil phosphate or tenofovir disoproxil succinate. Typically, the tenofovir disoproxil is tenofovir disoproxil fumarate. In some embodiments the KDM5 inhibitor and tenofovir disoproxil are administered separately. In other embodiments, the KDM5 inhibitor and tenofovir disoproxil are administered in combination. In any of these embodiments the KDM5 inhibitor may be a compound having the following structure:

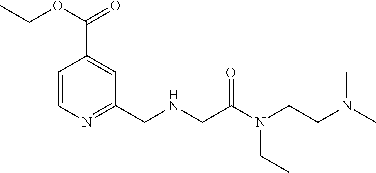 [SMILES: CCOC(=O)c1ccnc(CNCC(=O)N(CC)CCN(C)C)c1]

or a pharmaceutically acceptable salt thereof.

In some embodiments of the invention the method of treating HBV comprises administering a KDM5 inhibitor and tenofovir alafenamide. In some embodiments the tenofovir alafenamide may be tenofovir alafenamide monofumarate or tenofovir alafenamide hemifumarate. Typically, the tenofovir alafenamide is tenofovir alafenamide hemifumarate. In some embodiments the KDM5 inhibitor and tenofovir alafenamide are administered separately. In other embodiments, the KDM5 inhibitor and tenofovir alafenamide are administered in combination. In any of these embodiments the KDM5 inhibitor may be a compound having the following structure:

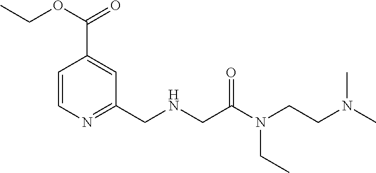 [SMILES: CCOC(=O)c1ccnc(CNCC(=O)N(CC)CCN(C)C)c1]

or a pharmaceutically acceptable salt thereof. In some embodiments of the invention the method of treating HBV comprises administering a KDM5 inhibitor and a TLR8 inhibitor. In some embodiments the KDM5 inhibitor and TLR8 inhibitor are administered separately. In other embodiments, the KDM5 inhibitor and TLR8 inhibitor are administered in combination. In any of these embodiments the KDM5 inhibitor may be a compound having the following structure:

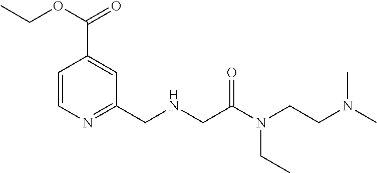 [SMILES: CCOC(=O)c1ccnc(CNCC(=O)N(CC)CCN(C)C)c1]

or a pharmaceutically acceptable salt thereof.

In some embodiments of the invention, the KDM5 inhibitor is siRNA. In some embodiments, the siRNA comprises a nucleic acid having SEQ ID NO. 1 or a nucleic acid having at least 90% identity to SEQ ID NO. 1.

In some embodiments of the invention, the KDM5 inhibitor is siRNA. In some embodiments, the siRNA comprises a nucleic acid having SEQ ID NO. 2 or a nucleic acid having at least 90% identity to SEQ ID NO. 2.

In some embodiments of the invention, the KDM5 inhibitor is siRNA. In some embodiments, the siRNA comprises a nucleic acid having SEQ ID NO. 3 or a nucleic acid having at least 90% identity to SEQ ID NO. 3.

In some embodiments of the invention, the KDM5 inhibitor is siRNA. In some embodiments, the siRNA comprises a nucleic acid having SEQ ID NO. 4 or a nucleic acid having at least 90% identity to SEQ ID NO. 4.

In some embodiments of the invention, the KDM5 inhibitor is siRNA In some embodiments, the siRNA comprises a nucleic acid having SEQ ID NO. 4 or a nucleic acid having at least 90% identity to SEQ ID NO. 5.

In some embodiments of the invention, the KDM5 inhibitor is siRNA. In some embodiments, the siRNA comprises a nucleic acid having SEQ ID NO. 6 or a nucleic acid having at least 90% identity to SEQ ID NO. 6.

In some embodiments of the invention, the KDM5 inhibitor is siRNA. In some embodiments, the siRNA comprises a nucleic acid having SEQ ID NO. 7 or a nucleic acid having at least 90% identity to SEQ ID NO. 7.

In some embodiments of the invention, the KDM5 inhibitor is siRNA. In some embodiments, the siRNA comprises a nucleic acid having SEQ ID NO. 8 or a nucleic acid having at least 90% identity to SEQ ID NO. 8.

In other embodiments, the KDM5 inhibitor is a compound of Formula I^{a}:

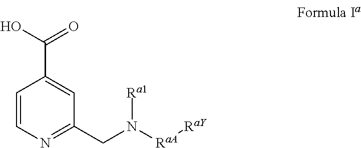

or a pharmaceutically acceptable salt thereof.

In other embodiments, the KDM5 inhibitor is a compound of Formula I^{a2}:

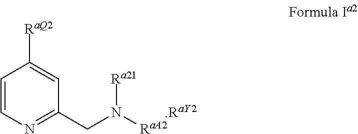

or a pharmaceutically acceptable salt thereof.

In other embodiments, the KDM5 inhibitor is a compound of Formula I^{b}:

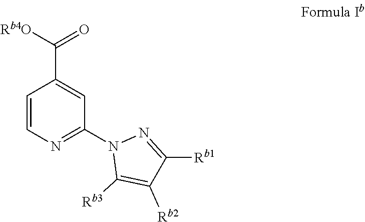

or a pharmaceutically acceptable salt thereof.

In other embodiments, the KDM5 inhibitor is a compound of Formula I^{b2}:

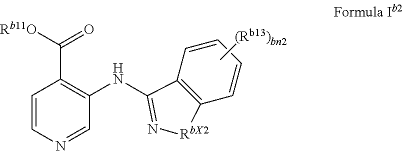

or a pharmaceutically acceptable salt thereof.

In other embodiments, the KDM5 inhibitor is a compound of Formula II^{b2}:

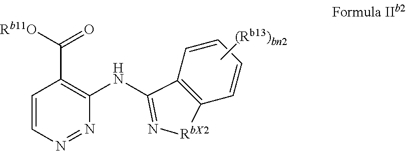

or a pharmaceutically acceptable salt thereof.

In other embodiments, the KDM5 inhibitor is a compound of Formula I^{b3}:

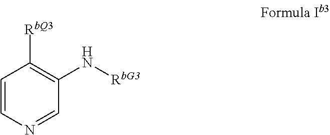

or a pharmaceutically acceptable salt thereof.

In a further embodiment, Formula I^{b3} has the structure of Formula I^{b3a}:

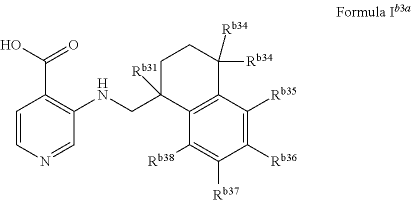

or a pharmaceutically acceptable salt thereof.

In a further embodiment, Formula I^{b3} has the structure of Formula I^{b3b}:

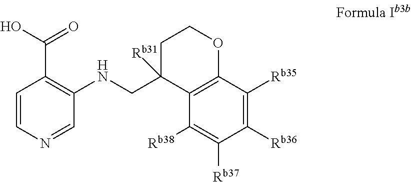

or a pharmaceutically acceptable salt thereof.

In other embodiments, the KDM5 inhibitor is a compound of Formula II^{b3}:

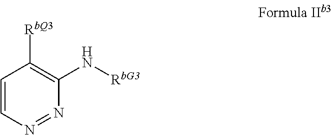

or a pharmaceutically acceptable salt thereof.

In other embodiments, the KDM5 inhibitor is a compound of Formula I^{b4}:

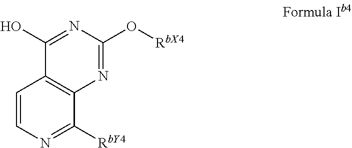

or a pharmaceutically acceptable salt thereof.

In other embodiments, the KDM5 inhibitor is a compound of Formula I^{b5}:

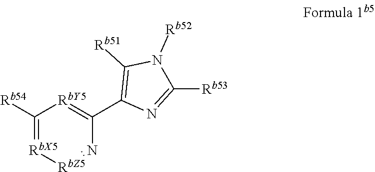

or a pharmaceutically acceptable salt thereof.

In other embodiments, the KDM5 inhibitor is a compound of Formula II^{b5}:

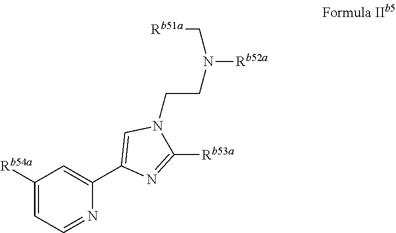

or a pharmaceutically acceptable salt thereof.

In other embodiments, the KDM5 inhibitor is a compound of Formula I^{b6}:

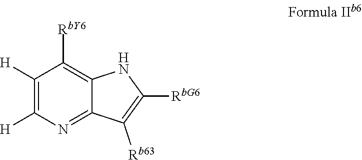

or a pharmaceutically acceptable salt thereof.

In a further embodiment, Formula I^{b6} has the structure of Formula II⁶:

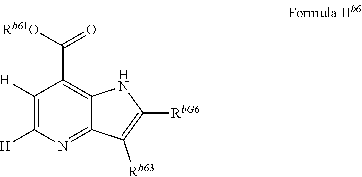

or a pharmaceutically acceptable salt thereof.

In other embodiments, the KDM5 inhibitor is a compound of Formula I^{c}:

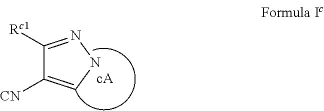

or a pharmaceutically acceptable salt thereof.

In some embodiments of the invention, the KDM5 inhibitor is a compound of Formula I^{c2}:

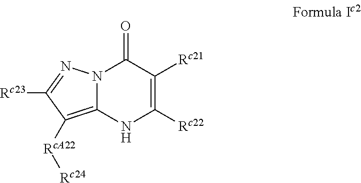

or a pharmaceutically acceptable salt thereof.

### DETAILED DESCRIPTION

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

A dash at the front or end of a chemical group is a matter of convenience; chemical groups may be depicted with or without one or more dashes without losing their ordinary meaning.

A dashed line indicates an optional bond. Where multiple substituent groups are identified the point of attachment is at the terminal substituent (e.g. for “alkylaminocarbonyl” the point of attachment is at the carbonyl substituent).

The prefix “C_{x-y}” indicates that the following group has from x (e.g. 1) to y (e.g. 6) carbon atoms, one or more of which, in certain groups (e.g. heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms. Likewise, the term “x-y membered” rings, wherein x and y are numerical ranges, such as “3-12 membered heterocyclyl”, refers to a ring containing x-y atoms (e.g. 3-12), of which up to half may be heteroatoms, such as N, O, S, P, and the remaining atoms are carbon.

Also, certain commonly used alternative chemical names may or may not be used. For example, a divalent group such as a divalent “alkyl” group, a divalent “aryl” group, etc., may also be referred to as an “alkylene” group or an “alkylenyl” group, or alkylyl group, an “arylene” group or an “arylenyl” group, or arylyl group, respectively.

### Definitions

The term “aliphatic” or “aliphatic group” refers to a hydrocarbon moiety that may be a straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)N(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

The term “alkoxy” as used herein refers to an “alkyl-O” group, wherein alkyl is as defined herein.

“Alkyl” refers to a straight or branched hydrocarbon chain radical consisting solely of carbon and hydrogen atoms, containing no unsaturation, having from one to fifteen carbon atoms (e.g., C₁₋₁₅ alkyl). In certain embodiments, an alkyl comprises one to thirteen carbon atoms (e.g., C₁₋₁₃ alkyl). In certain embodiments, an alkyl comprises one to eight carbon atoms (e.g., C₁₋₈ alkyl). In other embodiments, an alkyl comprises one to five carbon atoms (e.g., C₁₋₅ alkyl). In other embodiments, an alkyl comprises one to four carbon atoms (e.g., C₁₋₄ alkyl). In other embodiments, an alkyl comprises one to three carbon atoms (e.g., C₁₋₃ alkyl). In other embodiments, an alkyl comprises one to two carbon atoms (e.g., C₁₋₂ alkyl). In other embodiments, an alkyl comprises one carbon atom (e.g., C₁ alkyl). In other embodiments, an alkyl comprises five to fifteen carbon atoms (e.g., C₅₋₁₅ alkyl). In other embodiments, an alkyl comprises five to eight carbon atoms (e.g., C₅₋₈ alkyl). In other embodiments, an alkyl comprises two to five carbon atoms (e.g., C₂₋₅ alkyl). In other embodiments, an alkyl comprises two to ten carbon atoms (e.g., C₂₋₁₀ alkyl). In other embodiments, an alkyl comprises three to five carbon atoms (e.g., C₃₋₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1, 1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)N(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. In certain embodiments, an alkylene comprises one to eight carbon atoms (e.g., C₁₋₈ alkylene). In other embodiments, an alkylene comprises one to five carbon atoms (e.g., C₁₋₅ alkylene). In other embodiments, an alkylene comprises one to four carbon atoms (e.g., C₁₋₄ alkylene). In other embodiments, an alkylene comprises one to three carbon atoms (e.g., C₁₋₃ alkylene). In other embodiments, an alkylene comprises one to two carbon atoms (e.g., C₁₋₂ alkylene). In other embodiments, an alkylene comprises one carbon atom (e.g., C₁ alkylene). In other embodiments, an alkylene comprises five to eight carbon atoms (e.g., C₅₋₈ alkylene). In other embodiments, an alkylene comprises two to five carbon atoms (e.g., C₂₋₅ alkylene). In other embodiments, an alkylene comprises three to five carbon atoms (e.g., C₃₋₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}. —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The term “alkynylene” refers to a biradical (-alkynyl-).

The term “amine” as used herein refers to primary (R—NH₂, R≠H), secondary (R²—NH, R²≠H) and tertiary (R³—N, R≠H) amines. A substituted amine is intended to mean an amine where at least one of the hydrogen atoms has been replaced by the substituent.

“Amino” refers to the —NH₂ radical.

“Aralkenyl” refers to a radical of the formula —R^{d}-aryl where R^{d} is an alkenylene chain as defined herein. The aryl part of the aralkenyl radical is optionally substituted as described herein for an aryl group. The alkenylene chain part of the aralkenyl radical is optionally substituted as defined herein for an alkenylene group.

“Aralkoxy” refers to a radical bonded through an oxygen atom of the formula —O—R^{c}-aryl where R^{c} is an alkylene chain as defined herein, for example, methylene, ethylene, and the like. The alkylene chain part of the aralkyl radical is optionally substituted as described herein for an alkylene chain. The aryl part of the aralkyl radical is optionally substituted as described herein for an aryl group.

“Aralkyl” refers to a radical of the formula —R^{c}-aryl where R^{c} is an alkylene chain as defined herein, for example, methylene, ethylene, and the like. The alkylene chain part of the aralkyl radical is optionally substituted as described herein for an alkylene chain. The aryl part of the aralkyl radical is optionally substituted as described herein for an aryl group.

“Aralkynyl” refers to a radical of the formula —R^{e}-aryl, where R^{e} is an alkynylene chain as defined herein. The aryl part of the aralkynyl radical is optionally substituted as described herein for an aryl group. The alkynylene chain part of the aralkynyl radical is optionally substituted as defined herein for an alkynylene chain.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alknyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkyl ene or alkenylene chain, and R^{c} is a straight or branched alkylene or alkenylene chain, and where each of the above substituents is unsubstituted unless otherwise indicated.

The term “arylene” refers to biradical (-aryl-).

As used herein a “direct bond” or “covalent bond” refers to a single, double or triple bond. In certain embodiments, a “direct bond” or “covalent bond” refers to a single bond.

The term “carbamoyl” as used herein refers to a “H₂N(C═O)—” group.

“Carbocyclyl” refers to a stable non-aromatic monocyclic or polycyclic hydrocarbon radical consisting solely of carbon and hydrogen atoms, which may include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl may be saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2). —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

The terms “cycloaliphatic”, “carbocycle”, “carbocyclyl”, “carbocyclo”, or “carbocyclic”, used alone or as part of a larger moiety, refer to a saturated or partially unsaturated cyclic aliphatic monocyclic or bicyclic ring systems, as described herein, having from 3 to 10 members, wherein the aliphatic ring system is optionally substituted as defined herein and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3-6 carbons. The terms “cycloaliphatic”, “carbocycle”, “carbocyclyl”, “carbocyclo”, or “carbocyclic” also include aliphatic rings that are fused to one or more aromatic or nonaromatic rings, such as decahydronaphthyl, tetrahydronaphthyl, decalin, or bicyclo[2.2.2]octane, where the radical or point of attachment is on an aliphatic ring.

“Carbocyclylalkoxy” refers to a radical bonded through an oxygen atom of the formula —O—R^{c}-carbocyclyl where R^{c} is an alkylene chain as defined herein. The alkylene chain and the carbocyclyl radical is optionally substituted as defined herein.

“Carbocyclylalkyl” refers to a radical of the formula —R^{c}-carbocyclyl where R^{c} is an alkylene chain as defined herein. The alkylene chain and the carbocyclyl radical is optionally substituted as defined herein.

“C-heterocyclyl” or “C-attached heterocyclyl” refers to a heterocyclyl radical as defined herein containing at least one heteroatom and where the point of attachment of the heterocyclyl radical to the rest of the molecule is through a carbon atom in the heterocyclyl radical. A C-heterocyclyl radical is optionally substituted as described herein for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“C-heteroaryl” refers to a heteroaryl radical as defined herein and where the point of attachment of the heteroaryl radical to the rest of the molecule is through a carbon atom in the heteroaryl radical. A C-heteroaryl radical is optionally substituted as described herein for heteroaryl radicals.

“Cyano” refers to the —CN radical.

The term “cycloalkyl” as used herein refers to a cyclic alkyl group, preferably containing from three to ten carbon atoms (C₃₋₁₀-cycloalkyl), such as from three to eight carbon atoms (C₃₋₈-cycloalkyl), preferably from three to six carbon atoms (C₃₋₈-cycloalkyl), including cyclopropyl, cyclobutyl, cyclopentyl, cyclohexyl, cycloheptyl and cyclooctyl. Furthermore, the term “cycloalkyl” as used herein may also include polycyclic groups such as for example bicyclo[2.2.2]octyl, bicyclo[2.2.1]heptanyl, decalinyl and adamantyl.

The term “cycloalkylene” refers to biradical (-cycloalkyl-).

Illustrative examples of esters of a carboxylic acid group (in particular the pyridine carboxylic acid) are C₁₋₆ alkyl esters, e.g. methyl esters, ethyl esters, 2-propyl esters, phenyl esters, 2-aminoethyl esters, etc., including (5-methyl-2-oxo-2H-1,3-dioxol-4-yl)methyl esters, 4-methoxyphenyl esters, 2-(ethoxycarbonyl)phenyl esters, {4-[(ethoxycarbonyl)(methyl)amino]phenyl}methyl esters, 2-(dimethylamino)ethyl esters, 3-(dimethylamino)propyl esters, [(ethoxycarbonyl)amino]phenylmethyl esters, 2,6-dimethoxyphenyl esters, 2,6-dimethylphenyl esters, 4-tert-butylphenyl esters, 4-oxopentan-2-yl esters, 4-(trifluoroacetamido)butan-2-yl esters, 4-(2,2,2-trifluoro-N-methylacetamido)butan-2-yl esters, 5-(trifluoroacetamido)pent-1-en-3-yl esters, 5-(2,2,2-trifluoro-N-methylacetamido)pent-1-en-3-yl esters, 1,3-bis(hexadecanoyloxy)propan-2-yl esters, 2,3-bis(hexadecanoyloxy)propyl esters, 4-oxo-4-(propan-2-yloxy)-1-(trifluoroacetamido)butan-2-yl esters, 1-oxo-1-(propan-2-yloxy)-5-(trifluoroacetamido)pentan-3-yl esters 2,2,2-trifluoethyl esters, 2,6-bis(propan-2-yloxy)phenyl esters, 2-fluoroethyl esters, 2,2-difluoroethyl esters, etc.

“Fluoroalkyl” refers to an alkyl radical, as defined herein, that is substituted by one or more fluoro radicals, as defined herein, for example, trifluoromethyl, difluoromethyl, fluoromethyl, 2,2,2-trifluoroethyl, 1-fluoromethyl-2-fluoroethyl, and the like. The alkyl part of the fluoroalkyl radical may be optionally substituted as defined herein for an alkyl group.

The term “geometric isomer” refers to E or Z geometric isomers {e.g., cis or trans) of an alkene double bond. The term “positional isomer” refers to structural isomers around a central ring, such as ortho-, meta-, and para-isomers around a benzene ring.

“Halo” or “halogen” refers to bromo, chloro, fluoro or iodo substituents.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Huckel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[¾][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5.6.7.8-tetrahydrobenzo[4,5]thieno[2,3-d]pyrimidinyl, 6.7.8.9-tetrahydro-5H-cyclohepta[4,5]thieno[2,3-d]pyrimidinyl, 5,6,7,8-tetrahdropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined herein which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R′)₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜOR^{a} (where t is 1 or 2) and —R^{b}—S(O)N(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroarylalkoxy” refers to a radical bonded through an oxygen atom of the formula —O—R^{c}-heteroaryl, where R^{c} is an alkylene chain as defined herein. If the heteroaryl is a nitrogen-containing heteroaryl, the heteroaryl is optionally attached to the alkyl radical at the nitrogen atom. The alkylene chain of the heteroarylalkoxy radical is optionally substituted as defined herein for an alkylene chain. The heteroaryl part of the heteroarylalkoxy radical is optionally substituted as defined herein for a heteroaryl group.

“Heteroarylalkyl” refers to a radical of the formula —R^{c}-heteroaryl, where R^{c} is an alkylene chain as defined herein. If the heteroaryl is a nitrogen-containing heteroaryl, the heteroaryl is optionally attached to the alkyl radical at the nitrogen atom. The alkylene chain of the heteroarylalkyl radical is optionally substituted as defined herein for an alkylene chain. The heteroaryl part of the heteroarylalkyl radical is optionally substituted as defined herein for a heteroaryl group.

The term “heteroarylene” refers to biradical (-heteroaryl-).

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined herein that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heterocyclylalkoxy” refers to a radical bonded through an oxygen atom of the formula —O—R^{c}-heterocyclyl where R^{c} is an alkylene chain as defined herein. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined herein for an alkylene chain. The heterocyclyl part of the heterocyclylalkoxy radical is optionally substituted as defined herein for a heterocyclyl group.

“Heterocyclylalkyl” refers to a radical of the formula —R^{c}-heterocyclyl where R^{c} is an alkylene chain as defined herein. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined herein for an alkylene chain. The heterocyclyl part of the heterocyclylalkyl radical is optionally substituted as defined herein for a heterocyclyl group.

Correspondingly, the term “heterocyclylene” means the corresponding biradical (-heterocyclyl-).

“Hydrazino” refers to the ═N—NH₂ radical.

The term “hydroxyalkyl” as used herein refers to an alkyl group (as defined herein), which alkyl group is substituted one or more times with hydroxy. Examples of hydroxyalkyl groups include HO—CH₂—, HO—CH₂—CH₂— and CH₃—CH(OH)⁻.

“Imino” refers to the ═N—H radical.

### Isomers

The compounds of Formulae I^{a}, I^{a2}, I^{b}, I^{b2}, II^{b2}, I^{b3}, I^{b3a}, I^{b3b}, II^{b3b}, I^{b4}, I^{b5}, I^{b6}, II^{b6}, I^{c} and I^{c2} may exist as geometric isomers (i.e. cis-trans isomers), optical isomers or stereoisomers, such as diastereomers, as well as tautomers. Accordingly, it should be understood that the definition of compounds of Formulae I^{a}, I^{a2}, I^{b2}, II^{b2}, I^{b3}, I^{b3a}, I^{b3b}, II^{b3}, I^{b4}, I^{b5}, I^{b6}, II^{b6}, I^{c} and I^{c2} includes each and every individual isomers corresponding to the structural formula; Formulae I^{a}, I^{a2}, I^{b}, I^{b2}, II^{b2}, I^{b3}, I^{b3a}, I^{b3b}, I^{b4}, I^{b5}, I^{b6}, II^{b6}, I^{c} and I^{c2}, including cis-trans isomers, stereoisomers and tautomers, as well as racemic mixtures of these and pharmaceutically acceptable salts thereof. Hence, the definition of compounds of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} is also intended to encompass all R- and S-isomers of a chemical structure in any ratio, e.g. with enrichment (i.e. enantiomeric excess or diastereomeric excess) of one of the possible isomers and corresponding smaller ratios of other isomers. Diastereoisomers, i.e. non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} can likewise be obtained by utilizing optically active starting materials and/or by utilizing a chiral catalyst. These isomers may be in the form of a free acid, a free base, an ester or a salt. Examples of chiral separation techniques are given in Chiral Separation Techniques, A Practical Approach, 2nd ed. by G. Subramanian, Wiley-VCH, 2001.

The compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} may exist as geometric isomers (i.e. cis-trans isomers), optical isomers or stereoisomers, such as diastereomers, as well as tautomers. Accordingly, it should be understood that the definition of compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2}

includes each and every individual isomers corresponding to the structural formula; Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, I^{c} and I^{c2}, including cis-trans isomers, stereoisomers and tautomers, as well as racemic mixtures of these and pharmaceutically acceptable salts thereof. Hence, the definition of compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} are also intended to encompass all R- and S-isomers of a chemical structure in any ratio, e.g. with enrichment (i.e. enantiomeric excess or diastereomeric excess) of one of the possible isomers and corresponding smaller ratios of other isomers. Diastereoisomers, i.e. non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formulae I^{a}, I^{a1}, I^{a2}, I^{b}, I^{b2}, II^{b2}, I^{b3a}, I^{b3b}, II^{b3}, I^{b4}, I^{b5}, II^{b5}, I^{b6}, II^{b6}, I^{c} and I^{c2} can likewise be obtained by utilizing optically active starting materials and/or by utilizing a chiral catalyst. These isomers may be in the form of a free acid, a free base, an ester or a salt. Examples of chiral separation techniques are given in Chiral Separation Techniques, A Practical Approach, 2nd ed. by G. Subramanian, Wiley-VCH, 2001.

“N-heterocyclyl” or “N-attached heterocyclyl” refers to a heterocyclyl radical as defined herein containing at least one nitrogen and where the point of attachment of the heterocyclyl radical to the rest of the molecule is through a nitrogen atom in the heterocyclyl radical. An N-heterocyclyl radical is optionally substituted as described herein for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“N-heteroaryl” refers to a heteroaryl radical as defined herein containing at least one nitrogen and where the point of attachment of the heteroaryl radical to the rest of the molecule is through a nitrogen atom in the heteroaryl radical. An N-heteroaryl radical is optionally substituted as described herein for heteroaryl radicals.

“Nitro” refers to the —NO2 radical.

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Oxa” or “Oxy” refers to the —O— radical.

“Oximo” refers to the ═N—OH radical.

“Oxo” refers to the ═O radical.

As used herein, the term “partially unsaturated” refers to a ring moiety that includes at least one double or triple bond between ring atoms but is not aromatic. The term “partially unsaturated” is intended to encompass rings having multiple sites of unsaturation, but is not intended to include aryl or heteroaryl moieties, as herein defined.

Any of the compounds of the present invention may be provided as a pharmaceutically acceptable salt.

The compounds of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} may be provided as pharmaceutically acceptable salts. “Pharmaceutically acceptable salt” includes both acid and base addition salts. A pharmaceutically acceptable salt of any one of the substituted pyrazolylpyridine derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

The compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} may be provided as pharmaceutically acceptable salts. “Pharmaceutically acceptable salt” includes both acid and base addition salts. A pharmaceutically acceptable salt of any one of the substituted pyrazolylpyridine derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts. “Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al, “Pharmaceutical Salts,” Journal of Pharmaceutical Science, 66: 1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al, supra.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism {see, e.g., Bundgard, H., Design of Prodrugs (1985), pp. 7-9, 21-24 (Elsevier, Amsterdam). A discussion of prodrugs is provided in Higuchi, T., et al, “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Solvates

The compound of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} may be provided in dissoluble or indissoluble forms together with a pharmaceutically acceptable solvent such as water, ethanol, and the like. Dissoluble forms may also include hydrated forms such as the mono-hydrate, the dihydrate, the hemihydrate, the trihydrate, the tetrahydrate, and the like.

The compound of Formulae I^{a}, I^{a1}, I^{a2}, I^{b}, I^{b2}, I^{b3}, I^{b3a}, I^{b3b}, II^{b3}, I^{b4}, I^{b5}, II^{b5}, I^{b6}, II^{b6}, I^{c} and I^{c2}

may be provided in dissoluble or indissoluble forms together with a pharmaceutically acceptable solvent such as water, ethanol, and the like. Dissoluble forms may also include hydrated forms such as the mono-hydrate, the dihydrate, the hemihydrate, the trihydrate, the tetrahydrate, and the like.

### Isotopic Variations

Elemental symbols and element names are used herein to include isotopes of the named elements. In particular one, some, or all hydrogens may be deuterium. Radioactive isotopes may be used, for instance to facilitate tracing the fate of the compounds or their metabolic products after administration.

A “stereoisomer” refers to a compound made up of the same atoms bonded by the same bonds but having different three-dimensional structures, which are not interchangeable. It is therefore contemplated that various stereoisomers and mixtures thereof and includes “enantiomers,” which refers to two stereoisomers whose molecular structures are nonsuperimposeable mirror images of one another

A “tautomer” refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. “Therapeutically effective amount” refers to an amount of a compound of the present invention that (i) treats the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. For cancer therapy, efficacy can, for example, be measured by assessing the time to disease progression (TTP) and/or determining the response rate (RR). In the case of immunological disorders, the therapeutic effective amount is an amount sufficient to decrease or alleviate an allergic disorder, the symptoms of an autoimmune and/or inflammatory disease, or the symptoms of an acute inflammatory reaction (e.g. asthma). In some embodiments, a therapeutically effective amount is an amount of a chemical entity described herein sufficient to significantly decrease the activity or number of drug tolerant or drug tolerant persisting cancer cells.

“Thioxo” refers to the ═S radical.

As used herein, “treatment” or “treating,” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “unsaturated”, as used herein, means that a moiety has one or more units of unsaturation.

“Pulse Dosing Regimen” refers to administering a KDM5 inhibitor to a patient for a first period of time and a second period of time. In one embodiment, the KDM5 inhibitor is administered at a higher dose in the first period of time followed by a lower dose in the second period of time. In another embodiment, the KDM5 inhibitor is administered at a lower dose in the first period of time followed by a higher dose at a second period of time. In one embodiment, the KDM5 inhibitor is administered at a first dose in the first period of time followed by a second dose at a second period of time. In one embodiment, the KDM5 inhibitor is administered at a first dose in the first period of time followed by a second dose at a second period of time wherein the first dose and second dose are equal. In one embodiment, the second period of time is at least 24 hours after the first period of time. In another embodiment, the second period of time is at least 48 hours after the first period of time. In another embodiment, the second period of time is at least 72 hours after the first period of time. In another embodiment, the second period of time is at least 96 hours after the first period of time. In another embodiment, the second period of time is at least 120 hours after the first period of time. In another embodiment, the second period of time is at least 144 hours after the first period of time. In another embodiment, the second period of time is at least 168 hours after the first period of time. In another embodiment, the second period of time is at least 192 hours after the first period of time. In another embodiment, the second period of time is between 120 and 144 hours after the first period of time. In another embodiment, the second period of time is between 144 and 168 hours after the first period of time. In another embodiment, the second period of time is between 168 and 192 hours after the first period of time.

It is understood that the divalent groups may be represented by the monovalent terms as defined above. For example alkylene terms such as methylene, ethylene, propylene, butylene, pentylene, hexylene, cyclopropylene, cyclobutylene, cyclopentylene, or cyclohexylene may be represented by alkyl terms such as methyl, ethyl, propyl, butyl, pentyl, hexyl, cyclopropyl, cyclobutyl, cyclopentyl, or cyclohexyl, respectively.

### KDM5 Inhibitor Compounds

In some embodiments of the invention, the KDM5 inhibitor is a compound of Formula I^{a}:

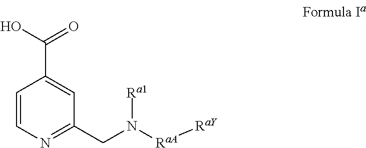

wherein:
R^{aA} is —CHR^{a2}C(O)—, C₁₋₈ alkylene, C₂₋₈ alkenylene, C₂₋₈ alkynylene, C₃₋₁₀ cycloalkylene, heterocyclylene, heteroarylene or arylene;
   wherein each alkylene, alkenylene, alkynylene, cycloalkylene, heterocyclylene, heteroarylene and arylene may optionally be substituted with one or more R^{a3};
R^{aY} is —H, —NR^{a6}R^{a7}, —OR^{a7}, C₁₋₈ alkyl, C₂₋₈ alkenyl, C₂₋₈ alkynyl, C₃₋₁₀ cycloalkyl, heterocyclyl, heteroaryl or aryl;
   wherein each alkyl, alkenyl, alkynyl, cycloalkyl, heterocyclyl, heteroaryl and aryl may optionally be substituted with one or more R^{a3} and may form a cyclic structure with R^{a2};
R^{a1} is —H, C₁₋₈ alkyl, C₂₋₈ alkenyl, C₂₋₈ alkynyl, or C₃₋₁₀ cycloalkyl:
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; or
   wherein each alkyl, alkenyl, alkynyl and cycloalkyl may be optionally substituted with one or more —H or C₁₋₄ alkyl; or
   wherein R^{a1} with —R^{aA}—R^{aY} forms a nitrogen containing optionally substituted heterocyclic group wherein the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl;
R^{a2} is —H, C₁₋₈ alkyl, C₂₋₈ alkenyl, C₂₋₈ alkynyl or C₃₋₁₀ cycloalkyl;
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl, and may form a cyclic structure with R^{aY}:
each R^{a3} is independently C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —R^{aZ}-heterocyclyl, —R^{aZ}-aryl, —R^{aZ}-heteroaryl, —R^{aZ}—NR^{a6}R^{a7}, —R^{aZ}—C(═O)—NR^{a6}R^{a7}, —R^{aZ}—NR^{a6}—C(═O)—R^{a7}, —R^{aZ}—C(═O)—R^{a7}, —R^{aZ}—OR^{a7}, halogen, —R^{aZ}—SR^{a7}, —R^{aZ}—SOR^{a7}, —R^{aZ}—SO₂R^{a}, —R^{aZ}—SO₂NR^{a6}R^{a7} or —R^{aZ}—COOR^{a7};
   wherein any heterocyclyl may be substituted with one or more R^{a4}; and
   wherein any heteroaryl and any aryl may be substituted with one or more R^{a5};
R^{aZ} is a single bond, C₁₋₄ alkylene, heterocyclylene or C₃₋₆ cycloalkylene:
each R^{a4} is independently C₁₋₆ alkyl, C₁₋₄ fluoroalkyl, C₁₋₄ hydroxyalkyl, C₁₋₄ alkoxy, C₃₋₁₀ cycloalkyl, —N(R^{a1})₂, carbamoyl or —OH:
each R^{a5} is independently C₁₋₆ alkyl, C₁₋₄ fluoroalkyl, C₁₋₄ hydroxyalkyl, C₁₋₄ alkoxy, C₃₋₆ cycloalkyl, —CN, —F, —Cl, —Br, carbamoyl or —OH;
each of R^{a6} and R^{a7} is independently —H, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-heteroaryl or —R^{aZ}-aryl;
   wherein each alkyl, alkenyl, alkynyl, cycloalkyl, heterocyclyl, heteroaryl and aryl may optionally be substituted with one or more independently selected R^{a8}; or
   wherein R^{a6} and R^{a7} may together with the N-atom to which they are attached form an N-heterocyclic ring optionally substituted with one or more independently selected R^{a8}:
each R^{a8} is independently C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9}, halogen, —CN, —R^{aZ}—SR^{a9}, —R^{aZ}—SOR^{a9}, —R^{aZ}—SO₂R^{a9} or —R^{aZ}—COOR^{a9};
   wherein each alkyl, alkenyl, alkynyl, cycloalkyl, heterocyclics, heteroaryl and aryl may optionally be substituted with one or more C₁₋₄ alkyl, C₁₋₄ fluoroalkyl, C₁₋₄ hydroxyalkyl, C₃₋₆ cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9}, halogen, —CN, —R^{aZ}—SR^{a9}, —R^{aZ}—SOR^{a9}, —R^{aZ}—SO₂R^{a9} or —R^{aZ}—COOR^{a9};
      wherein any heterocyclyl may be further substituted with one or more R^{a4} as defined above, and
      wherein any heteroaryl and any aryl may be further substituted with one or more R^{a5} as defined above;
each R^{a9} is independently —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-aryl or —R^{aZ}-heteroaryl;
   wherein any heterocyclyl may be substituted with one or more R^{a4} as defined above; and
   wherein any heteroaryl and any aryl may be substituted with one or more R^{a5} as defined above; and
each of R^{a10} and R^{a11} is independently —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl or aryl;
   wherein any heterocyclyl may be substituted with one or more R^{a4} as defined above; and
   wherein any heteroaryl and any aryl may be substituted with one or more R^{a5} as defined above; or
   wherein R^{a10} and R^{a11} may together with the N-atom to which they are attached form an N-heterocyclic ring optionally substituted with one or more R^{a4} as defined above.

A prodrug of Formula I^{a1} may be in the form:

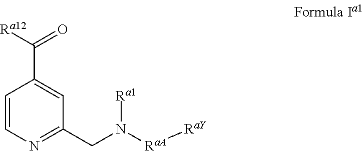

wherein:
R^{a12} is of the form (R^{a13})₂N- or of the form R^{a13}O—, wherein each R^{a13} independently may be selected from C1-8 alkyl, C2-8 alkenyl, C2-8 alkynyl, C3-10 cycloalkyl, and aryloxy wherein each alkyl, alkenyl, alkynyl, cycloalkyl and aryloxy may be optionally substituted with one or more selected from —OH, aryl, C1-6 alkoxy, heteroaryl, aryloxy, heteroaryloxy, F, a sulphonamide moiety, and C3-6 cycloalkyl; and one Ra13 in (Ra13)2N-may be —H;

In some embodiments of the invention, the KDM5 inhibitor is a prodrug of a compound of Formula I^{a} having Formula I^{a1}

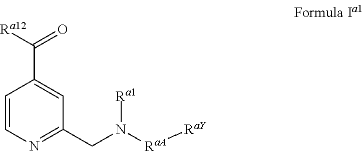

wherein:
R^{a12} is of the form (R^{a13})₂N- or of the form R^{a13}O—, wherein each R^{a13} independently may be selected from C1-8 alkyl, C2-8 alkenyl, C2-8 alkynyl, C3-10 cycloalkyl, and aryloxy wherein each alkyl, alkenyl, alkynyl, cycloalkyl and aryloxy may be optionally substituted with one or more selected from —OH, aryl, C1-6 alkoxy, heteroaryl, aryloxy, heteroaryloxy, F, a sulphonamide moiety, and C3-6 cycloalkyl; and one Ra13 in (Ra13)2N-may be, and preferably is, —H.

Another embodiment provides a compound Formula I^{a1}:

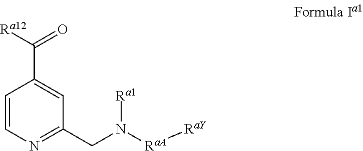

wherein:
R^{a12} is of the form (R^{a13})₂N- or of the form R^{a13}O—, wherein each R^{a13} independently may be selected from C1-8 alkyl, C2-8 alkenyl, C2-8 alkynyl, C3-10 cycloalkyl, and aryloxy wherein each alkyl, alkenyl, alkynyl, cycloalkyl and aryloxy may be optionally substituted with one or more selected from —OH, aryl, C1-6 alkoxy, heteroaryl, aryloxy, heteroaryloxy, F, a sulphonamide moiety, and C3-6 cycloalkyl; and one Ra13 in (Ra13)2N-may be H;
R^{aA} is —CHR^{a2}C(O)—, C₁₋₈ alkylene, C₂₋₈ alkenylene, C₂₋₈ alkynylene, C₃₋₁₀ cycloalkylene, heterocyclylene, heteroarylene or arylene;
   wherein each alkylene, alkenylene, alkynylene, cycloalkylene, heterocyclylene, heteroarylene and arylene may optionally be substituted with one or more R^{a3}:
R^{aY} is —H, —NR^{a6}R^{a7}, —OR^{a7}, C₁₋₈ alkyl, C₂₋₈ alkenyl, C₂₋₈ alkynyl, C₃₋₁₀ cycloalkyl, heterocyclyl, heteroaryl or aryl;
   wherein each alkyl, alkenyl, alkynyl, cycloalkyl, heterocyclyl, heteroaryl and aryl may optionally be substituted with one or more R^{a3} and may form a cyclic structure with R^{a2};
R^{a1} is —H, C₁₋₈ alkyl, C₂₋₈ alkenyl, C₂₋₈ alkynyl, or C₃₋₁₀ cycloalkyl;
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; or
   wherein each alkyl, alkenyl, alkynyl and cycloalkyl may be optionally substituted with one or more —H or C₁₋₄ alkyl; or
   wherein R^{a1} with —R^{aA}—R^{aY} forms a nitrogen containing optionally substituted heterocyclic group wherein the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl;
R^{a2} is —H, C₁₋₈ alkyl, C₂₋₈ alkenyl, C₂₋₈ alkynyl or C₃₋₁₀ cycloalkyl;
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl, and may form a cyclic structure with R^{aY};
each R^{a3} is independently C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —R^{aZ}-heterocyclyl, —R^{aZ}-aryl, —R^{aZ}-heteroaryl, —R^{aZ}—NR^{a6}R^{a7}, —R^{aZ}—C(═O)—NR^{a6}R^{a7}, —R^{aZ}—NR^{a6}—C(═O)—R^{a7}, —R^{aZ}—C(═O)—R^{a7}, —R^{aZ}—OR^{a7}, halogen, —R^{aZ}—SR^{a7}, —R^{aZ}—SOR^{a7}, —R^{aZ}—SO₂R^{a7}, —R^{aZ}—SO₂NR^{a6}R^{a7} or —R^{aZ}—COOR^{a7};
   wherein any heterocyclyl may be substituted with one or more R^{a4}; and
   wherein any heteroaryl and any aryl may be substituted with one or more R^{a5};
R^{aZ} is a single bond, C₁₋₄ alkylene, heterocyclylene or C₃₋₆ cycloalkylene;
each R^{a4} is independently C₁₋₆ alkyl, C₁₋₄ fluoroalkyl, C₁₋₄ hydroxyalkyl, C₁₋₄ alkoxy, C₃₋₁₀ cycloalkyl, —N(R^{a1})₂, carbamoyl or —OH;
each R^{a5} is independently C₁₋₆ alkyl, C₁₋₄ fluoroalkyl, C₁₋₄ hydroxyalkyl, C₁₋₄ alkoxy, C₃₋₁₀ cycloalkyl, —CN, —F, —Cl, —Br, carbamoyl or —OH;
each of R^{a6} and R^{a7} is independently —H, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-heteroaryl or —R^{aZ}-aryl;
   wherein each alkyl, alkenyl, alkynyl, cycloalkyl, heterocyclyl, heteroaryl and aryl may optionally be substituted with one or more independently selected R^{a8}; or
   wherein R^{a6} and R^{a7} may together with the N-atom to which they are attached form an N-heterocyclic ring optionally substituted with one or more independently selected R^{a8};
each R^{a8} is independently C₁₋₆ alkyl, C₁₋₄ fluoroalkyl, C₁₋₄ hydroxyalkyl, C₂₋₆ alkenyl, C₂₋₆ alkynyl, C₃₋₁₀ cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9}, halogen, —CN, —R^{e}—SR^{a9}, —R^{aZ}—SOR^{a9}, —R^{aZ}—SO₂R^{a9} or —R^{aZ}—COOR^{a9};
   wherein each alkyl, alkenyl, alkynyl, cycloalkyl, heterocyclics, heteroaryl and aryl may optionally be substituted with one or more C₁₋₄ alkyl, C₁₋₄ fluoroalkyl, C₁₋₄ hydroxyalkyl, C₃₋₆ cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9}, halogen, —CN, —R^{aZ}—SR^{a9}, —R^{aZ}—SOR^{a9}, —R^{aZ}—SO₂R^{a9} or —R^{aZ}—COOR^{a9};
      wherein any heterocyclyl may be further substituted with one or more R^{a4} as defined above, and
      wherein any heteroaryl and any aryl may be further substituted with one or more R^{a5} as defined above;
each R^{a9} is independently —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-aryl or —R^{aZ}-heteroaryl;
   wherein any heterocyclyl may be substituted with one or more R⁴ as defined above; and
   wherein any heteroaryl and any aryl may be substituted with one or more R^{a5} as defined above; and
each of Rₐ₁₀ and Rₐ₁₁ is independently —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl or aryl;
   wherein any heterocyclyl may be substituted with one or more R^{a4} as defined above; and
   wherein any heteroaryl and any aryl may be substituted with one or more R^{a5} as defined above; or
wherein R^{a10} and R^{a11} may together with the N-atom to which they are attached form an N-heterocyclic ring optionally substituted with one or more R^{a4} as defined above;
or a pharmaceutically acceptable salt thereof.

Non-exhaustive examples of Formula I^{a} include:

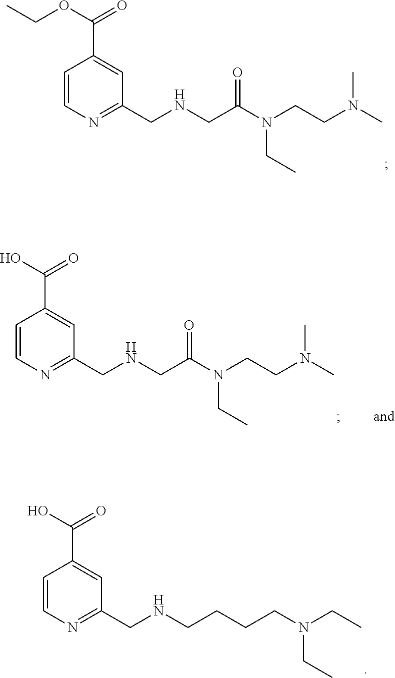 [SMILES: CCOC(=O)c1ccnc(CNCC(=O)N(CC)CCN(C)C)c1 ; CCN(CCN(C)C)C(=O)CNCc1cc(C(=O)O)ccn1 ; CCN(CC)CCCCNCc1cc(C(=O)O)ccn1]

Non-exhaustive examples of Formula I^{a1} include:

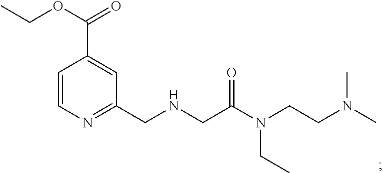 [SMILES: CCOC(=O)c1ccnc(CNCC(=O)N(CC)CCN(C)C)c1]

or a pharmaceutically acceptable salt thereof.

In some embodiments of the invention, the KDM5 inhibitor is a compound of Formula I^{a2}:

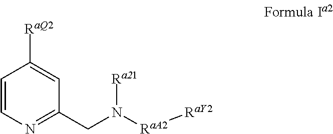

wherein:
R^{aQ2} is —CH═NR^{a32}, —R^{a38}, —CH₂NHR^{a33}, —CH═O, —CH(OR^{a37})₂ or C(O)OR^{a23};
R^{aA2} is —CHR^{a22}C(O)—, C₁₋₈ alkylene, C₂₋₈ alkenylene, C₂₋₈ alkynylene, C₃₋₁₀ cycloalkylene, heterocyclylene, heteroarylene or arylene;
   wherein each alkylene, alkenylene, alkynylene, cycloalkylene, heterocyclylene, heteroarylene and arylene may optionally be substituted with one or more R^{a23};
   with the proviso that when R^{aQ2} is —CH═O, R^{aA2} is not alkynylene;
R^{aY2} is —H, —NR^{a26}R^{a27}, —OR^{a27}, C₁₋₈ alkyl, C₂₋₈ alkenyl, C₂₋₈ alkynyl, C₃₋₁₀ cycloalkyl, heterocyclyl, heteroaryl or aryl;
   wherein each alkyl, alkenyl, alkynyl, cycloalkyl, heterocyclyl, heteroaryl and aryl may optionally be substituted with one or more R^{a23} and may form a cyclic structure with R^{a22};
   with the proviso that when R^{aQ2} is —CH═O, R^{aY2} is not alkynyl;
R^{a21} is —H, C₁₋₈ alkyl, C₂₋₈ alkenyl, C₂₋₈ alkynyl or C₃₋₁₀ cycloalkyl;
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; or
   wherein each alkyl, alkenyl, alkynyl and cycloalkyl may be optionally substituted with one or more —H or C₁₋₄ alkyl; or
   wherein R^{a21} with —R^{aA2}—R^{aY2} forms a nitrogen containing optionally substituted heterocyclic group:
      wherein the optional substitution may be C₁₋₈ alkyl, C₂₋₈ alkenyl, C₂₋₈ alkynyl or C₃₋₁₀ cycloalkyl;
      wherein each alkyl, alkenyl, alkynyl and cycloalkyl may be optionally substituted with one or more —OH, aryl, C₁₋₄ alkoxy, heteroaryl, aryloxy, heteroaryloxy, F or C₃₋₆ cycloalkyl;
R^{a22} is —H, C₁₋₈ alkyl, C₂₋₈ alkenyl, C₂₋₈ alkynyl or C₃₋₁₀ cycloalkyl;
   wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; and
   may form a cyclic structure with R^{aY2};
each R^{a23} is independently C₁₋₆ alkyl, C₁₋₄ fluoroalkyl, C₁₋₄ hydroxyalkyl, C₂₋₆ alkenyl, C₂₋₆ alkenyl, C₃₋₁₀ cycloalkyl, —R^{aZ2}-heterocyclyl, —R^{aZ2}-aryl, —R^{aZ2}-heteroaryl, —R^{aZ2}—NR^{a26}R^{a27}, —R^{aZ2}—C(═O)—NR^{a26}R^{a27}, —R^{aZ2}—NR^{a26}—C(═O)—R^{a27}, —R^{aZ2}—C(═O)—R^{a27}, —R^{aZ2}—OR^{a27}, halogen, —R^{aZ2}—SR^{a27}, —R^{aZ2}—SOR^{a27}, —R^{aZ2}—SO₂R^{a27}, —R^{aZ2}—SO₂NR^{a26}R^{a27} or —R^{aZ2}—COOR^{a27};
   wherein any heterocyclyl may be substituted with one or more R^{a24}; and
   wherein any heteroaryl and any aryl may be substituted with one or more R^{a25};
R^{aZ2} is a single bond, C₁₋₄ alkylene, heterocyclylene or C₃₋₆ cycloalkylene;
each R^{a24} is independently C₁₋₆ alkyl, C₁₋₄ fluoroalkyl, C₁₋₄ hydroxyalkyl, C₁₋₄ alkoxy, C₃₋₁₀ cycloalkyl, —N(R^{a21})₂, carbamoyl or —OH;
each R^{a25} is independently C₁₋₆ alkyl, C₁₋₄ fluoroalkyl, C₁₋₄ hydroxyalkyl, C₁₋₄ alkoxy, C₃₋₆ cycloalkyl, —CN, —F, —Cl, —Br, carbamoyl or —OH;
each of R^{a26} and R^{a27} is independently C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —R^{aZ2}-heterocyclyl, —R^{aZ2}-heteroaryl or —R^{aZ2}-aryl;
   wherein each alkyl, alkenyl, alkynyl, cycloalkyl, heterocyclyl, heteroaryl and aryl may optionally be substituted with one or more independently selected R^{a28}; or wherein R^{a26} and R^{a27} may together with the N-atom to which they are attached form an N-heterocyclic ring optionally substituted with one or more independently selected R^{a28};
each R^{a28} is independently C₁₋₄ alkyl, C₁₋₄ fluoroalkyl, C₁₋₄ hydroxyalkyl, C₂₋₆ alkenyl, C₂₋₆ alkynyl, C₃₋₁₀ cycloalkyl, —R^{aZ2}-heterocyclyl, —R^{aZ2}-heteroaryl, —R^{aZ2}-aryl, —R^{aZ2}—NR^{a30}R^{a31}, —R^{aZ2}—C(═O)—NR^{a30}R^{a31}, —R^{aZ2}—OR^{a29}, halogen, —CN, —R^{aZ2}—SR^{a29}, —R^{aZ2}—SOR^{a29}, —R^{aZ2}—SO₂R^{a29} or —R^{aZ2}—COOR^{a29};
   wherein each alkyl, alkenyl, alkynyl, cycloalkyl, heterocyclics, heteroaryl and aryl may optionally be substituted with one or more C₁₋₄ alkyl, C₁₋₄ fluoroalkyl, C₁₋₄ hydroxyalkyl, C₃₋₆ cycloalkyl, —R^{aZ2}-heterocyclyl, —R^{aZ2}-heteroaryl, —R^{a2}-aryl, —R^{aZ2}—NR^{a30}R^{a31}, —R^{aZ2}—C(═O)—NR^{a30}R^{a31}, —R^{aZ2}—OR^{a29}, halogen, —CN, —R^{aZ2}—SR^{a29}, —R^{aZ2}—SOR^{a29}, —R^{aZ2}—SO₂R^{a9} or —R^{aZ2}—COOR^{a29};
   wherein any heterocyclyl may be further substituted with one or more R^{a24} as defined above; and
   wherein any heteroaryl and any aryl may be further substituted with one or more R^{a25} as defined above, and
each R^{a29} is independently —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —R^{aZ2}-heterocyclyl, —R^{aZ2}-aryl or —R^{aZ2}-heteroaryl;
   wherein any heterocyclyl may be substituted with one or more R^{a24} as defined above; and
   wherein any heteroaryl and any aryl may be substituted with one or more R^{a25} as defined above;
each of R^{a30} and R^{a31} is independently —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl or aryl;
   wherein any heterocyclyl may be substituted with one or more R^{a24} as defined above; and
   wherein any heteroaryl and any aryl may be substituted with one or more R^{a25} as defined above; or
   wherein R^{a30} and R^{a31} may together with the N-atom to which they are attached form an optionally 5 to 7 membered, N-heterocyclic ring optionally substituted with one or more R^{a24} as defined above;
with the proviso that R^{aY2} is not H when R^{aA2} is —CH₂—;
R^{a32} is C₁₋₁₀ alkyl, C₂₋₁₀ alkenyl, C₂₋₁₀ alkynyl, C₃₋₁₀ cycloalkyl, —R^{aZ2}-heterocyclyl, —R^{aZ2}-aryl, —R^{aZ2}-heteroaryl, —R^{aZ2}—NR^{a26}R^{a27}, —R^{aZ2}—C(═O)—NR^{a26}R^{a27}, —R^{aZ2}—NR^{a26}—C(═O)—R^{a27}, —R^{aZ2}—C(═O)—R^{a27}, —R^{aZ2}—OR^{a27}, halogen, —R^{aZ2}—SR^{a27}, —R^{aZ2}—SOR^{a27}, —R^{aZ2}—SO₂R^{a27} or —R^{aZ2}—COOR^{a27};
   wherein each alkyl, alkenyl, alkynyl, cycloalkyl, heterocyclyl, heteroaryl and aryl may optionally be substituted with one or more R^{a23}; R^{a33} is hydrogen, —C(O)R^{a27}, —C(O)C(O)R^{a27}, —C(O)C(O)OR^{a27}, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl, C₃₋₁₀ cycloalkyl, —R^{aZ2}-heterocyclyl or —R^{aZ2}-monocyclic-heteroaryl;
   wherein each alkyl, alkenyl, alkynyl, cycloalkyl, heterocyclyl and monocyclic-heteroaryl may optionally be substituted with one or more independently selected R^{a28}; or
   wherein each alkyl, alkenyl, alkynyl, cycloalkyl, heterocyclyl and monocyclic-heteroaryl may optionally be substituted with one or more —CR^{a34}R^{a35}—NR^{a26}R^{a27}, —CR^{a34}R^{a35}CN or —CR^{a34}R^{a35}OR^{a27};
   wherein each of R^{a34} and R^{a35} is independently —H, C₁₋₈ alkyl, C₂₋₈ alkenyl, C₂₋₈ alkynyl, C₃₋₁₀ cycloalkyl, heterocyclyl, heteroaryl and aryl; or
   wherein R^{a34} and R^{a35} together with the intervening carbon atom may designate a C₃₋₁₀ cycloalkyl or C₅₋₁₀-cycloalkenyl ring, which alkyl, alkenyl, alkynyl, cycloalkyl (ring), cycloalkenyl ring, heterocyclyl, heteroaryl and aryl may optionally be substituted with one or more R^{a23};
R^{a38} is an 1,3-diaza-C₅₋₇-cycloalk-2-yl group which is N-substituted with R^{a36} and optionally further substituted with one or more R^{a23}, and optionally containing one or two oxo groups; a 1,3-thiaza-C₅₋₇-cycloalk-2-yl group which is N-substituted with R^{a36} and optionally further substituted with one or more R^{a23} and optionally containing one or two oxo groups; an 1,3-oxaza-C₅₋₇-cycloalk-2-yl group which is N-substituted with R^{a36} and optionally further substituted with one or more R^{a23}, and optionally containing one or two oxo groups, wherein in all three instances two R^{a23}'s on the same carbon atom may together form a spiro group;
R^{a36} is hydrogen, —C(O)R^{a27}, —C(O)C(O)R²⁷ or —C(O)C(O)OR²⁷;
each R^{a37} independently is R^{a23}; or
   wherein two R^{a37} substituents together with the intervening —O—CH(−)—O— may form a heterocyclyl optionally substituted with one or more R^{a23} and containing up to two oxo groups;
or an isomer or a mixture of isomers thereof, or a pharmaceutically acceptable salt, or solvate or prodrug thereof.

In some embodiments of Formula I^{a2}, R^{aQ2} is a group that is converted to —COOH or COO⁻ upon administration of said compound to a human, provided that R^{aQ2} is not an amide or an ester of such a —COOH group.

Non-exhaustive examples of Formula I^{a2} include:

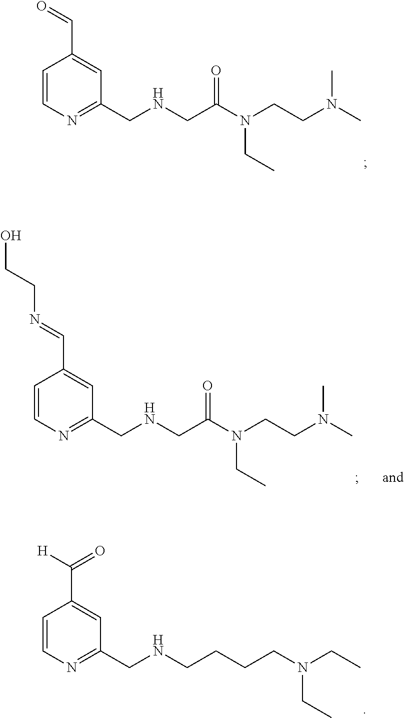 [SMILES: CCN(CCN(C)C)C(=O)CNCc1cc(C=O)ccn1 ; CCN(CCN(C)C)C(=O)CNCc1cc(/C=N/CCO)ccn1 ; CCN(CC)CCCCNCc1cc(C=O)ccn1]

In some embodiments of the invention, the KDM5 inhibitor is a compound of Formula I^{b}:

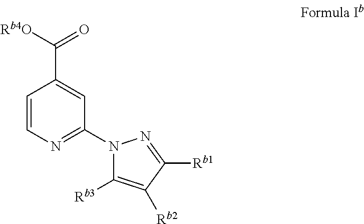

or a tautomer, stereoisomer, geometric isomer, N-oxide, or a pharmaceutically acceptable salt thereof;
   wherein:
R^{b1} is hydrogen, halogen, —OH, —OR^{b5}, —N(R^{b5})₂, alkyl, carbocyclyl, heterocyclyl, aryl, heteroaryl, carbocyclylalkyl, heterocyclylalkyl, aralkyl or heteroarylalkyl;
R^{b2} is hydrogen, —OH, —OR^{b5}, —N(R^{b5})₂, alkyl, carbocyclyl, heterocyclyl, aryl, heteroaryl, carbocyclylalkyl, heterocyclylalkyl, aralkyl, hydroxyalkyl or heteroarylalkyl;
R^{b3} is hydrogen, halogen, —OH, —OR^{b5}, —N(R^{b5})₂, alkyl, carbocyclyl, heterocyclyl, aryl, heteroaryl, carbocyclylalkyl, heterocyclylalkyl, aralkyl or heteroarylalkyl;
R^{b4} is hydrogen or alkyl;
each R^{b5} is independently hydrogen, alkyl, carbocyclyl, heterocyclyl, aryl, heteroaryl, carbocyclylalkyl, heterocyclylalkyl, aralkyl or heteroarylalkyl;
   wherein each alkyl, carbocyclyl, heterocyclyl, aryl, heteroaryl, carbocyclylalkyl, heterocyclylalkyl, aralkyl or heteroarylalkyl may be optionally substituted with one or two halogen: F, Cl, Br, and I, or alkyl
with the provisos:
if R^{b2} and R^{b3} are both hydrogen, then R^{b2} is not hydrogen, methyl, trifluoromethyl, isopropyl or cyclopropyl; or
if R^{b1} and R^{b3} are both hydrogen, then R^{b2} is not methyl or trifluoromethyl; or
if R^{b1} and R^{b3} are both methyl, then R^{b2} is not hydrogen, methyl or ethyl; or
if R^{b1} and R^{b2} are hydrogen, then R^{b3} is not

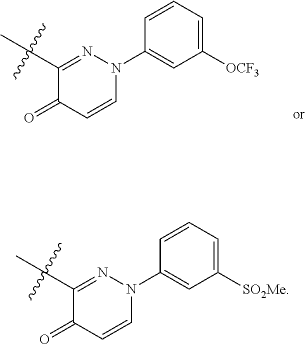 [SMILES: *c1nn(-c2cccc(OC(F)(F)F)c2)ccc1=O ; *c1nn(-c2cccc(S(C)(=O)=O)c2)ccc1=O]

Non-exhaustive examples of Formula I^{b} include:

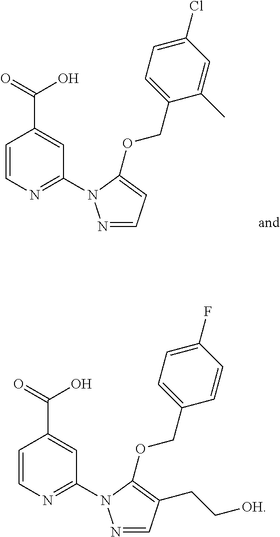 [SMILES: Cc1cc(Cl)ccc1COc1ccnn1-c1cc(C(=O)O)ccn1 ; O=C(O)c1ccnc(-n2ncc(CCO)c2OCc2ccc(F)cc2)c1]

In some embodiments of the invention, the KDM5 inhibitor is a compound of Formula I^{b2}:

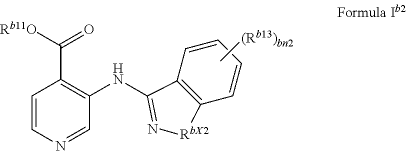

or a tautomer, stereoisomer, geometric isomer, N-oxide, or a pharmaceutically acceptable salt thereof:
wherein:
R^{bX2} is O or NR^{b15};
R^{b11} is hydrogen or alkyl;
each R^{b11} is independently hydroxy, halogen, cyano, NH₂, NHR^{b14}, N(R^{b14})₂, NHC(O)R^{b14}, NHC(O)OR^{b14}, NHC(O)NHR^{b14}, NHC(O)N(R^{b14})₂, NHS(O)₂R^{b14}, NR^{b14}C(O)R^{b14}, NR^{b14}C(O)OR^{b14}, NR^{b14}C(O)NHR^{b14}, NR^{b14}C(O)N(R^{b14})₂, NR^{b14}S(O)₂R^{b14}, alkyl, alkenyl, alkynyl, alkoxy, aryl, aryloxy, aralkyl, carbocyclyl, heterocyclyl, heteroaryl, carbocyclylalkyl, heterocyclylalkyl or heteroarylalkyl;
each R^{b14} is independently alkyl, aryl, aralkyl, carbocyclyl, heterocyclyl, heteroaryl, carbocyclylalkyl, heterocyclylalkyl or heteroarylalkyl;
R^{b15} is alkyl, alkenyl, alkynyl, aryl, aralkyl, carbocyclyl, heterocyclyl, heteroaryl, carbocyclylalkyl, heterocyclylalkyl or heteroarylalkyl;
   wherein each alkyl, alkenyl, and alkynyl is optionally substituted with a heterocyclyl;
      wherein each heterocyclyl is optionally substituted with one, two, or three halogens; and
bn2 is an integer 0, 1, 2, 3, or 4.

Non-exhaustive examples of Formula I^{b2} include:

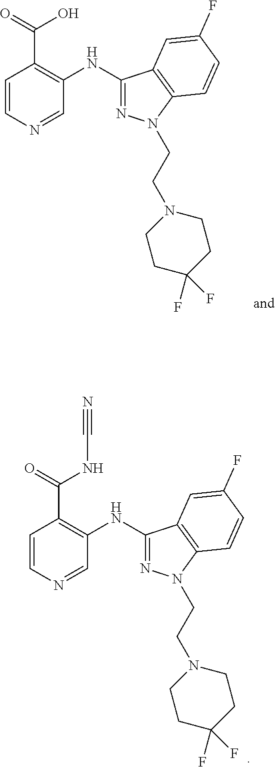 [SMILES: O=C(O)c1ccncc1Nc1nn(CCN2CCC(F)(F)CC2)c2ccc(F)cc12 ; N#CNC(=O)c1ccncc1Nc1nn(CCN2CCC(F)(F)CC2)c2ccc(F)cc12]

In some embodiments of the invention, the KDM5 inhibitor is a compound of Formula II^{b2}:

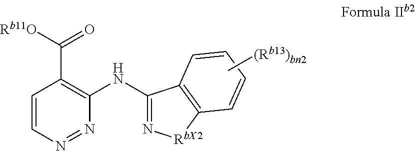

or a tautomer, stereoisomer, geometric isomer, N-oxide, or a pharmaceutically acceptable salt thereof:
wherein:
R^{bX2} is O or NR^{b15};
R^{b11} is hydrogen or alkyl;
each R^{b13} is independently hydroxy, halogen, cyano, NH₂, NHR^{b14}, N(R^{b14})₂, NHC(O)R^{b14}, NHC(O)OR^{b14}, NHC(O)NHR^{b14}, NHC(O)N(R^{b14})₂, NHS(O)R^{b14}, NR^{b14}C(O)R^{b14}, NR^{b14}C(O)OR^{b14}, NR^{b14}C(O)NHR^{b14}, NR^{b14}C(O)N(R^{b14})₂, NR^{b14}S(O)₂R^{b14}, alkyl, alkenyl, alkynyl, alkoxy, aryl, aryloxy, aralkyl, carbocyclyl, heterocyclyl, heteroaryl, carbocyclylalkyl, heterocyclylalkyl or heteroarylalkyl;
each R^{b14} is independently alkyl, aryl, aralkyl, carbocyclyl, heterocyclyl, heteroaryl, carbocyclylalkyl, heterocyclylalkyl or heteroarylalkyl;
R^{b15} is alkyl, alkenyl, alkynyl, aryl, aralkyl, carbocyclyl, heterocyclyl, heteroaryl, carbocyclylalkyl, heterocyclylalkyl or heteroarylalkyl; and
bn2 is an integer 0, 1, 2, 3, or 4.

In some embodiments of the invention, the KDM5 inhibitor is a compound of Formula I^{b3}:

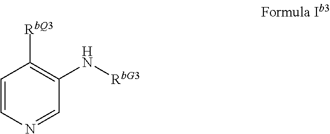

or a pharmaceutically acceptable salt thereof:
wherein:
R^{bQ3} is —CO₂R^{b20}, —C(O)N(H)CN, —C(O)N(H)OH or tetrazolyl;
R^{b20} is hydrogen or optionally substituted alkyl;
R^{bG3} is —R^{bX3}—R^{bY3};
   R^{bX3} is —C₁ alkylene;
   R^{bY3} is optionally substituted tetralinyl, optionally substituted tetrahydroquinolinyl, substituted pyridyl, optionally substituted naphthyl, optionally substituted indolyl, optionally substituted benzofuranyl, optionally substituted adamantyl or optionally substituted indanyl.

Non-exhaustive examples of Formula I^{b3} include:

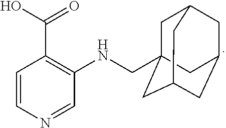 [SMILES: O=C(O)c1ccncc1NCC12CC3CC(CC(C3)C1)C2]

In further embodiments of the invention, the KDM5 inhibitor is a compound of Formula I^{b3}:

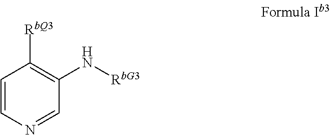

or a pharmaceutically acceptable salt thereof,
wherein:
R^{bQ3} is —CO₂R^{b20}, —C(O)N(H)CN, —C(O)N(H)OH or tetrazolyl;
R^{b20} is hydrogen or optionally substituted alkyl;
R^{bG3} is —R^{bX3}—R^{bY3};
   R^{bX3} is —C₁ alkylene;
   R^{bY3} is phenyl substituted with alkenyl, alkynyl, fluoro, chloro, fluoroalkyl, nitro, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b22}—OR^{b21}, —R^{b22}—OC(O)—R^{b21}, —R^{b22}—OC(O)—OR^{b21}, —R^{b22}—OC(O)N(R^{b21})₂, —R^{b22}—N(R^{b21})₂, —R^{b22}—C(O)R^{b21}, —R^{b22}—C(O)OR^{b21}, —R^{b22}—O—R^{b23}—C(O)N(R^{b21})₂, —R^{b22}—N(R^{b21})C(O)OR^{b21}, —R^{b22}—N(R^{b21})C(O)R^{b21}, —R^{b22}—N(R^{b21})S(O)_{bt3}R^{b21}, —R^{b22}—S(O)_{bt3}OR^{b21}, —R^{b22}—S(O)_{bt3}R^{b21} or —R^{b22}—S(O)_{bt3}N(R^{b1})₂;
      wherein:
      each R^{b21} is independently hydrogen, alkyl, fluoroalkyl, cycloalkyl, cycloalkylalkyl, aryl, aralkyl, heterocyclyl, heterocyclylalkyl, heteroaryl or heteroarylalkyl,
      each R^{b22} is independently a direct bond or a straight or branched alkylene or alkenylene chain;
      each R^{b23} is a straight or branched alkylene or alkenylene chain; and
      bt3 is 1 or 2.

In further embodiments of the invention, the KDM5 inhibitor is a compound of Formula I^{b3}:

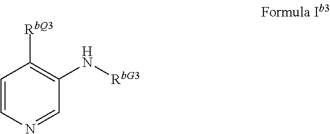

or a pharmaceutically acceptable salt thereof;
wherein:
R^{bQ3} is —CO₂R^{b20}, —C(O)N(H)CN, —C(O)N(H)OH or tetrazolyl;
R^{b20} is hydrogen or optionally substituted alkyl;
R^{bG3} is —R^{bX3}—R^{bY3};
   R^{bX3} is —C₁ alkylene;
   R^{bY3} is optionally substituted tetralinyl, optionally substituted chromanyl, optionally substituted tetrahydroquinolinyl, optionally substituted benzofuranyl, optionally substituted 2,3-dihydrobenzofuranyl, optionally substituted 2,3-dihydrobenzo[b][1,4]dioxinyl, optionally substituted naphthyl, optionally substituted indolyl, optionally substituted 1,2-dihydronaphthyl, optionally substituted indanyl or optionally substituted thiochromanyl.

In further embodiments of the invention, Formula I^{b3} or a pharmaceutically acceptable salt thereof, has the structure of Formula I^{b3a}:

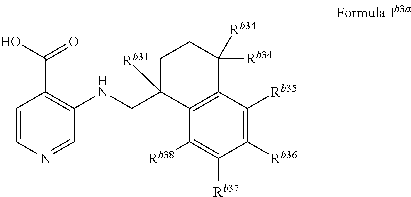

wherein:
R^{b31} is hydrogen, methyl, or —OH;
each R^{b34} is independently hydrogen, fluoro or methyl; and
R^{b35}, R^{b36}, R^{b37} and R^{b38} are each independently hydrogen, halogen, —OH, —CN, optionally substituted C₁₋₆ alkyl, optionally substituted C₁₋₆ alkoxy, optionally substituted C₃₋₇ carbocyclyl, optionally substituted C₃₋₇ carbocyclyloxy, optionally substituted C₄₋₁₂ carbocyclylalkyl, optionally substituted C₄₋₁₂ carbocyclylalkoxy, optionally substituted C₁₋₆ alkynyl, optionally substituted C₁₋₆ alkenyl, optionally substituted C₆₋₁₀ aryl, optionally substituted C₆₋₁₀ aryloxy, optionally substituted C₆₋₁₀ aryl-S—, optionally substituted C₇₋₁₄ aralkoxy, optionally substituted heteroaryl or optionally substituted heteroaryloxy.

In further embodiments of the invention, Formula I^{b3} or a pharmaceutically acceptable salt thereof, has the structure of Formula I^{b3b}:

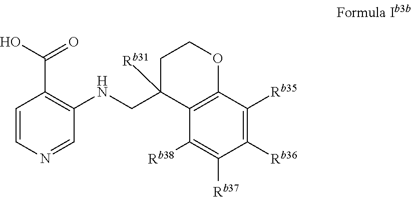

wherein
R^{b31} is hydrogen, methyl or —OH; and
R^{b35}, R^{b36}, R^{b37} and R^{b38} are each independently hydrogen, halogen, —OH, —CN, optionally substituted C₁₋₆ alkyl, optionally substituted C₁₋₆ alkoxy, optionally substituted C₃₋₇ carbocyclyl, optionally substituted C₃₋₇ carbocyclyloxy, optionally substituted C₄₋₁₂ carbocyclylalkyl, optionally substituted C₄₋₁₂ carbocyclylalkoxy, optionally substituted C₁₋₆ alkynyl, optionally substituted C₁₋₆ alkenyl, optionally substituted C₆₋₁₀ aryl, optionally substituted C₆₋₁₀ aryloxy, optionally substituted C₆₋₁₀ aryl-S—, optionally substituted C₇₋₁₄ aralkoxy, optionally substituted heteroaryl or optionally substituted heteroaryloxy.

In some embodiments of the invention, the KDM5 inhibitor is a compound of Formula II^{b3}:

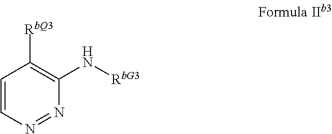

or a pharmaceutically acceptable salt thereof;
wherein:
R^{bQ3} is —CO₂R^{b20}, —C(O)N(H)CN, —C(O)N(H)OH or tetrazolyl;
R^{b20} is hydrogen or optionally substituted alkyl;
R^{bG3} is —R^{bX3}—R^{bY3};
   R^{bX3} is —C₁ alkylene;
   R^{bY3} is carbocyclyl, heterocyclyl, aryl or heteroaryl;
with the proviso that R^{bG3} is not

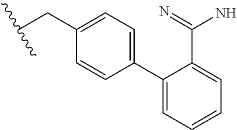 [SMILES: *Cc1ccc(-c2ccccc2C(=N)N)cc1]

In some embodiments of the invention, the KDM5 inhibitor is a compound of Formula I^{b4}:

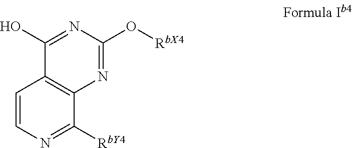

or a pharmaceutically acceptable salt thereof;
wherein:
R^{bX4} is alkyl, or —R^{bL4}—R^{b41}:
   R^{bL4} is a bond or C₁₋₆ alkylene;
   R^{b41} is carbocyclyl, aryl, heterocyclyl or heteroaryl;
   wherein each heteroaryl is optionally substituted with an optionally substituted aralkyl;
R^{bY4} is hydrogen or

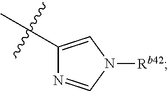

and
R^{b42} is alkyl, heterocyclyl, heterocyclylalkyl, or carbocyclylalkyl.

Non-exhaustive examples of Formula I^{b4} include:

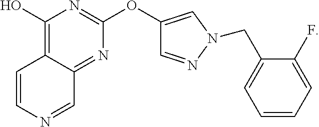 [SMILES: Oc1nc(Oc2cnn(Cc3ccccc3F)c2)nc2cnccc12]

In some embodiments of the invention, the KDM5 inhibitor is a compound of Formula I^{b5}:

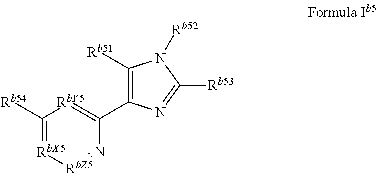

or a pharmaceutically acceptable salt thereof;
wherein:
R^{bX5} is CH, COH or N;
R^{bY5} is CH or N;
R^{bZ5} is CH or N;
R^{b51} is hydrogen, halogen, —OH, —OR^{b55}, —N(R^{b55})₂, alkyl, carbocyclyl, heterocyclyl, aryl, heteroaryl, carbocyclylalkyl, heterocyclylalkyl, aralkyl or heteroarylalkyl:
R^{b52} is alkyl, carbocyclyl, heterocyclyl, aryl, heteroaryl, carbocyclylalkyl, heterocyclylalkyl, aralkyl or heteroarylalkyl;
R^{b53} is hydrogen, halogen, —OH, —NH₂, —NH(C₁₋₃ alkyl) or C₁₋₃ alkyl;
R^{b54} is —CO₂H, —CO₂R^{b56}, —C(O)N(H)CN, —C(O)N(H)OH or tetrazolyl:
each R^{b55} is independently hydrogen, alkyl, carbocyclyl, heterocyclyl, aryl, heteroaryl, carbocyclylalkyl, heterocyclylalkyl, aralkyl or heteroarylalkyl; and
R^{b56} is alkyl.

Non-exhaustive examples of Formula I^{b5} include:

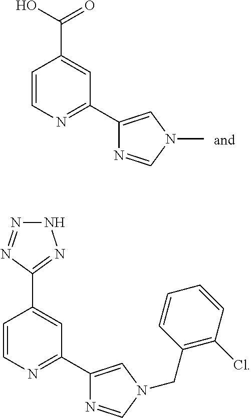 [SMILES: Cn1cnc(-c2cc(C(=O)O)ccn2)c1 ; Clc1ccccc1Cn1cnc(-c2cc(-c3nn[nH]n3)ccn2)c1]

In some embodiments of the invention, the KDM5 inhibitor is a compound of Formula I^{b5}:

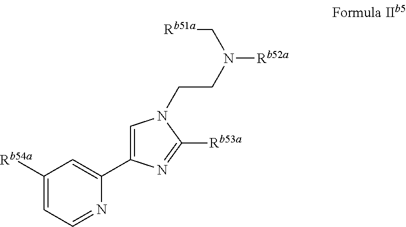

or a pharmaceutically acceptable salt thereof:
wherein:
R^{b51a} is carbocyclyl, heterocyclyl, aryl, or heteroaryl;
R^{b52a} is alkyl, carbocyclyl, heterocyclyl, aryl, heteroaryl, carbocyclylalkyl, heterocyclylalkyl, aralkyl, heteroarylalkyl, —CON(R^{b55a})₂, —CO₂R^{b55a}, —SON(R^{b55a})₂, or —SO₂R^{b55a}:
R^{b53a} is hydrogen, halogen, —OH, —NH₂, —NH(C₁₋₃ alkyl) or C₁₋₃ alkyl;
R^{b54a} is —CO₂H, —CO₂R^{b56a}, —C(O)N(H)CN, —C(O)N(H)OH or tetrazolyl;
each R^{b55a} is independently hydrogen, alkyl, carbocyclyl, heterocyclyl, aryl, heteroaryl, carbocyclylalkyl, heterocyclylalkyl, aralkyl or heteroarylalkyl; and
R^{b56a} is alkyl.

Non-exhaustive examples of Formula II^{b5} include:

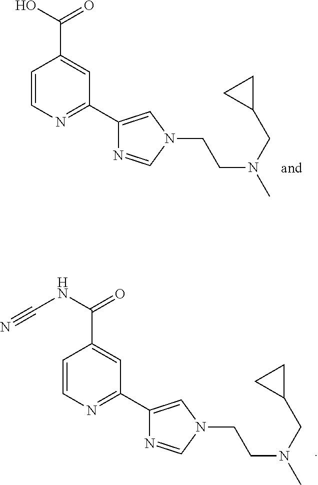 [SMILES: CN(CCn1cnc(-c2cc(C(=O)O)ccn2)c1)CC1CC1 ; "and" ; CN(CCn1cnc(-c2cc(C(=O)NC#N)ccn2)c1)CC1CC1 ; "."]

In some embodiments of the invention, the KDM5 inhibitor is a compound of Formula I^{b6}:

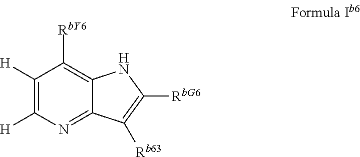

or a pharmaceutically acceptable salt thereof;
wherein:
R^{bY6} is —CO₂R^{b61}, —C(O)N(H)CN, —C(O)N(H)OH or tetrazolyl;
R^{b61} is hydrogen or alkyl:
R^{bG6} is R^{bX6}—R^{b62} or R^{bX61}-alkyl.
wherein
   R^{bX6} is a bond, alkylene, alkylene-O—, —C(O)—, —C(O)—NH—, —NH—, —NH—C(O)—, —O—, —S— or —SO₂—;
   R^{b62} is carbocyclyl, heterocyclyl, aryl or heteroaryl;
   R^{bX61} is a bond, —C(O)—, —C(O)—NH—, —NH—, —NH—C(O)—, —O—, —S— or —SO₂—; and
   R^{b63} is hydrogen, halogen or alkyl.

In further embodiments, Formula I^{b6} is represented by the structure of Formula II^{b6}:

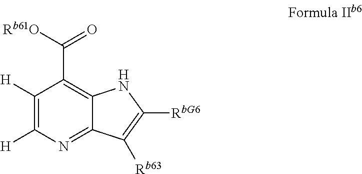

or a pharmaceutically acceptable salt thereof,
wherein,
R^{b61} is hydrogen or alkyl;
R^{bG6} is R^{bX6}—R^{b62} or R^{bX61}-alkyl,
wherein
   R^{bX6} is a bond, alkylene, alkylene-O—, —C(O)—, —C(O)—NH—, —NH—, —NH—C(O)—, —O—, —S— or —SO₂—;
   R^{b62} is selected from carbocyclyl, heterocyclyl, aryl or heteroaryl;
   R^{bX61} is a bond, —C(O)—, —C(O)—NH—, —NH—, —NH—C(O)—, —O—, —S— or —SO₂—; and
R^{b3} is hydrogen, halogen or alkyl.

Non-exhaustive examples of Formula I^{b6} include:

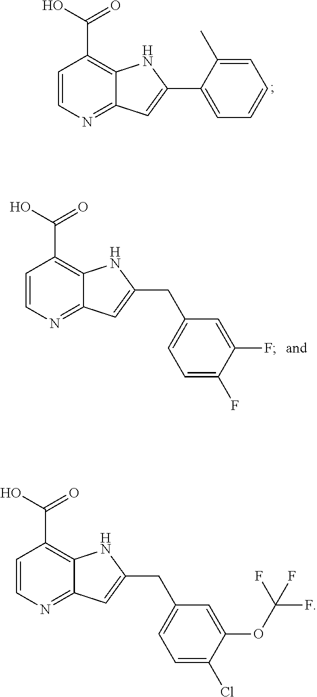 [SMILES: Cc1ccccc1-c1cc2nccc(C(=O)O)c2[nH]1 ; O=C(O)c1ccnc2cc(Cc3ccc(F)c(F)c3)[nH]c12 ; "and" ; O=C(O)c1ccnc2cc(Cc3ccc(Cl)c(OC(F)(F)F)c3)[nH]c12]

In some embodiments of the invention, the KDM5 inhibitor is a compound of Formula I^{c}:

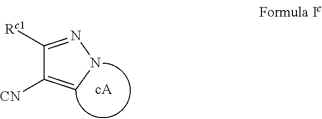

or a pharmaceutically acceptable salt thereof;
wherein:
R^{c1} is —R^{c}, halogen, —OR^{c}, —SR^{c}, —N(R^{c7})₂, —CN, —NO₂, —C(O)R^{c}, —CO₂R^{c}, —C(O)N(R^{c7})₂, —C(O)SR^{c}, —C(O)C(O)R^{c}, —C(O)CH₂C(O)R^{c}, —C(S)N(R^{c7})₂, —C(S)OR^{c}, —S(O)R^{c}, —SO₂R^{c}, —SO₂N(R^{c7})₂, —N(R^{c2})C(O)R^{c}, —N(R^{c7})C(O)N(R^{c7})₂, —N(R^{c7})SO₂R^{c}, —N(R^{c7})SO₂N(R^{c7})₂, —N(R^{c7})N(R^{c7})₂, —N(R^{c7})C(═N(R^{c7}))N(R^{c7})₂, —C═N(R^{c7})₂, —C═NOR^{c}, —C(═N(R C))N(R^{c7})₂, —OC(O)R^{c} or —OC(O)N(R^{c7})₂;
each R^{c} is independently hydrogen, optionally substituted C₁₋₆ aliphatic, optionally substituted phenyl, optionally substituted 3-7 membered carbocyclyl, optionally substituted 8-10 membered aryl, optionally substituted 5-10 membered heteroaryl, or optionally substituted 4-10 membered heterocyclyl;
each R^{c7} is independently —R^{c}, —C(O)R^{c}, —CO₂R^{c}; or
two R^{c7} on the same nitrogen are taken together with their intervening atoms to form a 4-7 membered heterocyclic ring having 1-2 heteroatoms independently selected from nitrogen, oxygen and sulfur;

Ring cA is

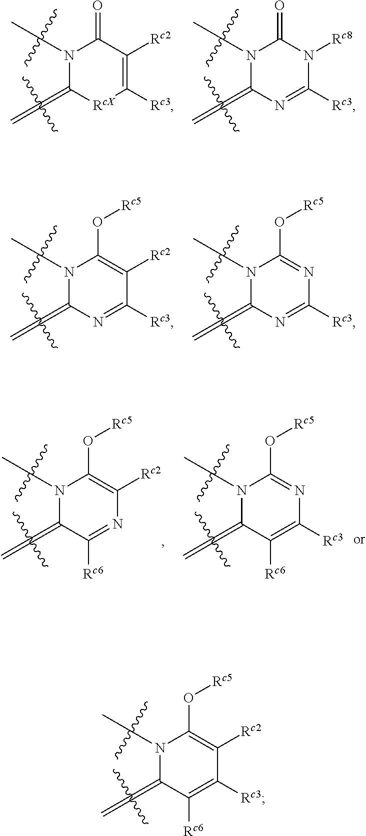

R^{c2} and R^{c3} are independently —R^{c}, halogen, —OR^{c}, —SR^{c}, —N(R^{c7})₂, —CN, —NO₂, —C(O)R^{c}, —CO₂R^{c}, —C(O)N(R^{c7})₂, —C(O)SR^{c}, —C(O)C(O)R^{c}, —C(O)CH₂C(O)R^{c}, —C(S)N(R^{c7})₂, —C(S)OR^{c}, —S(O)R^{c}, —SO₂R^{c}, —SO₂N(R^{c7})₂, —N(R^{c7})C(O)R^{c}, —N(R^{c7})C(O)N(R^{c7})₂, —N(R^{c7})SO₂R^{c}, —N(R^{c7})SO₂N(R^{c7})₂, —N(R^{c7})N(R^{c7})₂, —N(R^{c7})C(═N(R^{c7}))N(R^{c7})₂, —C═N(R^{c7})₂, —C═NOR^{c}, —C(═N(R^{c7})N(R^{c7})₂, —OC(O)R^{c} or —OC(O)N(R^{c7})₂; or
R^{c2} and R^{c3} are taken together with their intervening atoms to form an optionally substituted 5-7 membered partially unsaturated or aromatic fused ring having 0-4 heteroatoms independently selected from nitrogen, oxygen and sulfur;
R^{c8} is —R^{c}, —OR^{c}, —SR^{c}, —N(R^{c7})₂, —C(O)R^{c}, —CO₂R^{c}, —C(O)N(R^{c7})₂, —C(O)SR^{c}, —C(O)C(O)R^{c}, —C(O)CH₂C(O)R^{c}, —C(S)N(R^{c7})₂, —C(S)OR^{c}, —S(O)R^{c}, —SO₂R^{c}, —SO₂N(R^{c7})₂, —N(R^{c7})C(O)R^{c}, —N(R^{a7})C(O)N(R^{c7})₂, —N(R^{c7})SO₂R^{c}, —N(R^{c7})SO₂N(R^{c7})₂, —N(R^{c7})N(R^{c7})₂, —N(R^{c7})C(═N(R^{c7}))N(R^{c7})₂, —C═N(R^{c7})₂, —C═NOR^{c}, —C(═N(R^{c7}))N(R^{c7})₂, —OC(O)R^{c} or —OC(O)N(RCE)₂; or
R^{c8} and R^{c3} are taken together with their intervening atoms to form an optionally substituted 5-7 membered partially unsaturated or aromatic fused ring having 1-4 heteroatoms independently selected from nitrogen, oxygen and sulfur;
R^{cX} is —N(R^{c4})—, —O— or —S—;
R^{c4} is —R^{c}, —C(O)R^{c}, —CO₂R^{c} or —S(O)₂R^{c}; or:
R^{c4} and R^{c3} are taken together with their intervening atoms to form an optionally substituted 5-7 membered saturated, partially unsaturated, or aromatic fused ring having 1-4 heteroatoms independently selected from nitrogen, oxygen and sulfur:
R^{c6} is R^{c}, —C(O)R^{c}, —CO₂R^{c}, —C(O)N(R^{c7})₂, —C(O)C(O)R^{c}, or —C(O)CH₂C(O)R^{c}; or:
R^{c5} and R^{c2} are taken together with their intervening atoms to form an optionally substituted 5-7 membered partially unsaturated or aromatic fused ring having 1-4 heteroatoms independently selected from nitrogen, oxygen and sulfur; and
R^{c6} is —R^{c}, halogen, —OR^{c}, —SR^{c}, —N(R^{c7})₂, —CN, —NO₂, —C(O)R^{c}, —CO₂R^{c}, —C(O)N(R^{c7})₂, —C(O)SR^{c}, —C(O)C(O)R^{c}, —C(O)CH₂C(O)R^{c}, —C(S)N(R^{c7})₂, —C(S)OR^{c}, —S(O)R^{c}, —SO₂R^{c}, —SO₂N(R^{c7})₂, —N(R^{c7})C(O)R^{c}, —N(R^{c7})C(O)N(R^{c7})₂, —N(R^{c7})SO₂R^{c}, —N(R^{c7})SO₂N(R^{c7})₂, —N(R^{c7})N(R^{c7})₂, —N(R^{c7})C(═N(R^{c7}))N(R^{c7})₂, —C═N(R^{c7})₂, —C═NOR^{c}, —C(═N(R^{c7}))N(R^{c7})₂, —OC(O)R^{c} or —OC(O)N(R^{c7})₂; or:
R^{c6} and R^{c3} are taken together with their intervening atoms to form an optionally substituted 5-7 membered partially unsaturated or aromatic fused ring having 0-4 heteroatoms independently selected from nitrogen, oxygen and sulfur.

Non-exhaustive examples of Formula I^{c} include:

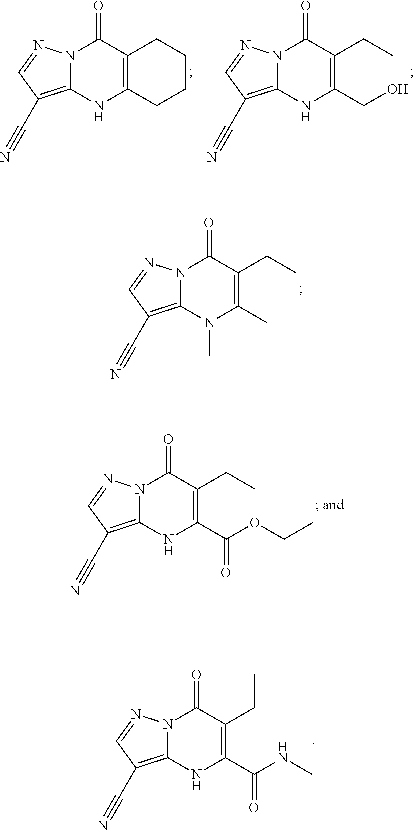

A further non-exhaustive example of Formula I^{c} includes:

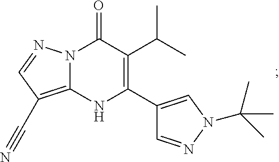 [SMILES: CC(C)c1c(-c2cnn(C(C)(C)C)c2)[nH]c2c(C#N)cnn2c1=O]

example 117 of United States Patent Publication no. US2016/0060267, published Mar. 3, 2016.

In some embodiments of the invention, the KDM5 inhibitor is a compound of Formula I^{c2}:

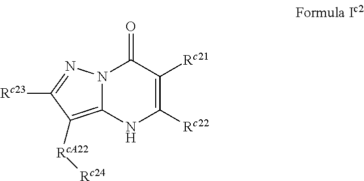

or a pharmaceutically acceptable salt thereof;
wherein:
R^{c21} and R^{c22} are each independently H, C₁₋₁₂alkyl, C₂₋₁₂alkenyl, C₂₋₁₂alkynyl, carbocyclyl, heterocyclyl, halo, —OR^{ca2}, —SR^{ca2}, —N(R^{ca2})₂, —CN, —NO₂, —C(O)R^{ca2}, —CO₂R^{ca2}, —C(O)N(R^{ca2})₂, —C(O)SR^{ca2}, —C(O)C(O)R^{ca2}, —C(O)CH₂C(O)R^{ca2}, —C(S)N(R^{ca2})₂, —C(S)OR^{ca2}, —S(O)R^{ca2}, —SO₂R^{ca2}, —SO₂N(R^{ca2})₂, —N(R^{ca2})C(O)R^{ca2}, —N(R^{ca2})C(O)N(R^{ca2})₂, —N(R^{ca2})SO₂R^{ca2}, —N(R^{ca2})SO₂N(R^{ca2})₂, —N(R^{ca2})N(R^{ca2})₂. —N(R^{ca2})C(═N(R^{ca2}))N(R^{ca2})₂, —C═NOR^{ca2}, —C(═N(R^{ca2}))N(R^{ca2})₂, —OC(O) R^{ca2}, or —OC(O)N(R^{ca2})₂;
   wherein each C₁₋₁₂alkyl, C₂₋₁₂alkenyl, C₂₋₁₂alkynyl, carbocyclyl, and heterocyclyl of R^{c21} and R^{c22} is independently optionally substituted with one or more groups R^{cx2}; and
   wherein R^{c21} and R^{c22} are not each H;
   or R^{c21} and R^{c22} taken together with the atoms to which they are attached form a 4, 5, 6, 7, or 8 membered carbocyclyl, which carbocyclyl is optionally substituted with one or more groups R^{cx2};
R^{c23} is H, C₁₋₆alkyl, trifluoromethyl, 3-6 membered carbocyclyl, 3-6 membered heterocyclyl, halo, —ORr, —SRr, —N(R^{cf2})₂, —CN, or —NO₂;
   wherein said alkyl, carbocyclyl and heterocyclyl are optionally substituted with one or more groups independently selected from oxo, halo, C₁₋₃alkoxy and C₁₋₃alkyl;
R^{c24} is H, C₁₋₁₂alkyl, C₂₋₁₂alkenyl, C₂₋₁₂alkynyl, carbocyclyl, heterocyclyl, halo, —OR^{cg2}, —SR^{cg2}, —N(R^{cg2})₂, —CN, —NO₂, —C(O)R^{cg2}, —CO₂R^{cg2}, —C(O)N(R^{cg2})₂, —C(O)SR^{cg2}, —C(O)C(O)R^{cg2}, —C(O)CH₂C(O)R^{cg2}, —C(S)N(R^{cg2})₂, —C(S)OR^{cg2}, —S(O)R^{cg2}, —SO₂R^{cg2}, —SO₂N(R^{cg2})₂, —N(R^{cg2})C(O)R^{cg2}, —N(R^{cg2})C(O)N(R^{cg2})₂, —N(R^{cg2})SO₂R^{cg2}, —N(R^{cg2})SO₂N(R^{cg2})₂, —N(R^{cg2})N(R^{cg2})₂, —N(R^{cg2})C(═N(R^{cg2}))N(R^{cg2})₂, —C(═N)N(R^{cg2})₂, —C═NO R^{cg2}, —C(═N(R^{cg2}))N(R^{cg2})₂, —OC(O)R^{cg2}, or —OC(O)N(R^{cg2})₂;
   wherein each C₁₋₁₂alkyl, C₂₋₁₂alkenyl, C₂₋₁₂alkynyl, carbocyclyl, and heterocyclyl of
      R^{c24} is optionally substituted with one or more groups R^{cx2};
R^{c25} is H, C₁₋₁₂alkyl, C₂₋₁₂alkenyl, C₂₋₁₂alkynyl, carbocyclyl, and heterocyclyl;
   wherein each C₁₋₁₂alkyl, C₂₋₁₂alkenyl, C₂₋₁₂alkynyl, carbocyclyl, and heterocyclyl is optionally substituted with one or more groups independently selected from oxo, C₁₋₁₂alkyl, C₁₋₁₂haloalkyl, carbocyclyl, heterocyclyl, halo, —CN, —NO2, —N^{cm2}R^{cm2}, —OR^{cm2}, —C(═O)OR^{cm2}, and —OC(═O)R^{cm2};
   or R^{c25} and R^{c22} taken together with the atoms to which they are attached form a heterocyclyl;
each R^{ca2} is independently selected from H, C₁₋₆alkyl, C₂₋₆alkenyl, C₂₋₆alkynyl, carbocyclyl, and heterocyclyl;
   wherein each C₁₋₆alkyl, C₂₋₆alkenyl, C₂₋₆alkynyl, carbocyclyl, and heterocyclyl is optionally substituted with one or more groups R^{cx2};
each R^{cf2} is independently selected from H, C₁₋₃alkyl, trifluoromethyl, 3-6 membered carbocyclyl, and 3-6 membered heterocyclyl;
   or two R^{cf2} groups together with the nitrogen to which they are attached form a 3-6 membered heterocycle;
each R^{cg2} is independently selected from H, C₁₋₆alkyl, C₂₋₆alkenyl, C₂₋₆alkynyl, C₃₋₈carbocyclyl, and heterocyclyl, wherein each C₁₋₆alkyl, C₂₋₆alkenyl, C₂₋₆alkynyl, C₃₋₈carbocyclyl, and heterocyclyl is optionally substituted with one or more groups R^{cx2};
   or two R^{cg2} groups together with the nitrogen to which they are attached form a 3-6 membered heterocycle;
each R^{cm2} is independently selected from H, C₁₋₆alkyl, C₂₋₆alkenyl, C₂₋₆alkynyl, C₁₋₆haloalkyl, carbocyclyl, C₁₋₆alkanoyl, phenyl, and benzyl,
   wherein any C₁₋₆alkyl, C₂₋₆alkenyl, C₂₋₆alkynyl, C₁₋₆haloalkyl, carbocyclyl, C₁₋₆alkanoyl, phenyl, or benzyl is optionally substituted with one or more groups independently selected from halo, —CN, —NO2, —NR^{cy2}R^{cz2}, and —OR^{cw2};
   or two R^{cm2} groups together with the nitrogen to which they are attached form a 3-6 membered heterocycle;
R^{cA22} is a monocyclic or bicyclic heteroaryl ring that is substituted with R^{c24} and that is also optionally substituted with one or more groups independently selected from halo, nitro, cyano, C₁₋₆alkyl, C₂₋₆alkenyl, C₂₋₆alkynyl, C₁₋₆haloalkyl, —OR^{ct2}, —C(O)R^{ct2}, —CO₂R^{ct2}, —OC(O)R^{ct2}, —N(R^{ct2})₂, and carbocyclyl;
each R^{ct2} is independently selected from H, C₁₋₆alkyl, C₂₋₆alkenyl, C₂₋₆alkynyl, C₃₋₈carbocyclyl, and heterocyclyl;
   wherein each C₁₋₆alkyl, C₂₋₆alkenyl, C₂₋₆alkynyl, C₃₋₈carbocyclyl, and heterocyclyl is optionally substituted with one or more groups R^{cx2};
   or two R^{ct2} groups together with the nitrogen to which they are attached form a 3-6 membered heterocycle;
each R^{cv2} is independently hydrogen, C₁₋₆alkyl, C₂₋₆alkenyl, C₂₋₆alkynyl, carbocyclyl, and heterocyclyl,
   wherein each C₁₋₆alkyl, C₂₋₆alkenyl, C₂₋₆alkynyl, carbocyclyl, and heterocyclyl is optionally substituted with one or more groups independently selected from oxo, halo, amino, hydroxyl, and C₁₋₆alkyl that is optionally substituted with one or more groups independently selected from oxo and halo;
   or two R^{cv2} are taken together with the nitrogen to which they are attached to form a heterocyclyl that is optionally substituted with one or more groups independently selected from oxo, halo and C₁₋₃alkyl that is optionally substituted with one or more groups independently selected from oxo and halo;
each R^{cw2} is independently selected from H, C₁₋₄alkyl, C₁₋₄alkanoyl, phenyl, benzyl, and phenethyl;
each R^{cx2} is independently selected from oxo, C₁₋₆alkyl, C₂₋₆alkenyl, C₂₋₆alkynyl, C₁₋₆haloalkyl, carbocyclyl, —F, —Cl, —Br, —I, —NO₂, —N(R^{cv2})₂, —CN, —C(O)—N(R^{c7})₂, —S(O)—N(R^{cv2})₂, —S(O)₂—N(R^{cv2})₂, —O—R^{cv2}, —S—R^{cv2}, —O—C(O)—R^{cv2}, —O—C(O)—O—R^{cv2}, —C(O)—R^{cv2}, —C(O)—O—R^{cv2}, —S(O)—R^{cv2}, —S(O)₂—R^{cv2}, —O—C(O)—N(R^{cv2})₂, —N(R^{cv2})—C(O)—OR^{cv2}, —N(R^{cv2})—C(O)—N(R^{cv2})₂, —S(O)₂—N(R^{cv2})₂, —N(R^{cv2})—C(O)—R^{cv2}, —N(R^{cv2})S(O)—R^{cv2}, —N(R^{cv2})—S(O)₂—R^{cv2}, —N(R^{cv2})—S(O)—N(R^{cv2})₂, and —N(R^{cv2})—S(O)₂—N(R^{cv2})₂,
   wherein any C₁₋₆alkyl, C₂₋₆alkenyl, C₂₋₆alkynyl, C₁₋₆haloalkyl and carbocyclyl is optionally substituted with one or more groups independently selected from oxo, halo, —NO₂, —N(R^{cv2})₂, —CN, —C(O)—N(R^{cv2})₂, S(O)—N(R^{cv2}), —S(O)₂—N(R^{cv2})₂, —O—R^{cv2}, —S—R^{cv2}, —O—C(O)—R^{cv2}, —C(O)—R^{cv2}, —C(O)—O—R^{cv2}, —S(O)—R^{cv2}, —S(O)₂—R^{cv2}, —C(O)—N(R^{cv2})₂, —S(O)₂—N(R^{cv2})₂, —N(R^{cv2})—C(O)—R^{cv2}, —N(R^{cv2})—S(O)—R^{cv2}—N(R^{cv2})—S(O)₂—R^{cv2} and C₁₋₆alkyl that is optionally substituted with one or more groups independently selected from oxo and halo; and
each R^{cY2} and R^{cz2} is independently selected from H, C₁₋₄alkyl, C₁₋₄alkanoyl, C₁₋₄alkoxycarbonyl, phenyl, benzyl, and phenethyl, or R^{cY2} and R^{cz2} together with the nitrogen to which they are attached form a heterocyclyl.

Non-exhaustive examples of Formula I^{c2} include:

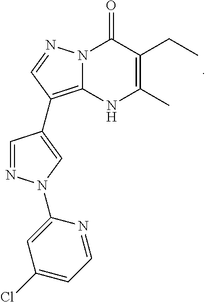 [SMILES: CCc1c(C)[nH]c2c(-c3cnn(-c4cc(Cl)ccn4)c3)cnn2c1=O]

Further embodiments of the KDM5 inhibitor may be selected from

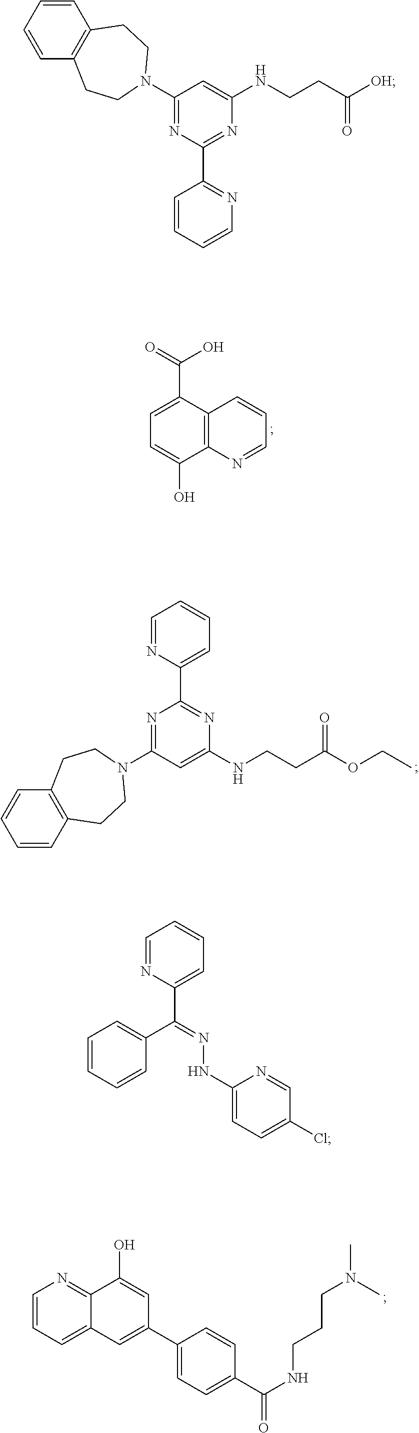 [SMILES: O=C(O)CCNc1cc(N2CCc3ccccc3CC2)nc(-c2ccccn2)n1 ; O=C(O)c1ccc(O)c2ncccc12 ; CCOC(=O)CCNc1cc(N2CCc3ccccc3CC2)nc(-c2ccccn2)n1 ; Clc1ccc(N/N=C(\c2ccccc2)c2ccccn2)nc1 ; CN(C)CCCNC(=O)c1ccc(-c2cc(O)c3ncccc3c2)cc1]

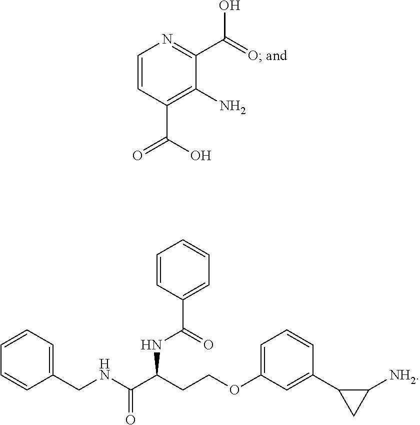

Unless otherwise specified, the phrase “one or more” in the above formulae may include 1, 2 or 3, for example 1 or 2.

In one embodiment, the KDM5 inhibitor is:

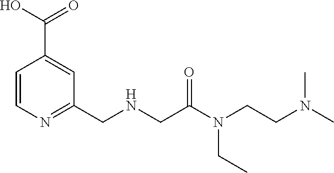 [SMILES: CCN(CCN(C)C)C(=O)CNCc1cc(C(=O)O)ccn1]

or a pharmaceutically acceptable salt and/or prodrug thereof.

In one embodiment, the KDM5 inhibitor is:

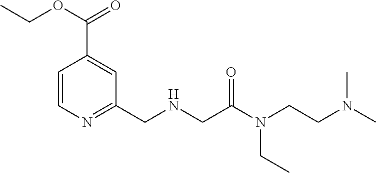 [SMILES: CCOC(=O)c1ccnc(CNCC(=O)N(CC)CCN(C)C)c1]

or a pharmaceutically acceptable salt thereof.

In one embodiment, the KDM5 inhibitor is:

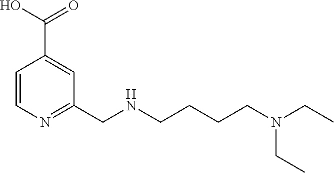 [SMILES: CCN(CC)CCCCNCc1cc(C(=O)O)ccn1]

or a pharmaceutically acceptable salt thereof.

In one embodiment, the KDM5 inhibitor is:

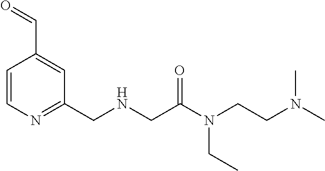 [SMILES: CCN(CCN(C)C)C(=O)CNCc1cc(C=O)ccn1]

or a pharmaceutically acceptable salt thereof.

In one embodiment, the KDM5 inhibitor is:

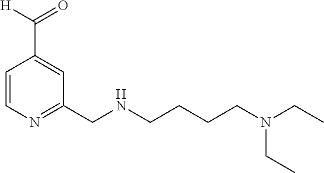 [SMILES: CCN(CC)CCCCNCc1cc(C=O)ccn1]

or a pharmaceutically acceptable salt thereof.

In one embodiment, the compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} for use in a method of treating HBV. In one embodiment, a compound of Formula I^{a} for use in a method of treating HBV. In one embodiment, a compound of Formula I^{a1} for use in a method for treating HBV.

In one embodiment, the compound:

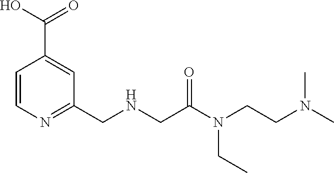 [SMILES: CCN(CCN(C)C)C(=O)CNCc1cc(C(=O)O)ccn1]

or a pharmaceutically acceptable salt and/or prodrug thereof, for use in a method of treating HBV.
In one embodiment, the compound:

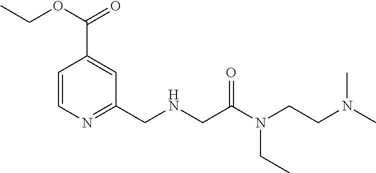 [SMILES: CCOC(=O)c1ccnc(CNCC(=O)N(CC)CCN(C)C)c1]

or a pharmaceutically acceptable salt thereof, for use in a method of treating HBV.

In one embodiment, the compound:

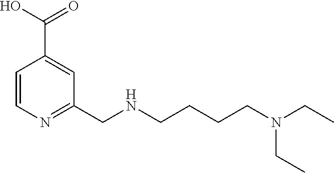 [SMILES: CCN(CC)CCCCNCc1cc(C(=O)O)ccn1]

or a pharmaceutically acceptable salt thereof, for use in a method of treating HBV.

In one embodiment, the compound:

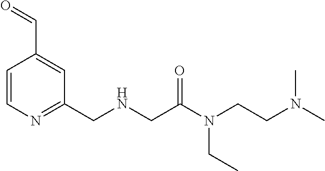 [SMILES: CCN(CCN(C)C)C(=O)CNCc1cc(C=O)ccn1]

or a pharmaceutically acceptable salt thereof, for use in a method of treating HBV.

In one embodiment, the compound:

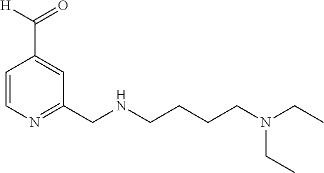 [SMILES: CCN(CC)CCCCNCc1cc(C=O)ccn1]

or a pharmaceutically acceptable salt thereof, for use in a method of treating HBV.

In one embodiment, use of a compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, I^{c}, and I^{c2} or a pharmaceutically acceptable salt thereof, in the manufacture of a medicament for treating HBV. for use in a method of treating HBV. In one embodiment, use of a compound of Formulae I^{a}, or a pharmaceutically acceptable salt thereof, in the manufacture of a medicament for treating HBV. In one embodiment, use of a compound of Formulae I^{a1} or a pharmaceutically acceptable salt thereof, in the manufacture of a medicament for treating HBV.

In one embodiment, use of the compound

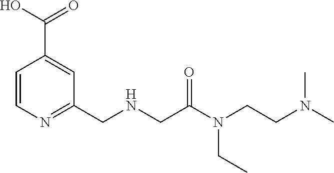 [SMILES: CCN(CCN(C)C)C(=O)CNCc1cc(C(=O)O)ccn1]

or a pharmaceutically acceptable salt and/or prodrug thereof, for treating HBV.

In one embodiment, use of the compound

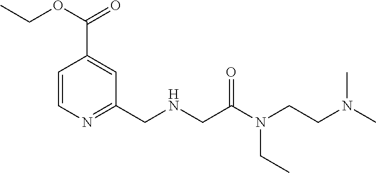 [SMILES: CCOC(=O)c1ccnc(CNCC(=O)N(CC)CCN(C)C)c1]

or a pharmaceutically acceptable salt and/or prodrug thereof, for treating HBV. In one embodiment, use of the compound

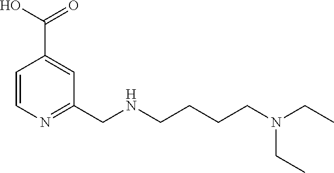 [SMILES: CCN(CC)CCCCNCc1cc(C(=O)O)ccn1]

or a pharmaceutically acceptable salt and/or prodrug thereof, for treating HBV.

In one embodiment, use of the compound

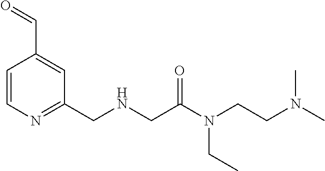 [SMILES: CCN(CCN(C)C)C(=O)CNCc1cc(C=O)ccn1]

or a pharmaceutically acceptable salt and/or prodrug thereof, for treating HBV.

In one embodiment, use of the compound

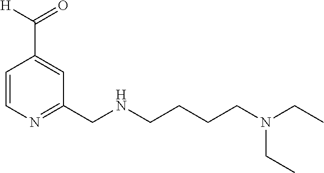 [SMILES: CCN(CC)CCCCNCc1cc(C=O)ccn1]

or a pharmaceutically acceptable salt and/or prodrug thereof, for treating HBV.

The compounds of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Localized administration is a preferred embodiment. An embodiment includes administration once a day (QD). Another embodiment includes administration twice a day (BID).

The compounds of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Localized administration is a preferred embodiment. An embodiment includes administration once a day (QD). Another embodiment includes administration twice a day (BID).

In one aspect, the compounds described herein may be administered orally. Oral administration may be via, for example, capsule or enteric coated tablets. In making the pharmaceutical compositions that include at least one compound of Formulae I^{a}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2}, or a pharmaceutically acceptable salt, is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In one aspect, the compounds described herein may be administered orally. Oral administration may be via, for example, capsule or enteric coated tablets. In making the pharmaceutical compositions that include at least one compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2}, or a pharmaceutically acceptable salt, is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinvlpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The methods that include at least one compound of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2} or a pharmaceutically acceptable salt, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The methods that include at least one compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2} or a pharmaceutically acceptable salt, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525, 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See. e.g., U.S. Pat. Nos. 5,023,252, 4,992,445 and 5,001,139. Such patches may be constructed for continuous, pulsatile, or on demand delivery of pharmaceutical agents

The compositions may, in some embodiments, be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, for oral administration, each dosage unit contains from about 10 mg to about 1000 mg of a compound described herein, for example from about 50 mg to about 500 mg, for example about 50 mg, about 75 mg, about 100 mg, about 150 mg, about 200 mg, about 250 mg, or about 300 mg. In other embodiments, for parenteral administration, each dosage unit contains from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, and the severity of the subject's symptoms.

The compositions may, in some embodiments, be formulated in pulse dosing regimens.

The compositions may, in some embodiments, be formulated where a KDM5 inhibitor is administered once daily for one day and then not administered for a following one day.

The compositions may, in some embodiments, be formulated where a KDM5 inhibitor is administered once daily for seven days and then not administered for a following seven days.

In certain embodiments, dosage levels may be from 0.1 mg to 100 mg per kilogram of body weight per day, for example from about 1 mg to about 50 mg per kilogram, for example from about 5 mg to about 30 mg per kilogram. Such dosage levels may, in certain instances, be useful in the treatment of the above-indicated conditions. In other embodiments, dosage levels may be from about 10 mg to about 2000 mg per subject per day. The amount of active ingredient that may be combined with the vehicle to produce a single dosage form will vary depending upon the host treated and the particular mode of administration. Dosage unit forms may contain from 1 mg to 500 mg of an active ingredient.

In some embodiments, dosage unit forms contain from 1 mg to 100 mg of an active ingredient. In some embodiments, dosage unit forms contain from 1 mg to 10 mg of an active ingredient. In some embodiments, dosage unit forms contain from 50 mg to 100 mg of an active ingredient.

Frequency of dosage may also vary depending on the compound used and the particular disease or condition treated. In some embodiments, for example, for the treatment of an autoimmune and/or inflammatory disease, a dosage regimen of 4 times daily or less is used. In some embodiments, a dosage regimen of 1 or 2 or 3 times daily is used. It will be understood, however, that the specific dose level for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For preparing solid compositions such as tablets, the principal active ingredient may be mixed with a pharmaceutical excipient to form a solid preformulation composition containing a homogeneous mixture of a compound of Formulae I^{a}, I^{a2}, I^{b2}, II^{b2} I^{b3}, I^{b3a}, I^{b3b}, II^{b3}, I^{b4}, I^{b5}, I^{b6}, II^{b6}, I^{c} and I^{c2}, or a pharmaceutically acceptable salt, thereof. When referring to these preformulation compositions as homogeneous, the active ingredient may be dispersed evenly throughout the composition so that the composition may be readily subdivided into equally effective unit dosage forms such as tablets, pills and capsules.

For preparing solid compositions such as tablets, the principal active ingredient may be mixed with a pharmaceutical excipient to form a solid preformulation composition containing a homogeneous mixture of a compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2}, or a pharmaceutically acceptable salt, thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

RNA sequences SEQ ID NOs 1, 2, 3, 4, 5, 6, 7, and 8 may be administered in either single or multiple doses by any of the accepted modes of administration of agents having similar utilities, for example as described in those patents and patent applications incorporated by reference, including localized (via liver, lung, brain spinal cord, or isolated tumor), topical (via eye, skin, vagina, or rectum), or systemic (via liver, heart, kidney, or metastasized tumor) delivery systems. (Whitehead K A, et al. Nature Reviews Drug Discovery 8, 129-138 (February 2009)|doi:10.1038/nrd2742; Vicentini F T, et al. Pharm Res 2013; 30:915-931. doi 10.1007/s11095-0130971-1). Localized administration is a preferred embodiment.

### Combination Therapy

In certain embodiments, a method for treating or preventing an HBV infection in a human having or at risk of having the infection is provided, comprising administering to the human a therapeutically effective amount of a compound disclosed herein, or a pharmaceutically acceptable salt thereof, in combination with a therapeutically effective amount of one or more (e.g., one, two, three, four, one or two, or one to three, or one to four) additional therapeutic agents. In one embodiment, a method for treating an HBV infection in a human having or at risk of having the infection is provided, comprising administering to the human a therapeutically effective amount of a compound disclosed herein, or a pharmaceutically acceptable salt thereof, in combination with a therapeutically effective amount of one or more (e.g., one, two, three, four, one or two, or one to three, or one to four) additional therapeutic agents.

In certain embodiments, the present disclosure provides a method for treating an HBV infection, comprising administering to a patient in need thereof a therapeutically effective amount of a compound disclosed herein, or a pharmaceutically acceptable salt thereof, in combination with a therapeutically effective amount of one or more additional therapeutic agents which are suitable for treating an HBV infection.

In certain embodiments, a compound as disclosed herein (e.g., any compound of Formula I^{a}, I^{a2}, I^{b}, I^{b2}, I^{b3}, I^{b3a}, I^{b3b}, II^{b3}, I^{b4}, I^{b5}, I^{b6}, II^{b6} or I^{c}) may be combined with one or more additional therapeutic agents in any dosage amount of the compound of Formula I^{a}, I^{a2} I^{b}, I^{b2}, II^{b2}, I^{b3}, I^{b3a}, I^{b3b}, II^{b3}, I^{b4}, I^{b5}, I^{b6}, II^{b6} or I^{c} (e.g., from 10 mg to 1000 mg of compound).

In certain embodiments, a compound as disclosed herein (e.g., any compound of Formula I^{a}, I^{a1}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} or I^{c2}) may be combined with one or more additional therapeutic agents in any dosage amount of the compound of Formula I^{a}, I^{a1}, I^{a2}, I^{b}, I^{b2}, I^{b3}, I^{b3a}, I^{b3b}, II^{b3}, I^{b4}, I^{b5}, I^{b6}, II^{b6}, I^{c} or I^{c2} (e.g., from 10 mg to 1000 mg of compound).

In one embodiment, pharmaceutical compositions comprising a compound disclosed herein, or a pharmaceutically acceptable salt thereof, in combination with one or more (e.g., one, two, three, four, one or two, or one to three, or one to four) additional therapeutic agents, and a pharmaceutically acceptable carrier, diluent or excipient are provided.

In one embodiment, kits comprising a compound disclosed herein, or a pharmaceutically acceptable salt thereof, in combination with one or more (e.g., one, two, three, four, one or two, or one to three, or one to four) additional therapeutic agents are provided.

In the above embodiments, the additional therapeutic agent may be an anti-HBV agent. For example, in some embodiments, the additional therapeutic agent is selected from the group consisting of HBV combination drugs, HBV DNA polymerase inhibitors, immunomodulators, toll-like receptor modulators (modulators of tlr1, tlr2, tlr3, tlr4, tlr5, tlr6, tlr7, tlr8, tlr9, tlr10, tlr11, tlr12 and tlr13), interferon alpha receptor ligands, hyaluronidase inhibitors, recombinant IL-7, hepatitis B surface antigen (HBsAg) inhibitors, compounds targeting hepatitis B core antigen (HbcAg), cyclophilin inhibitors, HBV therapeutic vaccines, HBV prophylactic vaccines, HBV viral entry inhibitors, NTCP (Na+-taurocholate cotransporting polypeptide) inhibitors, antisense oligonucleotide targeting viral mRNA, short interfering RNAs (siRNA), miRNA gene therapy agents, endonuclease modulators, inhibitors of ribonucleotide reductase, hepatitis B virus E antigen inhibitors, recombinant scavenger receptor A (SRA) proteins, Src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators. Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambda, recombinant thymosin alpha-1, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), Hepatitis B virus replication inhibitors compounds such as those disclosed in US20100143301 (Gilead Sciences), US20110098248 (Gilead Sciences), US20090047249 (Gilead Sciences), U.S. Pat. No. 8,722,054 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (VentirxPharma), US20140275167 (Novira therapeutics), US20130251673 (Novira therapeutics), U.S. Pat. No. 8,513,184 (Gilead Sciences), US20140030221 (Gilead Sciences), US20130344030 (Gilead Sciences), US20130344029 (Gilead Sciences), US20140343032 (Roche), WO2014037480 (Roche), US20130267517 (Roche), WO2014131847 (Janssen), WO2014033176 (Janssen), WO2014033170 (Janssen), WO2014033167 (Janssen), U S20140330015 (Ono pharmaceutical), US20130079327 (Ono pharmaceutical), US20130217880 (Ono pharmaceutical), and other drugs for treating HBV, and combinations thereof.

In certain embodiments, the additional therapeutic is selected from the group consisting of HBV combination drugs, HBV DNA polymerase inhibitors, toll-like receptor 7 modulators, toll-like receptor 8 modulators, Toll-like receptor 7 and 8 modulators, Toll-like receptor 3 modulators, interferon alpha receptor ligands, HBsAg inhibitors, compounds targeting HbcAg, cyclophilin inhibitors, HBV therapeutic vaccines, HBV prophylactic vaccines, HBV viral entry inhibitors, NTCP inhibitors, antisense oligonucleotide targeting viral mRNA, short interfering RNAs (siRNA), hepatitis B virus E antigen inhibitors, HBx inhibitors, cccDNA inhibitors, HBV antibodies including HBV antibodies targeting the surface antigens of the hepatitis B virus, thymosin agonists, cytokines, nucleoprotein inhibitors (HBV core or capsid protein inhibitors), stimulators of retinoic acid-inducible gene 1, stimulators of NOD2, stimulators of NOD1, recombinant thymosin alpha-1, BTK inhibitors, and hepatitis B virus replication inhibitors, and combinations thereof.

In certain embodiments a compound of Formula I^{a}, I^{a2}, I^{b}, I^{b2}, II^{b2}, I^{b3}, I^{b3a}, I^{b3b}, II^{b3}, I^{b4}, I^{b5}, I^{b6}, II^{b6} or I^{c} is formulated as a tablet, which may optionally contain one or more other compounds useful for treating HBV. In certain embodiments, the tablet can contain another active ingredient for treating HBV, such as HBV DNA polymerase inhibitors, immunomodulators, toll-like receptor modulators (modulators of tlr1, tlr2, tlr3, tlr4, tlr5, tlr6, tlr7, tlr8, tlr9, tlr10, tlr11, tlr12 and tlr13), modulators of tlr7, modulators of tlr8, modulators of tlr7 and tlr8, interferon alpha receptor ligands, hyaluronidase inhibitors, hepatitis B surface antigen (HBsAg) inhibitors, compounds targeting hepatitis B core antigen (HbcAg), cyclophilin inhibitors, HBV viral entry inhibitors, NTCP (Na+-taurocholate cotransporting polypeptide) inhibitors, endonuclease modulators, inhibitors of ribonucleotide reductase, hepatitis B virus E antigen inhibitors, Src kinase inhibitors, HBx inhibitors, cccDNA inhibitors, CCR2 chemokine antagonists, thymosin agonists, nucleoprotein inhibitors (HBV core or capsid protein inhibitors), stimulators of retinoic acid-inducible gene 1, stimulators of NOD2, stimulators of NOD1. Arginase-1 inhibitors, STING agonists. PI3K inhibitors, lymphotoxin beta receptor activators. Natural Killer Cell Receptor 2B4 inhibitors. Lymphocyte-activation gene 3 inhibitors, CD160 inhibitors, cytotoxic T-lymphocyte-associated protein 4 inhibitors. CD137 inhibitors. Killer cell lectin-like receptor subfamily G member 1 inhibitors, TIM-3 inhibitors, B- and T-lymphocyte attenuator inhibitors. CD305 inhibitors, PD-1 inhibitors, PD-L1 inhibitors, BTK inhibitors, modulators of TIGIT, modulators of CD47, modulators of SIRP alpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, and Hepatitis B virus replication inhibitors, and combinations thereof.

In certain embodiments a compound of Formula I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} or I^{c2} is formulated as a tablet, which may optionally contain one or more other compounds useful for treating HBV. In certain embodiments, the tablet can contain another active ingredient for treating HBV, such as HBV DNA polymerase inhibitors, immunomodulators, toll-like receptor modulators (modulators of tlr1, tlr2, tlr3, tlr4, tlr5, tlr6, tlr7, tlr8, tlr9, tlr10, tlr11, tlr12 and tlr13), modulators of tlr7, modulators of tlr8, modulators of tlr7 and tlr8, interferon alpha receptor ligands, hyaluronidase inhibitors, hepatitis B surface antigen (HBsAg) inhibitors, compounds targeting hepatitis B core antigen (HbcAg), cyclophilin inhibitors, HBV viral entry inhibitors, NTCP (Na+-taurocholate cotransporting polypeptide) inhibitors, endonuclease modulators, inhibitors of ribonucleotide reductase, hepatitis B virus E antigen inhibitors, Src kinase inhibitors, HBx inhibitors, cccDNA inhibitors, CCR2 chemokine antagonists, thymosin agonists, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators. Natural Killer Cell Receptor 2B4 inhibitors. Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors. CD305 inhibitors, PD-1 inhibitors, PD-L1 inhibitors, BTK inhibitors, modulators of TIGIT, modulators of CD47, modulators of SIRP alpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, and Hepatitis B virus replication inhibitors, and combinations thereof.

In certain embodiments, such tablets are suitable for once daily dosing.

In certain embodiments, the additional therapeutic agent is selected from one or more of:

(1) Combination drugs selected from the group consisting of tenofovir disoproxil fumarate+emtricitabine (Truvada®) adefovir+clevudine, ABX-203+lamivudine+PEG-IFNalpha, ABX-203+adefovir+PEG-IFNalpha and GBV-015;

(2) HBV DNA polymerase inhibitors selected from the group consisting of besifovir, entecavir (Baracludek), adefovir (Hepsera®), tenofovir disoproxil fumarate (Vireadf), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, telbivudine (Tyzeka®), pradefovir, Clevudine, emtricitabine (Emtriva®), ribavirin, lamivudine (Epivir-HBV®), phosphazide, famciclovir, SNC-019754, FMCA, fusolin, AGX-1009 and metacavir;

(3) Immunomodulators selected from the group consisting of rintatolimod, imidol hydrochloride, ingaron, dermaVir, plaquenil (hydroxychloroquine), proleukin, hydroxyurea, mycophenolate mofetil (MPA) and its ester derivative mycophenolate mofetil (MMF), WF-10, ribavirin, IL-12, polymer polyethyleneimine (PEI), Gepon, VGV-1, MOR-22, BMS-936559 and IR-103;

(4) Toll-like receptor 7 modulators selected from the group consisting of GS-9620, GSK-2245035, imiquimod, resiquimod, DSR-6434, DSP-3025, IMO-4200, MCT-465, 3M-051, SB-9922, 3M-052, Limtop, TMX-30X, TMX-202 RG-7863 and RG-7795;

(5) Toll-like receptor 8 modulators selected from the group consisting of motolimod, resiquimod, 3M-051, 3M-052. MCT-465, IMO-4200, VTX-763, VTX-1463;

(6) Toll-like receptor 3 modulators selected from the group consisting of rintatolimod, poly-ICLC, MCT-465, MCT-475, Riboxxon, Riboxxim and ND-1.1;

(7) Interferon alpha receptor ligands selected from the group consisting of interferon alpha-2b (Intron A®), pegylated interferon alpha-2a (Pegasys®), interferon alpha 1b (Hapgen®), Veldona, Infradure, Roferon-A, YPEG-interferon alfa-2a (YPEG-rhIFNalpha-2a), P-1101, Algeron, Alfarona, Ingaron (interferon gamma), rSIFN-co (recombinant super compound interferon), Ypeginterferon alfa-2b (YPEG-rhlFNalpha-2b), MOR-22, peginterferon alfa-2b (PEG-Intron®), Bioferon, Novaferon, Inmutag (IFN), Multiferon®, interferon alfa-n1 (Humoferon®), interferon beta-1a (Avonext), Shaferon, interferon alfa-2b (AXXO), Alfaferone, interferon alfa-2b (BioGeneric Pharma), interferon-alpha 2 (CJ), Laferonum, VIPEG, BLAUFERON-B, BLAUFERON-A, Intermax Alpha, Realdiron, Lanstion, Pegaferon, PDferon-B PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b and Interapo (Interapa);

(8) Hyaluronidase inhibitors selected from the group consisting of astodrimer;

(9) Modulators of IL-10;

(10) HBsAg inhibitors selected from the group consisting of HBF-0259, PBHBV-001, PBHBV-2-15, PBHBV-2-1, REP 9AC, REP-9C and REP 9AC′;

(11) Toll like receptor 9 modulators selected from CYT003;

(12) Cyclophilin inhibitors selected from the group consisting of OCB-030, SCY-635 and NVP-018;

(13) HBV Prophylactic vaccines selected from the group consisting of Hexaxim, Heplisav, Mosquirix, DTwP-HBV vaccine, Bio-Hep-B, D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, Engerix B®, recombinant hepatitis B vaccine (intramuscular, Kangtai Biological Products), recombinant hepatitis B vaccine (Hansenual polymorpha yeast, intramuscular, Hualan Biological Engineering), Bimmugen, Euforavac, Eutravac, anrix-DTaP-IPV-Hep B, Infanrix-DTaP-IPV-Hep B-Hib, Pentabio Vaksin DTP-HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan6, rhHBsAG vaccine, and DTaP-rHB-Hib vaccine;

(14) HBV Therapeutic vaccines selected from the group consisting of HBsAG-HBIG complex, Bio-Hep-B, NASVAC, abi-HB (intravenous), ABX-203, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, TG-1050, NU-500, HBVax, im/TriGrid/antigen vaccine, Mega-CD40L-adjuvanted vaccine, HepB-v, NO-1800, recombinant VLP-based therapeutic vaccine (HBV infection, VLP Biotech), AdTG-17909, AdTG-17910 AdTG-18202, ChronVac-B, and Lm HBV;

(15) HBV viral entry inhibitor selected from the group consisting of Myrcludex B;

(16) Antisense oligonucleotide targeting viral mRNA selected from the group consisting of ISIS-HBVRx;

(17) short interfering RNAs (siRNA) selected from the group consisting of TKM-HBV (TKM-HepB), ALN-HBV, SR-008, ddRNAi and ARC-520;

(18) Endonuclease modulators selected from the group consisting of PGN-514;

(19) Inhibitors of ribonucleotide reductase selected from the group consisting of Trimidox;

(20) Hepatitis B virus E antigen inhibitors selected from the group consisting of wogonin;

(21) HBV antibodies targeting the surface antigens of the hepatitis B virus selected from the group consisting of GC-1102, XTL-17, XTL-19, XTL-001, KN-003 and fully human monoclonal antibody therapy (hepatitis B virus infection, Humabs BioMed);

(22) HBV antibodies including monoclonal antibodies and polyclonal antibodies selected from the group consisting of Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, hepatitis B immunoglobulin (intravenous, pH4, HBV infection, Shanghai RAAS Blood Products) and Fovepta (BT-088);

(23) CCR2 chemokine antagonists selected from the group consisting of propagermanium;

(24) Thymosin agonists selected from the group consisting of Thymalfasin;

(25) Cytokines selected from the group consisting of recombinant IL-7, CYT-107, interleukin-2 (IL-2, Immunex); recombinant human interleukin-2 (Shenzhen Neptunus) and celmoleukin;

(26) Nucleoprotein inhibitors (HBV core or capsid protein inhibitors) selected from the group consisting of NVR-1221, NVR-3778, BAY 41-4109, morphothiadine mesilate and DVR-23;

(27) Stimulators of retinoic acid-inducible gene 1 selected from the group consisting of SB-9200, SB-40, SB-44, ORI-7246, ORI-9350, ORI-7537. ORI-9020, ORI-9198 and ORI-7170;

(28) Stimulators of NOD2 selected from the group consisting of SB-9200;

(29) Recombinant thymosin alpha-1 selected from the group consisting of NL-004 and PEGylated thymosin alpha 1;

(30) Hepatitis B virus replication inhibitors selected from the group consisting of isothiafludine, IQP-HBV, RM-5038 and Xingantie;

(31) PI3K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;

(32) cccDNA inhibitors selected from the group consisting of BSBI-25;

(33) PD-L1 inhibitors selected from the group consisting of MEDI-0680, RG-7446, durvalumab, KY-1003, KD-033, MSB-0010718C, TSR-042, ALN-PDL, STI-A1014 and BMS-936559;

(34) PD-1 inhibitors selected from the group consisting of nivolumab, pembrolizumab, pidilizumab, BGB-108 and mDX-400;

(35) BTK inhibitors selected from the group consisting of ACP-196, dasatinib, ibrutinib, PRN-1008, SNS-062, ONO-4059, BGB-3111, MSC-2364447, X-022, spebrutinib, TP-4207, HM-71224, KBP-7536 and AC-0025;

(36) Other drugs for treating HBV selected from the group consisting of gentiopicrin (gentiopicroside), nitazoxanide, birinapant, NOV-205 (Molixan; BAM-205), Oligotide, Mivotilate, Feron, levamisole, Ka Shu Ning, Alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbama, IBPB-006IA, Hepuyinfen, DasKloster 0014-01, Jiangantai (Ganxikang), picroside, GA5 NM-HBV, DasKloster-0039, hepulantai, IMB-2613, TCM-800B, reduced glutathione and ZH-2N;

and

(37) The compounds disclosed in US20100143301 (Gilead Sciences), US20110098248 (Gilead Sciences), US20090047249 (Gilead Sciences), U.S. Pat. No. 8,722,054 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma). US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (VentirxPharma), US20140275167 (Novira therapeutics), US20130251673 (Novira therapeutics), U.S. Pat. No. 8,513,184 (Gilead Sciences), US20140030221 (Gilead Sciences), US20130344030 (Gilead Sciences), US20130344029 (Gilead Sciences), US20140343032 (Roche), WO2014037480 (Roche), US20130267517 (Roche), WO2014131847 (Janssen), WO2014033176 (Janssen), WO2014033170 (Janssen), WO2014033167 (Janssen), US20140330015 (Ono pharmaceutical), US20130079327 (Ono pharmaceutical), and US20130217880 (Ono pharmaceutical).

In certain embodiments, a compound disclosed herein, or a pharmaceutically acceptable salt thereof, is combined with one, two, three, four or more additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In a specific embodiment, a compound disclosed herein, or a pharmaceutically acceptable salt thereof, is combined with an HBV DNA polymerase inhibitor. In another specific embodiment, a compound disclosed herein, or a pharmaceutically acceptable salt thereof, is combined with an HBV DNA polymerase inhibitor and at least one additional therapeutic agent selected from the group consisting of: immunomodulators, toll-like receptor modulators (modulators of tlr1, tlr2, tlr3, tlr4, tlr5, tlr6, tlr7, tlr8, tlr9, tlr10, tlr11, tlr12 and tlr13), interferon alpha receptor ligands, hyaluronidase inhibitors, recombinant IL-7, HBsAg inhibitors, compounds targeting HbcAg, cyclophilin inhibitors, HBV therapeutic vaccines, HBV prophylactic vaccines HBV viral entry inhibitors, NTCP inhibitors, antisense oligonucleotide targeting viral mRNA, short interfering RNAs (siRNA), miRNA gene therapy agents, endonuclease modulators, inhibitors of ribonucleotide reductase, Hepatitis B virus E antigen inhibitors, recombinant scavenger receptor A (SRA) proteins, src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambda, recombinant thymosin alpha-1, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), and Hepatitis B virus replication inhibitors.

In another specific embodiment, a compound disclosed herein, or a pharmaceutically acceptable salt thereof, is combined with an HBV DNA polymerase inhibitor and at least a second additional therapeutic agent selected from the group consisting of: immunomodulators, toll-like receptor modulators (modulators of tlr1, tlr2, tlr3, tlr4, tlr5, tlr6, tlr7, tlr8, tlr9, tlr10, tlr11, tlr12 and tlr13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®), Fab derivatives), cyclophilin inhibitors, stimulators of retinoic acid-inducible gene 1, PD-1 inhibitors, PD-L1 inhibitors, Arginase-1 inhibitors, PI3K inhibitors and stimulators of NOD2.

In another specific embodiment, a compound disclosed herein, or a pharmaceutically acceptable salt thereof, is combined with an HBV DNA polymerase inhibitor and at least a second additional therapeutic agent selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In another specific embodiment, a compound disclosed herein, or a pharmaceutically acceptable salt thereof, is combined with an HBV DNA polymerase inhibitor, one or two additional therapeutic agents selected from the group consisting of: immunomodulators, toll-like receptor modulators (modulators of tlr1, tlr2, tlr3, tlr4, tlr5, tlr6, tlr7, tlr8, tlr9, tlr10, tlr11, tlr12 and tlr13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), cyclophilin inhibitors, stimulators of retinoic acid-inducible gene 1, PD-1 inhibitors, PD-L1 inhibitors, Arginase-1 inhibitors, PI3K inhibitors and stimulators of NOD2, and one or two additional therapeutic agents selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In a particular embodiment, a compound disclosed herein, or a pharmaceutically acceptable salt thereof, is combined with one, two, three, four or more additional therapeutic agents selected from adefovir (Hepsera®), tenofovir disoproxil fumarate+emtricitabine (Truvada®), tenofovir disoproxil fumarate (Viread®), entecavir (Baraclude®), lamivudine (Epivir-HBV®), tenofovir alafenamide, tenofovir, tenofovir disoproxil, tenofovir alafenamide fumarate, tenofovir alafenamide hemifumarate, telbivudine (Tyzeka®), Clevudine, emtricitabine (Emtriva®), peginterferon alfa-2b (PEG-Intron®), Multiferon®, interferon alpha 1b (Hapgen®), interferon alpha-2b (Intron A®), pegylated interferon alpha-2a (Pegasys®), interferon alfa-n1 (Humoferon®), ribavirin, interferon beta-1a (Avonex®), Bioferon, Ingaron, Inmutag (IFN), Algeron, Roferon-A, Oligotide, Zutectra, Shaferon, interferon alfa-2b (AXXO), Alfaferone, interferon alfa-2b (BioGeneric Pharma), Feron, interferon-alpha 2 (CJ), BEVAC, Laferonum, VIPEG, BLAUFERON-B, BLAUFERON-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products). Anterferon, Shanferon, MOR-22, interleukin-2 (IL-2, Immunex), recombinant human interleukin-2 (Shenzhen Neptunus), Layfferon, Ka Shu Ning, Shang Sheng Lei Tai, INTEFEN, SINOGEN, Fukangtai, Alloferon and celmoleukin;

In a particular embodiment, a compound disclosed herein, or a pharmaceutically acceptable salt thereof, is combined with entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®).

In a particular embodiment, a compound disclosed herein, or a pharmaceutically acceptable salt thereof, is combined with entecavir (Baraclude®), adefovir (Hepsera®), tenofovir disoproxil fumarate (Viread®), tenofovir alafenamide hemifumarate, telbivudine (Tyzeka®) or lamivudine (Epivir-HBV®).

In a particular embodiment, a compound disclosed herein, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least a second additional therapeutic agent selected from the group consisting of immunomodulators, toll-like receptor modulators (modulators of tlr1, tlr2, tlr3, tlr4, tlr5, tlr6, tlr7, tlr8, tlr9, tlr10, tlr11, tlr12 and tlr13), interferon alpha receptor ligands, hyaluronidase inhibitors, recombinant IL-7, HBsAg inhibitors, compounds targeting HbcAg, cyclophilin inhibitors, HBV Therapeutic vaccines, HBV prophylactic vaccines, HBV viral entry inhibitors, NTCP inhibitors, antisense oligonucleotide targeting viral mRNA, short interfering RNAs (siRNA), miRNA gene therapy agents, endonuclease modulators, inhibitors of ribonucleotide reductase, Hepatitis B virus E antigen inhibitors, recombinant scavenger receptor A (SRA) proteins, src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, recombinant thymosin alpha-1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors. PD-1 inhibitors, PD-L inhibitors, PEG-Interferon Lambd, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), a and Hepatitis B virus replication inhibitors.

In a particular embodiment, a compound disclosed herein, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least a second additional therapeutic agent selected from the group consisting of peginterferon alfa-2b (PEG-Intron®), Multiferon®, interferon alpha 1b (Hapgen®), interferon alpha-2b (Intron A®), pegylated interferon alpha-2a (Pegasvs®), interferon alfa-n1 (Humoferon®), ribavirin, interferon beta-1a (Avonex®), Bioferon, Ingaron, Inmutag (Inferon), Algeron, Roferon-A, Oligotide, Zutectra, Shaferon, interferon alfa-2b (AXXO), Alfaferone, interferon alfa-2b (BioGeneric Pharma), Feron, interferon-alpha 2 (CJ), BEVAC, Laferonum, VIPEG, BLAUFERON-B, BLAUFERON-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, MOR-22, interleukin-2 (IL-2, Immunex), recombinant human interleukin-2 (Shenzhen Neptunus). Layfferon, Ka Shu Ning, Shang Sheng Lei Tai, INTEFEN, SINOGEN, Fukangtai, Alloferon and celmoleukin;

In a particular embodiment, a compound disclosed herein, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least a second additional therapeutic agent selected from the group consisting of immunomodulators, toll-like receptor modulators (modulators of tlr1, tlr2, tlr3, tlr4, tlr5, tlr6, tlr7, tlr8, tlr9, tlr10, tlr11, tlr12 and tlr13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), cyclophilin inhibitors, stimulators of retinoic acid-inducible gene 1, Arginase-1 inhibitors, PI3K inhibitors, PD-1 inhibitors. PD-L1 inhibitors and stimulators of NOD2.

In a particular embodiment, a compound disclosed herein, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least a second additional therapeutic agent selected from the group consisting of HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In a particular embodiment, a compound disclosed herein, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®), one or two additional therapeutic agents selected from the group consisting of: immunomodulators, toll-like receptor modulators (modulators of tlr1, tlr2, tlr3, tlr4, tlr5, tlr6, tlr7, tlr8, tlr9, tlr10, tlr11, tlr12 and tlr13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), cyclophilin inhibitors, stimulators of retinoic acid-inducible gene 1, PD-1 inhibitors, PD-L1 inhibitors, Arginase-1 inhibitors, PI3K inhibitors and stimulators of NOD2, and one or two additional therapeutic agents selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In some embodiments, a compound disclosed herein, or a pharmaceutically acceptable salt thereof, is combined with tenofovir alafenamide. In some embodiments the tenofovir alafenamide may be tenofovir alafenamide monofumarate or tenofovir alafenamide hemifumarate. Typically, the tenofovir alafenamide is tenofovir alafenamide hemifumarate. In some embodiments the compound disclosed herein, or a pharmaceutically acceptable salt thereof and tenofovir alafenamide are administered to a subject separately. In other embodiments, the compound disclosed herein, or a pharmaceutically acceptable salt thereof and tenofovir alafenamide are administered to a subject in combination.

In certain embodiments, a compound disclosed herein, or a pharmaceutically acceptable salt thereof, is combined with 5-30 mg tenofovir alafenamide fumarate, tenofovir alafenamide hemifumarate, or tenofovir alafenamide. In certain embodiments, a compound disclosed herein, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In certain embodiments, a compound disclosed herein, or a pharmaceutically acceptable salt thereof, is combined with 10 mg tenofovir alafenamide fumarate, tenofovir alafenamide hemifumarate, or tenofovir alafenamide. In certain embodiments, a compound disclosed herein, or a pharmaceutically acceptable salt thereof, is combined with 25 mg tenofovir alafenamide fumarate, tenofovir alafenamide hemifumarate, or tenofovir alafenamide. A compound as disclosed herein (e.g., a compound of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2}) may be combined with the agents provided herein in any dosage amount of the compound (e.g., from 50 mg to 500 mg of compound) the same as if each combination of dosages were specifically and individually listed.

In certain embodiments, a compound disclosed herein, or a pharmaceutically acceptable salt thereof, is combined with 5-30 mg tenofovir alafenamide fumarate, tenofovir alafenamide hemifumarate, or tenofovir alafenamide. In certain embodiments, a compound disclosed herein, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In certain embodiments, a compound disclosed herein, or a pharmaceutically acceptable salt thereof, is combined with 10 mg tenofovir alafenamide fumarate, tenofovir alafenamide hemifumarate, or tenofovir alafenamide. In certain embodiments, a compound disclosed herein, or a pharmaceutically acceptable salt thereof, is combined with 25 mg tenofovir alafenamide fumarate, tenofovir alafenamide hemifumarate, or tenofovir alafenamide. A compound as disclosed herein (e.g., a compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, II^{b6}, I^{c} and I^{c2}) may be combined with the agents provided herein in any dosage amount of the compound (e.g., from 50 mg to 500 mg of compound) the same as if each combination of dosages were specifically and individually listed.

In some embodiments, a compound disclosed herein, or a pharmaceutically acceptable salt thereof, is combined with tenofovir disoproxil. In some embodiments the tenofovir disoproxil may be tenofovir disoproxil fumarate, tenofovir disoproxil phosphate or tenofovir disoproxil succinate. Typically, the tenofovir disoproxil is tenofovir disoproxil fumarate. In some embodiments the compound disclosed herein, or a pharmaceutically acceptable salt thereof and tenofovir disoproxil are administered separately. In other embodiments, the compound disclosed herein, or a pharmaceutically acceptable salt thereof and tenofovir disoproxil are administered in combination.

In certain embodiments, a compound disclosed herein, or a pharmaceutically acceptable salt thereof, is combined with 100-400 mg tenofovir disoproxil fumarate, tenofovir disoproxil hemifumarate, or tenofovir disoproxil. In certain embodiments, a compound disclosed herein, or a pharmaceutically acceptable salt thereof, is combined with 100-150; 100-200. 100-250, 100-300; 100-350; 150-200; 150-250; 150-300; 150-350; 150-400; 200-250; 200-300; 200-350; 200-400; 250-350; 250-400; 350-400 or 300-400 mg tenofovir disoproxil fumarate, tenofovir disoproxil hemifumarate, or tenofovir disoproxil. In certain embodiments, a compound disclosed herein, or a pharmaceutically acceptable salt thereof, is combined with 300 mg tenofovir disoproxil fumarate, tenofovir disoproxil hemifumarate, or tenofovir disoproxil. In certain embodiments, a compound disclosed herein, or a pharmaceutically acceptable salt thereof, is combined with 250 mg tenofovir disoproxil fumarate, tenofovir disoproxil hemifumarate, or tenofovir disoproxil. In certain embodiments, a compound disclosed herein, or a pharmaceutically acceptable salt thereof, is combined with 150 mg tenofovir disoproxil fumarate, tenofovir disoproxil hemifumarate, or tenofovir disoproxil. A compound as disclosed herein (e.g., a compound of Formulae I^{a}, I^{a2}, I^{b}, I^{b2}, II^{b2}, I^{b3}, I^{b3a}, I^{b3b}, II^{b3}, I^{b4}, I^{b5}, I^{b6}, II^{b6}, I^{c} and I^{c2}) may be combined with the agents provided herein in any dosage amount of the compound (e.g., from 50 mg to 500 mg of compound) the same as if each combination of dosages were specifically and individually listed.

In certain embodiments, a compound disclosed herein, or a pharmaceutically acceptable salt thereof, is combined with 100-400 mg tenofovir disoproxil fumarate, tenofovir disoproxil hemifumarate, or tenofovir disoproxil. In certain embodiments, a compound disclosed herein, or a pharmaceutically acceptable salt thereof, is combined with 100-150, 100-200, 100-250; 100-300; 100-350; 150-200; 150-250; 150-300; 150-350; 150-400; 200-250; 200-300, 200-350; 200-400; 250-350; 250-400; 350-400 or 300-400 mg tenofovir disoproxil fumarate, tenofovir disoproxil hemifumarate, or tenofovir disoproxil. In certain embodiments, a compound disclosed herein, or a pharmaceutically acceptable salt thereof, is combined with 300 mg tenofovir disoproxil fumarate, tenofovir disoproxil hemifumarate, or tenofovir disoproxil. In certain embodiments, a compound disclosed herein, or a pharmaceutically acceptable salt thereof, is combined with 250 mg tenofovir disoproxil fumarate, tenofovir disoproxil hemifumarate, or tenofovir disoproxil. In certain embodiments, a compound disclosed herein, or a pharmaceutically acceptable salt thereof, is combined with 150 mg tenofovir disoproxil fumarate, tenofovir disoproxil hemifumarate, or tenofovir disoproxil. A compound as disclosed herein (e.g., a compound of Formulae I^{a}, I^{a1}, I^{a2}, I^{b}, I^{b2}, II^{b2}, I^{b3}, I^{b3a}, I^{b3b}, II^{b3}, I^{b4}, I^{b5}, II^{b5}, I^{b6}, I^{c} and I^{c2}) may be combined with the agents provided herein in any dosage amount of the compound (e.g., from 50 mg to 500 mg of compound) the same as if each combination of dosages were specifically and individually listed.

In some embodiments, a compound disclosed herein, or a pharmaceutically acceptable salt thereof, is combined with a TLR8 inhibitor. In some embodiments the compound disclosed herein, or a pharmaceutically acceptable salt thereof and the TLR8 inhibitor are administered separately. In other embodiments, the compound disclosed herein, or a pharmaceutically acceptable salt thereof and TLR8 inhibitor are administered in combination.

In certain embodiments, when a compound disclosed herein is combined with one or more additional therapeutic agents as described above, the components of the composition are administered as a simultaneous or sequential regimen. When administered sequentially, the combination may be administered in two or more administrations.

In certain embodiments, a compound disclosed herein is combined with one or more additional therapeutic agents in a unitary dosage form for simultaneous administration to a patient, for example as a solid dosage form for oral administration.

In certain embodiments, a compound disclosed herein is administered with one or more additional therapeutic agents. Co-administration of a compound disclosed herein with one or more additional therapeutic agents generally refers to simultaneous or sequential administration of a compound disclosed herein and one or more additional therapeutic agents, such that therapeutically effective amounts of the compound disclosed herein and one or more additional therapeutic agents are both present in the body of the patient.

Co-administration includes administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound disclosed herein is administered first, followed within seconds or minutes by administration of a unit dose of one or more additional therapeutic agents. Alternatively, in other embodiments, a unit dose of one or more additional therapeutic agents is administered first, followed by administration of a unit dose of a compound disclosed herein within seconds or minutes. In some embodiments, a unit dose of a compound disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound disclosed herein.

Thus, the invention provides a product comprising one or more KDM5 inhibitors as disclosed herein and one or more additional therapeutic agents as a combined preparation for simultaneous, separate or sequential use in treating HBV.

The present invention is also directed to one or more KDM5 inhibitors as disclosed herein for use in methods of treating HBV according to any of the methods disclosed herein. In some embodiments, the invention is directed to one or more KDM5 inhibitors as disclosed herein for use in a method of treating HBV wherein said method further comprises administering one or more additional therapeutic agents as defined herein to the subject in need of treatment.

In further embodiments, the invention provides one or more KDM5 inhibitors as disclosed herein in combination with one or more additional therapeutic agents as defined herein for use in a method of treating HBV. Also provided is one or more additional therapeutic agents as defined herein for use in a method of treating HBV, the method further comprising administering one or more KDM5 inhibitors to the subject in need of treatment.

### EXAMPLES

### Example 1. Synthesis of the Compounds

Compounds were made according to the sources identified in Table 1.

TABLE 1
Representative Compounds

| Example | Structure | Name/Source |
|---|---|---|
| 1 | 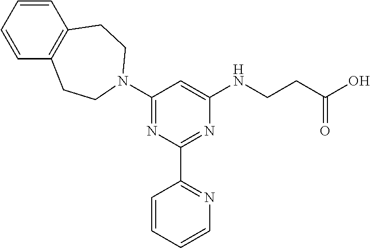 [SMILES: O=C(O)CCNc1cc(N2CCc3ccccc3CC2)nc(-c2ccccn2)n1] | GSK-J1/Tocris Biosciences, Cat. No 4593 |
| 2 | 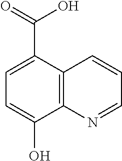 [SMILES: O=C(O)c1ccc(O)c2ncccc12] | KDM4/6 inhibitor 8- HQ-5-COOH/Tocris Biosciences, Cat. No. 4464 |
| 3 | 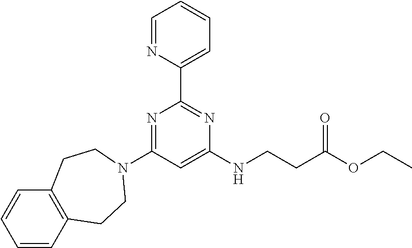 [SMILES: CCOC(=O)CCNc1cc(N2CCc3ccccc3CC2)nc(-c2ccccn2)n1] | GSK-J4 KDM5B inhibitor/Tocris Biosciences, Cat. No. 4594 |
| 4 | 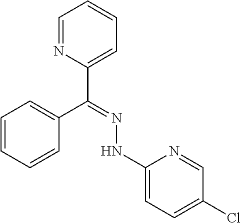 [SMILES: Clc1ccc(N/N=C(\c2ccccc2)c2ccccn2)nc1] | JIB-04, pan KDM inhibitor, (NSC693627);/Tocris Biosciences, Cat. No. 4972 |
| 5 | 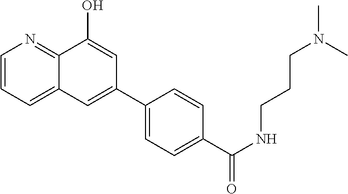 [SMILES: CN(C)CCCNC(=O)c1ccc(-c2cc(O)c3ncccc3c2)cc1] | ML-324, KDM4/Axon MedChem, Cat. No. 2081 |
| 6 | 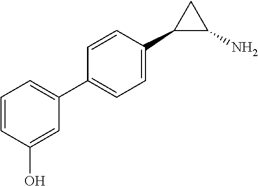 [SMILES: N[C@H]1C[C@@H]1c1ccc(-c2cccc(O)c2)cc1] | OG-L002 KDM1A/Axon MedChem, Cat. No. 2077 |
| 7 | 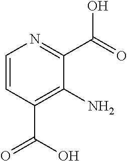 [SMILES: Nc1c(C(=O)O)ccnc1C(=O)O] | 2-amino-2,4-PDCA, pan KDM/Anichem, Cat. No. NC5561 |
| 8 | 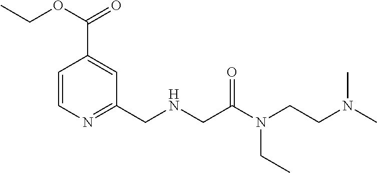 [SMILES: CCOC(=O)c1ccnc(CNCC(=O)N(CC)CCN(C)C)c1] | KDM5 Epitherapeutics WO 2014053491/WO 2014053491 example 65 |
| 9 | 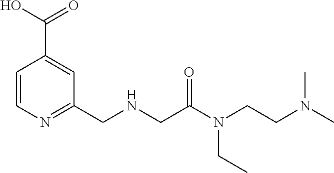 [SMILES: CCN(CCN(C)C)C(=O)CNCc1cc(C(=O)O)ccn1] | KDM Epitherapeutics WO 2014053491/WO 2014053491 example 49 |
| 10 | 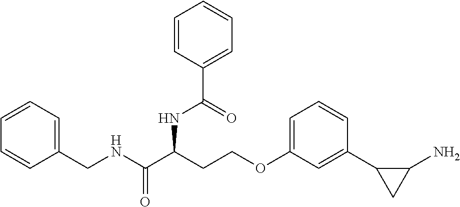 [SMILES: NC1CC1c1cccc(OCC[C@H](NC(=O)c2ccccc2)C(=O)NCc2ccccc2)c1] | NCL-1 KDM1A/TCI America, Cat No. A2411 |
| 11 | 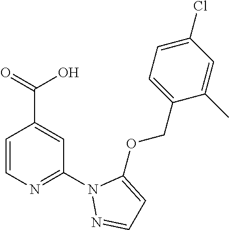 [SMILES: Cc1cc(Cl)ccc1COc1ccnn1-c1cc(C(=O)O)ccn1] | Quanticel, KDM5/WO 2014089364 example 89 |
| 12 | 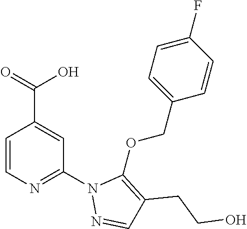 [SMILES: O=C(O)c1ccnc(-n2ncc(CCO)c2OCc2ccc(F)cc2)c1] | Quanticel, KDM5/WO 2014089364 example 109 |
| 13 | 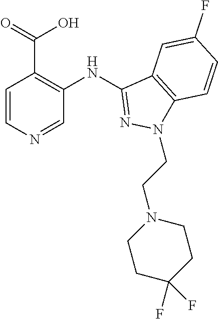 [SMILES: O=C(O)c1ccncc1Nc1nn(CCN2CCC(F)(F)CC2)c2ccc(F)cc12] | Quanticel, KDM5/WO 2014100463 example 73 |
| 14 | 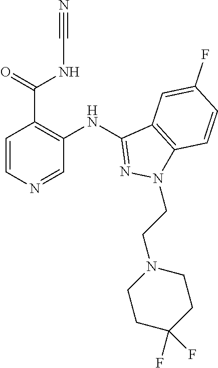 [SMILES: N#CNC(=O)c1ccncc1Nc1nn(CCN2CCC(F)(F)CC2)c2ccc(F)cc12] | Quanticel, KDM5/WO 2014100463 example 74 |
| 15 | 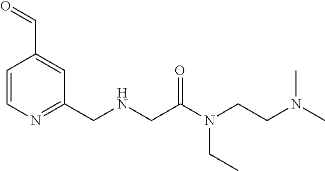 [SMILES: CCN(CCN(C)C)C(=O)CNCc1cc(C=O)ccn1] | Epitherapeutics KDM5/WO 2014131777 example 101 |
| 16 | 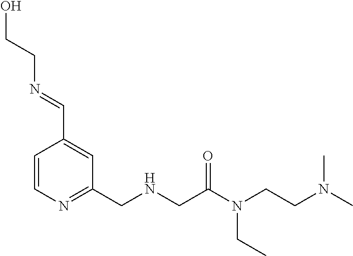 [SMILES: CCN(CCN(C)C)C(=O)CNCc1cc(/C=N/CCO)ccn1] | Epitherapeutics KDM5/WO 2014131777 example 66 |
| 18 | 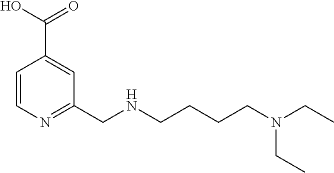 [SMILES: CCN(CC)CCCCNCc1cc(C(=O)O)ccn1] | Epitherapeutics KDM5/WO 2014053491 example 48 |
| 21 | 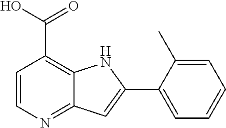 [SMILES: Cc1ccccc1-c1cc2nccc(C(=O)O)c2[nH]1] | Quanticel, KDM5/WO2014164708 (10/09) example 3 |
| 22 | 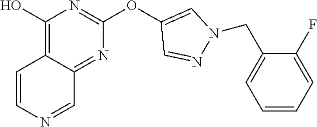 [SMILES: Oc1nc(Oc2cnn(Cc3ccccc3F)c2)nc2cnccc12] | Quanticel, KDM5/WO2014151106 (09/25) example 87 |
| 23 | 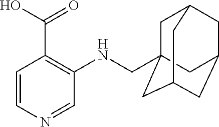 [SMILES: O=C(O)c1ccncc1NCC12CC3CC(CC(C3)C1)C2] | Quanticel, KDM5/WO2014100818 (06/26) example 42 |
| 24 | 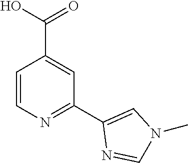 | Quanticel, KDM5/WO2014151945 (9/25) example 1 |
| 25 | 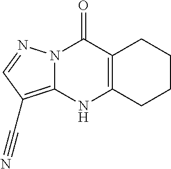 [SMILES: N#Cc1cnn2c(=O)c3c([nH]c12)CCCC3] | Constellation, KDM5/US 20140275092 I-4 |
| 26 | 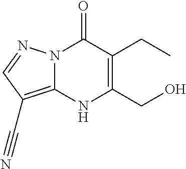 [SMILES: CCc1c(CO)[nH]c2c(C#N)cnn2c1=O] | Constellation, KDM5/US 20140275092 I-21 |
| 27 | 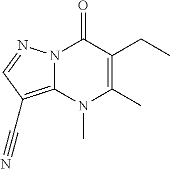 [SMILES: CCc1c(C)n(C)c2c(C#N)cnn2c1=O] | Constellation, KDM5/US 20140275092 I-30 |
| 28 | 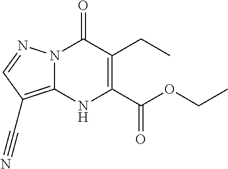 [SMILES: CCOC(=O)c1[nH]c2c(C#N)cnn2c(=O)c1CC] | Constellation, KDM5/US 20140275092 I-25 |
| 29 | 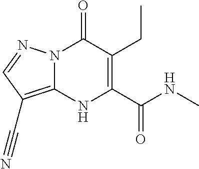 [SMILES: CCc1c(C(=O)NC)[nH]c2c(C#N)cnn2c1=O] | Constellation. KDM5/US 20140275092 I-49 |
| 30 | 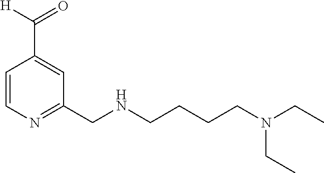 [SMILES: CCN(CC)CCCCNCc1cc(C=O)ccn1] | Epitherapeutics, KDM5/WO 2014131777 example 107 |
| 31 | 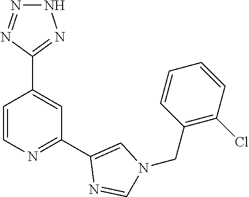 [SMILES: Clc1ccccc1Cn1cnc(-c2cc(-c3nn[nH]n3)ccn2)c1] | Quanticel, KDM5/WO2014151945 (9/25) example 129 |
| 32 | 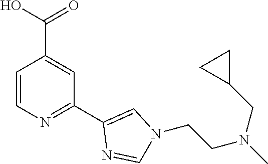 [SMILES: CN(CCn1cnc(-c2cc(C(=O)O)ccn2)c1)CC1CC1] | Quanticel, KDM5/WO2014151945 (9/25) example 59 |
| 33 | 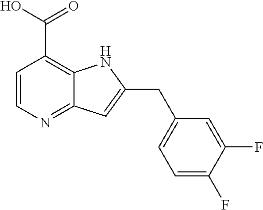 [SMILES: O=C(O)c1ccnc2cc(Cc3ccc(F)c(F)c3)[nH]c12] | Quanticel, KDM5/WO2014164708 (10/09) example 126 |
| 34 | 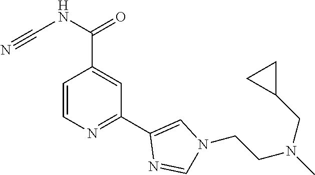 [SMILES: CN(CCn1cnc(-c2cc(C(=O)NC#N)ccn2)c1)CC1CC1] | Quanticel, KDM5/WO2014151945 (9/25) example 64 |
| 35 | 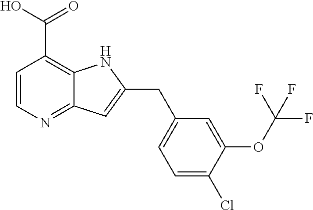 [SMILES: O=C(O)c1ccnc2cc(Cc3ccc(Cl)c(OC(F)(F)F)c3)[nH]c12] | Quanticel, KDM5/WO2014164708 (10/09) example 142 |
| 36 | 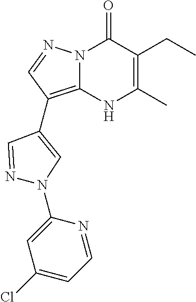 [SMILES: CCc1c(C)[nH]c2c(-c3cnn(-c4cc(Cl)ccn4)c3)cnn2c1=O] | Constellation, WO2015035062 example 13 |

### Example 2. Biochemical KDM Inhibition Assays

Representative compounds were characterized for their inhibition of KDM5 using biotinylated histone substrates. Inhibition was measured in vitro (CEREP Poitier, Le Bois l'Evêque, France) following experimental conditions described in Table 2. Briefly, the test compound, reference compound or water (control) was mixed with about 2-20 ng of recombinant, Human enzymes expressed in Sf9 cells in a buffer containing 45 mM Hepes/Tris (pH 7), 5 μM FAS, 100 μM ascorbic acid, 10 μM 2-oxoglutarate, 0.01% Tween 20 and 0.01% BSA. Thereafter, the reaction was initiated by adding the biotin-labeled substrate, and the mixture was incubated for 10-30 min at room temperature. For basal control measurements, the enzyme was omitted from the reaction mixture. Following incubation the reaction was stopped by adding 1 mM EDTA. After 5 min, the anti-methyl histone antibody labeled with europium chelate and the Ulight streptavidine (Perkin Elmer Waltham, Massachusetts) were added. After 60 min more, the fluorescence transfer was measured at λₑₓ=320 nm and λₑₘ=620 and λₑₘ=665 nm using a microplate reader (Envision, Perkin Elmer) (LANCE). The enzyme activity was determined by dividing the signal measured at λₑₘ=665 nm by that measured at 620 nm (ratio).

TABLE 2
Reagents and Conditions for biochemical KDM inhibition assays

| Enzyme | Substrate (conc) | Incubation | Measured Product | Reference |
|---|---|---|---|---|
| KDM5C | Biotin-H3K4me3 (15 nM) | 10 min RT | Biotin- H3K4me2 | 1 |
| KDM5D | Biotin-H3K4me3 (100 nM) | 10 min RT | Biotin-H3K4me2 | 1 |
| KDM5A | biotin-H3K4me3 (100 nM) | 10 min RT | Biotin-H3K4me2 | 1 |
| KDM5B | Biotin-H3K4me3 (60 nM) | 30 min RT | Biotin-H3K4me2 | 2 |
| KDM6A | Biotin-H3K4me1 (150 nM) | 30 min RT | Biotin-H3K4 | 3 |
| KDM2A | Biotin-H3K27Me3 (50 nM) | 10 min RT | Biotin-H3K27Me2 | 4 |
| KDM2B | biotin-H3K36me2 (50 nM) | 10 min RT | Biotin-H3K36me1 | 5 |
| KDM3A | biotin-H3K36me2 (24 nM) | 10 min RT | Biotin-H3K36me1 | 3 |
| KDM4A | biotin-H3K9Mel (25 nM) | 10 min RT | Biotin-H3K9 | 6 |
| KDM4C | biotin-H3K9Me3 (100 nM) | 10 min RT | Biotin-H3K9Me2 | 7 |
| KDM4E | biotin-H3K9me3 (150 nM) | 15 min RT | Biotin-H3K9me2 | 8 |
| KDM6B | biotin-H3K9Me3 (300 nM) | 10 min RT | Biotin-H3K9Me2 | 9 |
| KDM5C | biotin H3K27Me3 (200 nM) | 10 min RT | Biotin H3K27Me2 | 10 |

7. King O. N. F. et al. (2010), PLoS ONE, 5: 1-12; 6. Heightman T. D. (2011), Current Chemical Genomics, 5: 62-71; 8. Yu V. et al. (2011). J Biomol Screen, 17: 27-38; 9. Thalhammer A. et al. (2011), Org. Biomol Chem., 9: 127-135; 1. NOTTKE, A. et al. (2009), Development, 136: 879-889; 3. ROTILI, D. and MAI, A. (2011), Genes & Cancer. 2: 663-679, 5. CHOWDHURY, R. et al. (2011), Eur. Mol. Biol. Org., 12: 463-469; 4. Hong, S. et al. (2007), PNAS, 104: 18439-18444. 2. Kristensen. L. H. et al. (2012), FEBS Journal, 279: 1905-1914; 10. Xiang Y, Zhu Z, Han G, Lin H, Xu L, Chen C D. (2007), Cell Res. 17 (10):850-7.

Table 3 summarizes inhibitory potency of structurally diverse toward members of Jumonji family of histone demethylases.

TABLE 3
Biochemical IC₅₀s (μM) of selected KDM inhibitors for various KDMs

| | KDM | | | | | | | | | | | |
|---|---|---|---|---|---|---|---|---|---|---|---|---|
| Example | 5C | 5D | 5A | 5B | 6A | 2A | 2B | 3A | 4A | 4C | 4E | 6B |
| 7 | 0.54 | 0.7 | 1.2 | 0.51 | 0.93 | 0.36 | 0.16 | 1.2 | 0.49 | 0.11 | 0.064 | 7.8 |
| 9 | 0.026 | 0.066 | 0.024 | 0.014 | 1.3 | 0.65 | 1.6 | 1.1 | 0.35 | 0.057 | 0.035 | 4.1 |
| 11 | 0.27 | 0.098 | 0.028 | 0.036 | 0.074 | 0.45 | 0.66 | 7.5 | 1 | 0.11 | 0.11 | 0.28 |
| 12 | | 6.4 | 1.6 | 2.7 | | | | | | | 9.1 | |
| 13 | 0.086 | 0.065 | 0.021 | 0.021 | 0.14 | 0.51 | 2.4 | | 1.1 | 0.26 | 0.67 | 0.4 |
| 14 | 0.24 | 0.085 | 0.029 | 0.014 | | | | 3.9 | | 3.1 | 2.3 | |
| 18 | 0.021 | 0.033 | 0.011 | 0.0031 | 5.5 | 2 | 12 | 5.4 | 0.39 | 0.35 | 1.2 | 27 |

### Example 3. Western Blot Protocol and Detection of H3K4Me3 in PHHs

One million primary human hepatocytes (PHH) cells from three different donors were plated in 6 well collagen coated tissue culture plates in 2.5 ml Plating Media containing William's Medium E supplemented with 1% Penicillin/Streptomycin, 4 μg/mL human recombinant insulin, 2 mM glutamax, 15 mM Hepes, 1 μM dexamethasone and 5% fetal bovine serum (Life Technologies, Cat#A12176-01 Life Technologies, Chicago, Ill.) and incubated for 4-hours at 37° C. Following this incubation the media was changed to Maintenance Media (Cat#CM4000-A15564 Life Technologies. Chicago, Ill.) containing William's Medium E supplemented with 0.5% Penicillin/Streptomycin, 6.25 μg/mL human recombinant insulin, 6.25 μg/mL human transferrin, 6.25 ng/mL selenous acid, 1.25 mg/mL bovine serum albumin, 5.35 μg/mL linoleic acid, 2 mM glutamax, 15 mM Hepes, 0.1 μM dexamethasone, 2% fetal bovine serum, and 2% DMSO (Cat#D2650 Sigma, St. Louis, Mo.). The next day, cells were infected with approximately 500 genome equivalents of HBV clinical isolates 21P (GTA) or AD38 (GTD) per cell in Maintenance Media supplemented with 4% PEG 8000. Small molecule inhibitors targeting KDMs were serially diluted in Maintenance Media and added to cells at 3 days post infection (p.i.). Media with compounds was replenished every 2-3 days. Cells were harvested on day 14 p.i. by scraping the monolayer into ice-cold PBS supplemented with 5 mM Sodium Butyrate and concentrated by centrifugation at 1000×g for 5 minutes at 4° C. The cell pellets were washed twice by re-suspension in PBS and concentrated by centrifugation. The cells were lysed by suspension in Triton Extraction Buffer (TEB:PBS containing 0.5% Triton X 100 (v/v), 2 mM phenylmethylsulfonyl fluoride (PMSF), 0.02% (w/v) NaN₃) at a cell density of 10 cells per ml and incubated on ice for 10 minutes with gentle stirring. Following centrifugation at 500 g for 10 minutes at 4° C., the supernatants were removed and the pellets were washed in 5×10⁷ cells per ml TEB buffer and centrifuged as before. The pellets were re-suspended in 0.2N HCl at a cell density of 4×10⁷ cells per ml and the histones were acid extracted overnight at 4° C. Samples were centrifuged at 500 g for 10 minutes at 4° C., the supernatants were removed and protein content was determined using the Bradford assay. Histones were separated on a 4-20% gradient SDS gel (Mini protean TGX precast gels from BioRad), and blotted to a Hybond C-extra nitrocellulose membrane (Amersham Biosciences, RPN303E). H3K4me3 and total H3 were detected with a mixture of 10 antibodies (Cat#05-745R Millipore and Cat#14269S Cell Signaling) diluted 1:1000 in 5% skimmed milk powder in PBS containing 0.1% Tween. The western blot was washed 3 times in PBS containing 0.1% tween and incubated with 20 antibodies (Donkey anti-Mouse IRDye at 680LT Cat#926-68022 Licor Odyssey; Donkey anti-Rabbit IRDye at 800CW Cat#96-32213; Licor Odyssey) diluted 1:10000 in 5% skimmed milk powder in PBS containing 0.1% tween 20 for 1 h. Detection of infrared fluorescence was performed on Infrared Fluorescence Imaging System LI-COR. Concentration dependence of H3K4me3/H3 signal was used to calculate IC₅₀ value for induction of H3K4 trimethylation.

### Example 4. Effect of KDM5 Inhibitors on H3K4Me3 Methylation Mark

Primary Human Hepatocytes (PHH) from three different donors, treated with Examples 8, 9, and 15 showed an increase in the chromatin H3K4Me3 mark in a dose-dependent manner consistent with the ability of the parent compound Example 9 to inhibit the KDM5 subfamily of histone demethylases (Table 4). The IC₅₀ values for compound-dependent inhibition of H3K4me3 demethylation were similar between PHH donors and did not depend on the type of virus used for HBV infection.

TABLE 4
Effect of KDM inhibitors on the intracellular levels of H3K4me3
mark in various PHH donors infected with HBV AD38 or 21P

| | IC₅₀ (μM)^{a} | | | | | |
|---|---|---|---|---|---|---|
| | Virus AD38 (GTD) | | | Virus 21P (GTA) | | |
| | | PHH | | | PHH | |
| Example | PHH 4239 | 8130 | PHH 8181 | PHH 4239 | 8130 | PHH 8181 |
| 8 | 0.03 | 0.003 | 0.05 | 0.03 | 0.001 | 0.07 |
| 9 | 0.01 | 0.13 | 0.13 | 0.06 | 0.13 | 0.1 |
| 15 | 0.02 | 0.05 | 0.04 | nd | 0.05 | 0.03 |

Infected PHH were treated with increasing concentration of compounds for up to 14 days. The compounds were added on day 0 and replenished on days 3 and 6. Histones were extracted from cells and ratio of chromosomal H3K4Me3/H3K4 was determined by Western blot analysis using antibodies specific to H3K4Me3 and H3K4.

^{a}IC₅₀ indicates the concentration of the tested compound causing a 50% increase in the H3K4me3 mark

### Example 5. PHH Screening Protocol

HBV antiviral activity was assessed in primary human hepatocytes (PHH) in a 96-well format. PHH were (Life Technologies, Chicago, Ill.) plated on collagen coated tissue culture plates using Plating Media containing William's Medium E supplemented with 1% Penicillin/Streptomycin, 4 μg/mL human recombinant insulin, 2 mM glutamax, 15 mM Hepes, 1 μM dexamethasone and 5% fetal bovine serum (Life Technologies, Cat#A12176-01 Life Technologies, Chicago, Ill.). After a 4-hour incubation at 37° C., cells were switched to Maintenance Media (Cat#CM4000-A15564 Life Technologies, Chicago, Ill.) containing William's Medium E supplemented with 0.5% Penicillin/Streptomycin, 6.25 μg/mL human recombinant insulin, 6.25 μg/mL human transferrin, 6.25 ng/mL selenous acid, 1.25 mg/mL bovine serum albumin, 5.35 μg/mL linoleic acid, 2 mM glutamax, 15 mM Hepes, 0.1 μM dexamethasone, 2% fetal bovine serum, and 2% DMSO (Cat#D2650 Sigma. St. Louis, Mo.). On the next day, cells were infected with approximately 500 genome equivalent of selected HBV clinical isolates (21P (GTA), 32P (GTA), 91P (GTA), AD38 (GTD), 65P (GTD) or 30P (GTE); ProteoGenex, Culver City, Calif.) per cell in Maintenance Media supplemented with 4% PEG 8000 (Cat#V3011 Promega, Madison, Wis.). After 24 hour incubation cells were washed three times with William's Medium E and fed with fresh Maintenance Media. Small molecule inhibitors targeting KDMs were serially diluted in Maintenance Media and added to cells at 3 days post infection (p.i.). Media with compounds was replenished every 2-3 days. Media collected on various days was used for determination of HBsAg and HBeAg levels by MSD ELISA, and HBV RNA by qPCR All data were converted into percentages of the untreated control and non-linear regression was performed to calculate EC₅₀ or CC₅₀ values.

### Example 6. Cell Viability Assay

Alamar Blue cell viability reagent (Cat#DAL1100 Life Technologies, Chicago, Ill.) was diluted 1 to 10 in Maintenance Media and added to the cells. Cells were incubated for 4 h at 37° C. and the fluorescence signal, which is proportional to the number of live cells, was read using a fluorimeter with excitation/emission spectra set at 560/590 nm, respectively. Data were converted into percentages of the untreated control and non-linear regression was performed to calculate CC₅₀ values.

### Example 7. Determination of HBV Viral RNA

Following the Alamar Blue measurement, media was removed and total RNA from the cells was isolated using the RNeasy 96 Kit (Cat#74182 Qiagen, Venlo, Netherlands). HBV mRNA levels from Total RNA isolations were measured by RT-qPCR using the TaqMan Fast Virus 1-Step Master Mix (Cat#4444436 Life Technologies, Chicago Ill.) with primers specific to the HBx region (forward: 5′-CCG TCT GTG CCT TCT CAT CTG-3′ (SEQ ID NO: 9), reverse: 5′-AGT CCA AGA GTY CTC TTA TGY AAG ACC TT-3′ (SEQ ID NO: 10), probe: 5′-FAM-CC GTG TGC ACT TCG CTT CAC CTC TGC-BHQ1-3′ (SEQ ID NO: 11)) that should amplify all four HBV mRNA transcripts. GAPDH mRNA levels were also measured by RT-qPCR to control for differences in cell number, toxicity, and RNA purification efficiency (Cat#4390849 Life Technologies, Chicago Ill.). HBV mRNA Ct values were normalized using their cognate GAPDH mRNA Ct values by the delta-delta-Ct calculation and then expressed as a percentage of the non-targeting scrambled control. To validate siRNA knockdown of target transcripts, KDM5 mRNA levels were measured by RT-qPCR with the following primers: KDM5A Hs00231908_m1, KDM5B Hs00981910_m1, KDM5C Hs01011846_m1, KDM5D Hs00190491_m1 (Life Technologies, Chicago Ill.).

TABLE 5
Summary of HBV Antiviral Activity (nM)

| Example | HBsAg EC₅₀ | HBeAg EC₅₀ | HBV RNA EC₅₀ | PHH CC₅₀ |
|---|---|---|---|---|
| 1 | 22381 | 8044 | | 100000 |
| 2 | 45831 | 31957 | | 100000 |
| 3 | 28673 | 3432 | | 100000 |
| 4 | 900 | 900 | 4600 | 100000 |
| 5 | 38425 | 34506 | | 69428 |
| 6 | 38300 | 46700 | 31558 | 100000 |
| 7 | 481 | 642 | 224 | 100000 |
| 8 | 67 | 188 | 100 | 28231 |
| 9 | 2024 | 1631 | | 50000 |
| 10 | 19360 | 18953 | | 8118 |
| 11 | 4049 | 5771 | | 50000 |
| 12 | 50000 | 50000 | | 50000 |
| 13 | 3984 | 5968 | | 50000 |
| 14 | 1298 | 1090 | | 50000 |
| 15 | 41 | 53 | | 50000 |
| 16 | 257 | 648 | | 50000 |
| 18 | 19502 | 22632 | | |

### Example 8. RNAi Protocol

PHH were plated in collagen coated tissue culture plates in Plating Media and after 4-hour incubation at 37° C., cells were switched to Maintenance Media. On the next day, the cells were infected with 500 genome equivalents per cell of Genotype-A clinical isolate 21P (ProteoGenex. Culver City, Calif.) in 100l Maintenance Media supplemented with 4% PEG 8000 (Cat#V3011 Promega. Madison, Wis.). After an overnight incubation the inoculum was removed and the cells were washed three times with William's Media E and maintained in Maintenance Media. At three days post-infection, cells were transfected with 10 nM or 20 nM siRNAs (Cat# s11836, s21145, s15748, s15775; Life Technologies, Chicago, Ill.) targeting individual KDM genes or a combination of KDM5 members (A, B, C, or D) using RNAiMax (Cat#13778075 Life Technologies, Chicago, Ill.) transfection reagent. A non-targeting scrambled siRNA control (Cat#4390843 Life Technologies, Chicago, Ill.) was transfected at 40 nM to control for transfection and non-specific siRNA-related effects on HBV replication. Following the transfection, the cells were incubated at 37° C. in a humidified incubator and media was changed every 3-4 days. The assay was terminated on day 14 post infection and cell viability was assessed by Alamar Blue. The collected medium was used for determination of HBsAg and HBeAg levels by MSD ELISA while cells were processed for determination of HBV RNA using qPCR.

TABLE 6
KDM5 targeting siRNA

| Target | siRNA | | |
|---|---|---|---|
| mRNA | ID# | Sense (5′-3′) | Antisense (5′-3′) |
| KDM5a | s11836 | GCGAGUUUGUUGUGACAUUTT | AAUGUCACAACAAACUCGCCA |
| | | (SEQ ID NO: 1) | (SEQ ID NO: 5) |
| KDM5b | s21145 | GGCAGUAAAGGAAAUCGAATT | UUCGAUUUCCUUUACUGCCGT |
| | | (SEQ ID NO: 2) | (SEQ ID NO: 6) |
| KDM5c | s15748 | CAGACGAGAGUGAAACUGATT | UCAGUUUACUCUCGUCUGGG |
| | | (SEQ ID NO: 3) | (SEQ ID NO: 7) |
| KDM5d | s15775 | CAACCAUGCAACUUCGAAATT | UUUCGAAGUUGCAUGGUUGTC |
| | | (SEQ ID NO: 4) | (SEQ ID NO: 8) |

### Example 9. Effect of KDM5 RNAi on HBV Replication

Simultaneous knock-down of all four members of KDM5 subfamily of histone demethylases in PHHs using siRNA resulted in profound suppression of vRNA, HBsAg and HBeAg in PHH infected with patient virus 21P (Table 7 and 8). Single knock-down of individual KDM5s had no effect on HBV replication. Altogether these data indicate that inhibition of KDM5 subfamily of histone demethylases results in inhibition of HBV replication.

TABLE 7
Effects of KDM5 siRNA treatment on HBV
mRNA, HBeAg, and HBsAg production

| | HBV | | | GAPDH | Alamar |
|---|---|---|---|---|---|
| | mRNA | HBeAg | HBsAg | mRNA | Blue |
| KDM5a-d siRNA | 14% | 7% | 7% | 111% | 91% |
| 40 nM | | | | | |
| KDM5a-d siRNA | 22% | 10% | 10% | 96% | 84% |
| 80 nM | | | | | |
| Scrambled Control | 110% | 100% | 100% | 102% | 100% |

% calculated relative to non-targeting scrambled control determined on day 17 post transfection. GAPDH and Alamar blue assay are used as a toxicity control

TABLE 8
KDM5 mRNA Levels post-siRNA knockdown

| | KDM5A | KDM5B | KDM5C | KDM5D |
|---|---|---|---|---|
| KDM5A-D | 58% | 42% | 12% | 34% |
| siRNA 40 nM | | | | |
| KDM5A-D | 51% | 48% | 20% | 40% |
| siRNA 80 nM | | | | |
| Scrambled Control | 102% | 100% | 95% | 101% |

% calculated relative to non-targeting scrambled control determined on day 17 post transfection

### Activity of KDMi Using Various HBV Genotypes and PHH Donors

Activity of Example 8 was dependent on the PHH donor with donor 8181 being the most susceptible to KDM-dependent inhibition of HBV replication (Table 9). In this donor, Example 8 inhibited HBV HBsAg and HBeAg secretion by more than 10 fold compared to untreated cells with EC₅₀ values ranging from 0.02 to <0.002 μM. Donors 4239 and 8130 were less susceptible to Example 8 with EC₅₀ values ranging from 0.03 to 2.4 μM.

TABLE 9
Activity of Example 8 on HBsAg secretion
across Different Viruses and PHH Donors

| | HBsAg EC₅₀ (μM)^{a} | | |
|---|---|---|---|
| Patient HBV viruses | PHH 4239 | PHH 8130 | PHH 8181 |
| 21P (GTA) | 0.7 | 0.2 | 0.02 |
| 32P (GTA) | 0.03 | 0.08 | <0.002 |
| 91P (GTA) | 0.06 | 0.03 | <0.006 |
| AD38 (GTD) | 2 | 1.4 | <0.005 |
| 65P (GTD) | 0.3 | 2.4 | <0.002 |
| 30P (GTE) | 0.3 | 0.2 | <0.005 |

PHH donors 4239, 8130 and 8181 were infected with patient viruses for three days before serially diluted Example 8 was added to the cells. Activity of Example 8 was monitored on day 17 p.i. using HBsAg readout. The compounds and medium were replenished every 3-4 days in all experiments.

^{a}EC₅₀ indicates the concentration of Example 8 causing inhibition of HBsAg secretion into medium by HBV infected cells by 50%

### Example 10. Time Dependency of Anti HBV Activity of KDM Inhibitors

The data shown in Table 10 demonstrate that activity of Examples 7, 8, and 9 in PHH donor 8181 infected with patient virus 21p (GTA) was time dependent and the potency of the compound increased with incubation time. Similar observations was made for Example 8 in donors 4239 and 8130 infected with AD38 or 30P HBV viruses (Table 11); respectively.

TABLE 10
Time dependency of anti HBV activity of KDM inhibitors

| | | | | | | | | CC₅₀ |
|---|---|---|---|---|---|---|---|---|
| | | EC₅₀ (μM) | | | | | | (μM) |
| Example | | D8 | D13 | D17 | D22 | D27 | D31 | D28 |
| 7 | HBeAg | >50 | 4.03 | 1.10 | 0.09 | 0.06 | 0.06 | >50 |
| | HBsAg | >50 | 0.99 | 0.58 | 0.07 | 0.06 | 0.06 | >50 |
| 8 | HBeAg | >50 | 0.16 | <0.02 | <0.02 | <0.02 | <0.02 | 20 |
| | HBsAg | >50 | 0.15 | <0.02 | <0.02 | <0.02 | <0.02 | 20 |
| 9 | HBeAg | >100 | 0.849 | 0.267 | 0.153 | 0.3 | nd | >100 |
| | HBsAg | >100 | 2.334 | 0.369 | 0.2 | 0.4 | nd | >100 |

PHH donor 8181 was infected with patient virus 21p for three days before serially diluted Examples 7, 8, and 9 were added to the cells. Activity of compounds was monitored by measuring the effects of compound on HBsAg and HBeAg secretion. The compounds and medium were replenished every 3-4 days in all experiments.

TABLE 11
Time dependency of anti HBV activity of Example 8

| HMV | | | EC₅₀ (μM) | | | | |
|---|---|---|---|---|---|---|---|
| virus | PHH donor | | d14 | d17 | d20 | d25 | d31 |
| AD38 | 4239 | HBeAg | >15 | 1.66 | 0.67 | 0.11 | 0.20 |
| | | HBsAg | 6.303 | 0.842 | 0.300 | 0.372 | 0.152 |
| | 8130 | HBeAg | 2.42 | 0.84 | 0.19 | 0.03 | 0.01 |
| | | HBsAg | 2.006 | 0.920 | 0.081 | 0.080 | 0.012 |
| 30P | 4239 | HBeAg | 4.308 | 0.141 | 0.111 | 0.015 | 0.036 |
| | | HBsAg | >15 | 0.207 | 0.026 | 0.028 | 0.032 |
| | 8130 | HBeAg | 0.318 | 0.024 | 0.020 | 0.002 | 0.002 |
| | | HBsAg | 0.001 | 0.019 | 0.003 | 0.002 | 0.002 |

PHH donors 4239 and 8130 were infected with viruses AD38 or 30p for three days before serially diluted Example 8 was added to the cells. After 14 days of the treatment, the compound was removed and cells were followed for another 14 days. Activity of Example 8 was monitored by measuring the effects of compound on HBsAg and HBeAg secretion. The medium with/without compound was replenished every 3-4 days.

### Effect of the Withdrawal of KDM Inhibitors on HBV Rebound

PHH from donor 8181 infected with 21p virus was treated with serially diluted Examples 8 or 7 for 14 days. Afterwards the compound was removed and cell cultures were replenished regularly with fresh medium but without compound for another 14 days. The levels of HBsAg and HBeAg secretion were measured during the course of the experiment to monitor the effects of compound on virus replication. No rebound of HBsAg or HBeAg secretion into media was observed after the compound withdrawal. As shown in Tables 12 and 13, 0.08 μM of Example 8 and 2 μM Example 7 caused prolonged suppression of viral transcription after its withdrawal for up to another 14 days.

TABLE 12
Effect of removal of 0.08 μM of
Example 8 on HBsAg and HBeAg levels

| | |
|---|---|
| HBsAg (ng/mL) | HBeAg (ng/mL) |

| Day | 0 nM | 20 nM | 0 nM | 20 nM |
|---|---|---|---|---|
| 0 | 0 | 0 | 0 | 0 |
| 5 | 14 | 13.8 | 5.9 | 5.3 |
| 10 | 198 | 147 | 44 | 31 |
| 14 | 79 | 30 | 22 | 13 |
| 19 | 112 | 9 | 32 | 3 |
| 24 | 100 | 4.4 | 27 | 2.3 |
| 28 | 113 | 5.26 | 34 | 1.9 |

PHH from donor 8181 were infected with patient viruses P21 for three days before serially diluted Example 8 was added to the cells. After two weeks of the treatment the compound was removed and cells were followed for another 14 days. Activity of the compound was monitored during the course of the experiment using HBsAg and HBeAg readout. The medium with/without compound was replenished every 3-4 days. Day 0—compound was added to the infected cells.

TABLE 13
Effect of removal of 2 μM Example
7 on HBsAg and HBeAg levels

| | |
|---|---|
| HBsAg (ng/mL) | HBeAg (ng/mL) |

| Day | 0 nM | 2000 nM | 0 nM | 2000 nM |
|---|---|---|---|---|
| 0 | 0 | 0 | 0 | 0 |
| 5 | 26.6 | 20.6 | 16.0 | 9.2 |
| 10 | 359.1 | 149.2 | 74.3 | 25.1 |
| 14 | 315.2 | 68.6 | 74.2 | 13.7 |
| 19 | 332.0 | 26.8 | 72.5 | 7.0 |
| 24 | 189.2 | 11.7 | 53.2 | 5.3 |
| 28 | 178.9 | 19.1 | 39.0 | 5.4 |

PHH donor 8181 was infected with patient viruses P21 for three days before serially diluted Example 7 was added to the cells. After two weeks of the treatment the compound was removed and cells were followed for another 14 days. Activity of the compound was monitored during the course of the experiment using HBsAg and HBeAg readout. The medium with/without compound was replenished every 3-4 days. Day 0—compound was added to the infected cells.

1. A method of treating HBV comprising administering a KDM5 inhibitor to a patient infected with HBV.

2. The method of claim 1, wherein the KDM5 inhibitor is administered to the patient once daily.

3. The method of claim 1 wherein the KDM5 inhibitor is administered as a pulse dosing regimen.

4. The method of claim 1, wherein the KDM5 inhibitor inhibits at least 2 isoforms of KDM5, selected from the group consisting of KDM5a, KDM5b, KDM5c, and KDM5d.

5. The method of claim 1, wherein the KDM5 inhibitor inhibits at least 3 isoforms of KDM5, selected from the group consisting of KDM5a, KDM5b, KDM5c, and KDM5d.

6. The method of claim 1, wherein the KDM5 inhibitor inhibits 4 isoforms of KDM5, selected from the group consisting of KDM5a, KDM5b, KDM5c, and KDM5d.

7. The method of claim 1, further comprising administering an additional therapeutic agent to the patient.

8. The method of claim 7, wherein the additional therapeutic agent is administered separately from the KDM5 inhibitor.

9. The method of claim 7, wherein the additional therapeutic agent is administered in combination with the KDM5 inhibitor.

10. The method of claim 7 wherein the additional agent is selected from the group consisting of adefovir, tenofovir disoproxil, tenofovir disoproxil fumarate, tenofovir alafenamide hemifumarate, entecavir, interferon, lamivudine and telbivudine.

11. The method of claim 1, wherein the KDM5 inhibitor is a compound of Formula I^{a}: 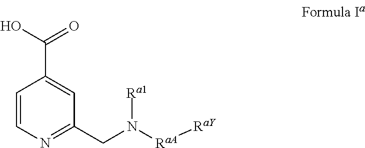
or a pharmaceutically acceptable salt thereof, wherein:
R^{aA} is —CHR^{a2}C(O)—, C₁₋₈ alkylene, C₂₋₈ alkenylene, C₂₋₈ alkynylene, C₃₋₁₀ cycloalkylene, heterocyclylene, heteroarylene or arylene;
wherein each alkylene, alkenylene, alkynylene, cycloalkylene, heterocyclylene, heteroarylene and arylene may optionally be substituted with one or more R^{a3};
R^{aY} is —H, —NR^{a6}R^{a7}, —OR^{a7}, C₁₋₈ alkyl, C₂₋₈ alkenyl, C₂₋₈ alkynyl, C₃₋₁₀ cycloalkyl, heterocyclyl, heteroaryl or aryl;
wherein each alkyl, alkenyl, alkynyl, cycloalkyl, heterocyclyl, heteroaryl and aryl may optionally be substituted with one or more R^{a3} and may form a cyclic structure with R^{a2};
R^{a1} is —H, C₁₋₈ alkyl, C₂₋₈ alkenyl, C₂₋₈ alkynyl, or C₃₋₁₀ cycloalkyl;
wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; or
wherein each alkyl, alkenyl, alkynyl and cycloalkyl may be optionally substituted with one or more —H or C₁₋₄ alkyl; or
wherein R^{a1} with —R^{aA}—R^{aY} forms a nitrogen containing optionally substituted heterocyclic group wherein the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl;
R^{a2} is —H, C₁₋₈ alkyl, C₂₋₈ alkenyl, C₂₋₈ alkynyl or C₃₋₁₀ cycloalkyl;
wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl, and may form a cyclic structure with R^{aY};
each R^{a3} is independently C₁₋₆ alkyl, C₁₋₄ fluoroalkyl, C₁₋₄ hydroxyalkyl, C₂₋₆ alkenyl, C₂₋₆ alkynyl, C₃₋₁₀
cycloalkyl, —R^{aZ}-heterocyclyl, —R^{aZ}-aryl, —R^{aZ}-heteroaryl, —R^{aZ}—NR^{a6}R^{a7}, —R^{aZ}—C(═O)—NR^{a6}R^{a7}, —R^{aZ}—NR^{a6}—C(═O)—R^{a7}, —R^{aZ}—C(═O)—R^{a7}, —R—OR^{a7},
halogen, —R^{aZ}—SR^{a7}, —R^{aZ}—SOR^{a7}, —R^{aZ}—SO₂R^{a7}, —R^{aZ}—SO₂NR^{a6}R^{a7} or —R^{aZ}—COOR^{a7};
wherein any heterocyclyl may be substituted with one or more R^{a4}; and
wherein any heteroaryl and any aryl may be substituted with one or more R^{a5};
R^{aZ} is a single bond, C₁₋₄ alkylene, heterocyclylene or C₃₋₆ cycloalkylene;
each R^{a4} is independently C₁₋₆ alkyl, C₁₋₄ fluoroalkyl, C₁₋₄ hydroxyalkyl, C₁₋₄ alkoxy, C₃₋₁₀ cycloalkyl, —N(R^{a1})₂, carbamoyl or —OH;
each R^{a5} is independently C₁₋₆ alkyl, C₁₋₄ fluoroalkyl, C₁₋₄ hydroxyalkyl, C₁₋₄ alkoxy, C₃₋₆ cycloalkyl, —CN, —F, —Cl, —Br, carbamoyl or —OH;
each of R^{a6} and R^{a7} is independently —H, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl,
C₃₋₁₀ cycloalkyl, —R^{aZ}-heterocyclyl, —R^{aZ}-heteroaryl or —R^{aZ}-aryl;
wherein each alkyl, alkenyl, alkynyl, cycloalkyl, heterocyclyl, heteroaryl and aryl may optionally be substituted with one or more independently selected R^{a8}; or
wherein R^{a6} and R^{a7} may together with the N-atom to which they are attached form an N-heterocyclic ring optionally substituted with one or more independently selected R^{a8};
each R^{a8} is independently C₁₋₆ alkyl, C₁₋₄ fluoroalkyl, C₁₋₄ hydroxyalkyl, C₂₋₆ alkenyl, C₂₋₆ alkynyl, C₃₋₁₀
cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9},
halogen, —CN, —R^{aZ}—SR^{a9}, —R^{aZ}—SOR^{a9}, —R^{aZ}—SO₂R^{a9} or —R^{aZ}—COOR^{a9};
wherein each alkyl, alkenyl, alkynyl, cycloalkyl, heterocyclics, heteroaryl and aryl may optionally be substituted with one or more C₁₋₄ alkyl, C₁₋₄ fluoroalkyl, C₁₋₄ hydroxyalkyl, C₃₋₆
cycloalkyl, —R^{aZ}-heterocyclyl, —R^{aZ}-heteroaryl, —R^{aZ}-aryl, —R^{aZ}—NR^{a10}R^{a11}, —R^{a}C(═O)—NR^{a10}R^{a11}, —R^{aZ}—OR^{a9},
halogen, —CN, —R^{aZ}—SR^{a9}, —R^{aZ}—SOR^{a9}, —R^{aZ}—SO₂R^{a9} or —R^{aZ}—COOR^{a9};
wherein any heterocyclyl may be further substituted with one or more R^{a4} as defined above, and
wherein any heteroaryl and any aryl may be further substituted with one or more R^{a5} as defined above;
each R^{a9} is independently —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-aryl or —R^{aZ}-heteroaryl;
wherein any heterocyclyl may be substituted with one or more R^{a4} as defined above; and
wherein any heteroaryl and any aryl may be substituted with one or more R^{a5} as defined above; and
each of R^{a10} and R^{a11} is independently —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl or aryl;
wherein any heterocyclyl may be substituted with one or more R^{a4} as defined above; and
wherein any heteroaryl and any aryl may be substituted with one or more R^{a5} as defined above; or
wherein R^{a10} and R^{a11} may together with the N-atom to which they are attached form an N-heterocyclic ring optionally substituted with one or more R^{a4} as defined above.

12. The method of claim 1, wherein the KDM5 inhibitor is a compound of Formula I^{a1}: 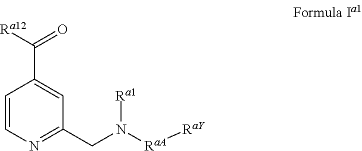
wherein:
R^{a12} is of the form (R^{a13})₂N- or of the form R^{a13}O—, wherein each R^{a13} independently may be selected from C₁₋₈ alkyl, C₂₋₈ alkenyl, C₂₋₈ alkynyl, C₃₋₁₀ cycloalkyl, and aryloxy wherein each alkyl, alkenyl, alkynyl, cycloalkyl and aryloxy may be optionally substituted with one or more selected from —OH, aryl, C₁₋₆ alkoxy, heteroaryl, aryloxy, heteroaryloxy, F, a sulphonamide moiety, and C₃₋₆ cycloalkyl; and one R^{a13} in (R^{a13})₂N-may be —H;
R^{aA} is —CHR^{a2}C(O)—, C₁₋₈ alkylene, C₂₋₈ alkenylene, C₂₋₈ alkynylene, C₃₋₁₀ cycloalkylene, heterocyclylene, heteroarylene or arylene;
wherein each alkylene, alkenylene, alkynylene, cycloalkylene, heterocyclylene, heteroarylene and arylene may optionally be substituted with one or more R^{a3};
R^{aY} is —H, —NR^{a6}R^{a7}, —OR^{a7}, C₁₋₈ alkyl, C₂₋₈ alkenyl, C₂₋₈ alkynyl, C₃₋₁₀ cycloalkyl, heterocyclyl, heteroaryl or aryl;
wherein each alkyl, alkenyl, alkynyl, cycloalkyl, heterocyclyl, heteroaryl and aryl may optionally be substituted with one or more R^{a3} and may form a cyclic structure with R^{a2};
R^{a1} is —H, C₁₋₈ alkyl, C₂₋₈ alkenyl, C₂₋₈ alkynyl, or C₃₋₁₀ cycloalkyl;
wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl; or
wherein each alkyl, alkenyl, alkynyl and cycloalkyl may be optionally substituted with one or more —H or C₁₋₄ alkyl; or
wherein R^{a1} with —R^{aA}—R^{aY} forms a nitrogen containing optionally substituted heterocyclic group wherein the optional substitution may be C₁₋₈ alkyl, C₂₋₈ alkenyl, C₂₋₈ alkynyl, or C₃₋₁₀ cycloalkyl, which alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl;
R^{a2} is —H, C₁₋₈ alkyl, C₂₋₈ alkenyl, C₂₋₈ alkynyl or C₃₋₁₀ cycloalkyl;
wherein each alkyl, alkenyl, alkynyl and cycloalkyl may be optionally substituted with one or more —OH, aryl, C₁₋₆ alkoxy, heteroaryl, aryloxy, heteroaryloxy, F or C₃₋₆ cycloalkyl, and may form a cyclic structure with R^{aY};
each R^{a3} is independently C₁₋₆ alkyl, C₁₋₄ fluoroalkyl, C₁₋₄ hydroxyalkyl, C₂₋₆ alkenyl, C₂₋₆ alkynyl, C₃₋₁₀
cycloalkyl, —R^{aZ}-heterocyclyl, —R^{aZ}-aryl, —R^{aZ}-heteroaryl, —R^{aZ}—NR^{a6}R^{a7}, —R^{aZ}—C(═O)—NR^{a6}R^{a7}, —R^{aZ}—NR^{a6}—C(═O)—R^{a7}, —R^{aZ}—C(═O)—R^{a7}, —R^{aZ}—OR^{a7}
halogen, —R^{aZ}—SR^{a7}, —R^{aZ}—SOR^{a7}, —R^{aZ}—SO₂R^{a7}, —R^{aZ}—SO₂NR^{a6}R^{a7} or —R^{aZ}—COOR^{a7};
wherein any heterocyclyl may be substituted with one or more R^{a4}; and
wherein any heteroaryl and any aryl may be substituted with one or more R^{a5};
R^{aZ} is a single bond, C₁₋₄ alkylene, heterocyclylene or C₃₋₆ cycloalkylene;
each R^{a4} is independently C₁₋₆ alkyl, C₁₋₄ fluoroalkyl, C₁₋₄ hydroxyalkyl, C₁₋₄ alkoxy, C₃₋₁₀ cycloalkyl, —N(R^{a1})₂, carbamoyl or —OH;
each R^{a5} is independently C₁₋₆ alkyl, C₁₋₄ fluoroalkyl, C₁₋₄ hydroxyalkyl, C₁₋₄ alkoxy, C₃₋₆ cycloalkyl, —CN, —F, —Cl, —Br, carbamoyl or —OH;
each of R^{a6} and R^{a7} is independently —H, C₁₋₈ alkyl, C₁₋₄ fluoroalkyl, C₁₋₄ perfluoroalkyl, C₁₋₄ hydroxyalkyl, C₂₋₈ alkenyl, C₂₋₈ alkynyl,
C₃₋₁₀ cycloalkyl, —R^{aZ}-heterocyclyl, —R^{aZ}-heteroaryl or —R^{aZ}-aryl;
wherein each alkyl, alkenyl, alkynyl, cycloalkyl, heterocyclyl, heteroaryl and aryl may optionally be substituted with one or more independently selected R^{a8}; or
wherein R^{a6} and R^{a7} may together with the N-atom to which they are attached form an N-heterocyclic ring optionally substituted with one or more independently selected R^{a8};
each R^{a8} is independently C₁₋₆ alkyl, C₁₋₄ fluoroalkyl, C₁₋₄ hydroxyalkyl, C₂₋₆ alkenyl, C₂₋₆ alkynyl, C₃₋₁₀
cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9},
halogen, —CN, —R^{aZ}—SR^{a9}, —R^{aZ}—SOR^{a9}, —R^{aZ}—SO₂R^{a9} or —R^{aZ}—COOR^{a9};
wherein each alkyl, alkenyl, alkynyl, cycloalkyl, heterocyclics, heteroaryl and aryl may optionally be substituted with one or more C₁₋₄ alkyl, C₁₋₄ fluoroalkyl, C₁₋₄ hydroxyalkyl, C₃₋₆
cycloalkyl, —R^{aZ}-heterocyclyl, —R^{aZ}-heteroaryl, —R^{aZ}-aryl, —R^{aZ}—NR^{a10}R^{a11}, —R^{aZ}—C(═O)—NR^{a10}R^{a11}, —R^{aZ}—OR^{a9},
halogen, —CN, —R^{aZ}—SR^{a9}, —R^{aZ}—SOR^{a9}, —R^{aZ}—SO₂R^{a9} or —R^{aZ}—COOR^{a9};
wherein any heterocyclyl may be further substituted with one or more R^{a4} as defined above, and
wherein any heteroaryl and any aryl may be further substituted with one or more R^{a5} as defined above;
each R^{a9} is independently —H, C₁₋₈ alkyl, C₁₋₄ fluoroalkyl, C₁₋₄ hydroxyalkyl, C₂₋₈ alkenyl, C₂₋₈ alkynyl, C₃₋₁₀ cycloalkyl, —R^{aZ}-heterocyclyl, —R^{aZ}-aryl or —R^{aZ}-heteroaryl;
wherein any heterocyclyl may be substituted with one or more R^{a4} as defined above; and
wherein any heteroaryl and any aryl may be substituted with one or more R^{a5} as defined above; and
each of R^{a10} and R^{a11} is independently —H, C₁₋₆ alkyl, C₁₋₄ fluoroalkyl, C₁₋₄ hydroxyalkyl, C₂₋₈ alkenyl, C₂₋₈ alkynyl, C₃₋₁₀ cycloalkyl, heterocyclyl, heteroaryl or aryl;
wherein any heterocyclyl may be substituted with one or more R^{a4} as defined above; and
wherein any heteroaryl and any aryl may be substituted with one or more R^{a5} as defined above; or
wherein R^{a10} and R^{a11} may together with the N-atom to which they are attached form an N-heterocyclic ring optionally substituted with one or more R^{a4} as defined above;
or a pharmaceutically acceptable salt thereof.

13. The method of claim 1, wherein the KDM5 inhibitor is 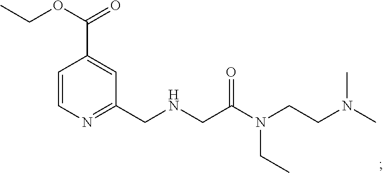 [SMILES: CCOC(=O)c1ccnc(CNCC(=O)N(CC)CCN(C)C)c1]
or a pharmaceutically acceptable salt thereof.

14. The method of claim 1, wherein the KDM5 inhibitor is 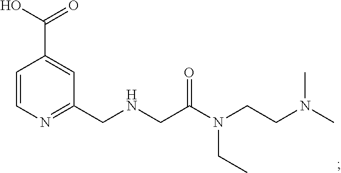 [SMILES: CCN(CCN(C)C)C(=O)CNCc1cc(C(=O)O)ccn1]
or a pharmaceutically acceptable salt thereof.

15-37. (canceled)
